SEQUENCE LISTING (52 sequences)

SEQ ID NO: 1 - DNA, 1140 bp, Mus musculus
agcaggtttgttacagaaaccttagttaaggtttgttgagggttttttttctctctctctctcttaattggctgtcccaatccatccttctataaatagaaaagagagacagggagtgtgtgtggtttcattactaaggtaaagacacttgagctacacacacttgatccctgaacatgaaatctaagaggttgaacgatcacagtttcaggactatataaggtggtgaaagaccatctgcttcgtttttctgtttgttcctacaactctttccctccgcttgattttaactctaaattggtgagtagctggtgggctcaccagactccgagatcctcttctctgcacgcactgtattagacttggcacccgggaggattttcacctctgctgcatgggctaatcttccacaagggatctgtggtattgcaatctcgggttgatgcatgacggtgatgttgtgtttatagcatggctaaggtttagctgcctatgatgattggttagggaaggataatttttgctagaagattggactttagggaaaaaaaaccccacttttatttgcttttagaattttaaaagactgggccatgtagctcaggctggtttggagttcattatgtagtcaaggatgctcttggactctttagcatcctcctcctcctctttttcctcctcctccttcttgttcttcttcttgttcttcctcttccccttctcttcccccttctctccctcttcctcctcttcctcctccttcttgttcttcttcctcttccccttctcctctcccccttgtcctcctcttccttctcctcctcctcctcttcttctttctgagtaccaagattgcaagtgtgcacacgatgaccagcttggtctttctttgtcttttttttttaacttcaattttggagtgaattcaagagcaaccatgtagtcaagaggtggctggagtcttttctgtatctgggtttggtttagtactctgccccatcacttaacaggtccttatggccacatcttaaaaaaattctagagatacacggtgtcggtgagtggctgagaatgtgtggtcttcccatttctctgtcaccgtggctcacatcttgtttcctctgttcggccaggtagaaa

SEQ ID NO: 2 - DNA, 2500 bp, Homo sapiens
ttttaccacaataattaaaaagaacagtctagcacagtgctggccatataaaggctcaataaatgtttgctgaaagttaaaaaaaaaaaaaaaaaaaaaaaagccaggcgcagtggttcattcctgtaatcccagcactttaggaggatgaggtgggagaattacttgagcccaggagttcgagaccagcctaggcaacatggcaaaaccctgtcaaaaccctgtctctccaaaaaatatgcatatttaaaaaattagccaggcatggtggtgtgtgcctgtagtaccagctactcgggagactgaagtgggaggatcgcttgagcctgggaggtcaaggctgcaatgagctgagatcgtgccactgcactccagccggggcaacagagcaagaccctgtcacaacagaaacaaaatcttgaggtgtctagtcctggcctcagcctcagaatatttgtttctgaacatgttagttttgggggttggggatgctggtttgatttcctcctttttgccttttgagtgtgtgcaatttatggtatagctgggaaacgtcaaagtcaagagttttgtaggaaagtcacgtcacttagccctgtctcctgtgccgggtgagacctgtgtgtgcacttggtgacaatggctttgagtctgtcaactccagactgaggtcagccttacacacccatagttcccaaagctgaaaacaggcctgcctccaacggtacctgctaatatcaggggagccttttcagcttacagagcaccctgtatgtgtttgtcttagttcaggccaccatctccaccttaccaggcatctagaaccttctccacactttgccaacagggttcgtttgcagaattgaaatcttagttaaggtttgttgaagtttgttgttgttttttttttttttttacaattggctgttcccacccacattcccttgagacataaatagaaaaaaaaaaaaaaagaggtttcatgagtaagacaagacatttgagctgcatccacttgatccttgaaaaggaaatctaagaggttgtaactatcactttttctagcctatataaggtaggtcagtaaggtagcaaaaacacatctgttgttttgctccttcaactctttttcctgattcttcctggggggaaaccgaaaacggtgagtaactggtggacacatcagaccccagactcttttcttcactgcatgcattcatattaggctcaggtgcttagactcctgttttccggtggctctgacacctggaaggattttaatctctgggagatgggcttttcatccatctgcttcccacctttcaggacaggtgcatgccttcttccacagaatgtctgcaagcagcccaaactgtatcctttcccacgtggaatttgcaacattgcatctctcgggctgctgtaggaaaatgccagtgcatgtgtaacatggtttacggctgcctatgcaaatgactgattatgtcagtataatttttataagaaaacaattgaatccttctttgggtcattttttttttccatttttggcatgtattcaaaagaaggctctgagacaaaaaaggctggggtgttttccgtatctggttttaatttggatattctgtcccgtcacttaatacaaaaccatgcttatcacattttaaaaattctagacaggcctggctcggtggcttgcatctgtcatcccagcactttgtgaggccaaggcaggcagatcacctgaggtcaggagctcaagaccagcctggccaacatggcaaaaccccgtctctactaaaaacacaaaaattagccaggcatggtagtgcgcacctgtaatcccagctactgggaaggcttaggcaggagaatcacttgagcccaggaggcggaggttgcggtgagccgagatcacgctcttgcactccagcctgggtgacagagtgagactccgtcttaatttaaaaaaaaaaataatctagacacacatacagtttcagtgggcctgggaagatgtgtttcccctggatgtgcacattcctgtttgtggcttatcgcctctcatttattctgtgtgagtaggtagaaaatgagcatcacggacgtgctcagtgctgacgacattgcagcagcgctccaggaatgccgaggtagaggggacgtgaggcgggggtgggatttcctcacagctttgcacctccagcgagtcaacacaaaatcaaaatgtaggccaggcggccagacgcagtggctcacacctgtaatcccagcactttgggaggccgaggcgggtggatcacgaggtcaggagttcgagaccagcctggccaagatggtgaaaccccatctctactaaaaatacaaaaaaattaaccgggcgtggtggtgggtgcctgtaatcccagctactcgggaggctgaggcagagaattgcttgaacccgggaggcagaagttgcagtgagctgagatcatgccactgcactccagcctgggca

SEQ ID NO: 3 - DNA, 833 bp, Artificial Sequence
  Synthetic Construct
gttcccaaagctgaaaacaggcctgcctccaacggtacctgctaatatcaggggagccttttcagcttacagagcaccctgtatgtgtttgtcttagttcaggcaccttaccaggcatctagaaccttctccacactttgccaacagggttcgtttgcagaattgaaatcttagttaaggtttgttgaagtttgttgttgttttttttttttttttacaattggctgttcccacccacattcccttgagacataaatagaaaaaaaaaaaaaaagaggtttcatgagtaagacaagacatttgagctgcatccacttgatccttgaaaaggaaatctaagaggttgtaactatcactttttctagcctatataaggtaggtcagtaaggtagcaaaaacacatctgttgttttgctccttcaactctttttcctgattcttcctggggggaaaccgaaaacggtgagtaactggtggacacatcagaccccagactcttttcttcactgcatgcattcatattaggctcaggtgcttagactcctgttttccggtttacggctgcctatgcaaatgactgattatgtcagtataatttttataagaaaacaattgaatccttctttgggtcattttttttttccatttttggcatgtatgtgcacattcctgtttgtggcttatcgcctctcatttattctgtgtgagtaggtagaaaatgagcatcacggacgtgctcagtgctgacgacattgcagcagcgctccaggaatgccgaggtagaggggacgtgaggcgggggtgggatttcctcacagctttgcacctccagc

SEQ ID NO: 4 - protein, 1775 aa, Homo sapiens
MALSLWPLLLLLLLLLLLSFAVTLAPTGPHSLDPGLSFLKSLLSTLDQAPQGSLSRSRFFTFLANISSSFEPGRMGEGPVGEPPPLQPPALRLHDFLVTLRGSPDWEPMLGLLGDMLALLGQEQTPRDFLVHQAGVLGGLVEVLLGALVPGGPPTPTRPPCTRDGPSDCVLAADWLPSLLLLLEGTRWQALVQVQPSVDPTNATGLDGREAAPHFLQGLLGLLTPTGELGSKEALWGGLLRTVGAPLYAAFQEGLLRVTHSLQDEVFSILGQPEPDTNGQCQGGNLQQLLLWGVRHNLSWDVQALGFLSGSPPPPPALLHCLSTGVPLPRASQPSAHISPRQRRAITVEALCENHLGPAPPYSISNFSIHLLCQHTKPATPQPHPSTTAICQTAVWYAVSWAPGAQGWLQACHDQFPDEFLDAICSNLSFSALSGSNRRLVKRLCAGLLPPPTSCPEGLPPVPLTPDIFWGCFLENETLWAERLCGEASLQAVPPSNQAWVQHVCQGPTPDVTASPPCHIGPCGERCPDGGSFLVMVCANDTMYEVLVPFWPWLAGQCRISRGGNDTCFLEGLLGPLLPSLPPLGPSPLCLTPGPFLLGMLSQLPRCQSSVPALAHPTRLHYLLRLLTFLLGPGAGGAEAQGMLGRALLLSSLPDNCSFWDAFRPEGRRSVLRTIGEYLEQDEEQPTPSGFEPTVNPSSGISKMELLACFSPVLWDLLQREKSVWALQILVQAYLHMPPENLQQLVLSAEREAAQGFLTLMLQGKLQGKLQVPPSEEQALGRLTALLLQRYPRLTSQLFIDLSPLIPFLAVSDLMRFPPSLLANDSVLAAIRDYSPGMRPEQKEALAKRLLAPELFGEVPAWPQELLWAVLPLLPHLPLENFLQLSPHQIQALEDSWPAAGLGPGHARHVLRSLVNQSVQDGEEQVRRLGPLACFLSPEELQSLVPLSDPTGPVERGLLECAANGTLSPEGRVAYELLGVLRSSGGAVLSPRELRVWAPLFSQLGLRFLQELSEPQLRAMLPVLQGTSVTPAQAVLLLGRLLPRHDLSLEELCSLHLLLPGLSPQTLQAIPRRVLVGACSCLAPELSRLSACQTAALLQTFRVKDGVKNMGTTGAGPAVCIPGQPIPTTWPDCLLPLLPLKLLQLDSLALLANRRRYWELPWSEQQAQFLWKKMQVPTNLTLRNLQALGTLAGGMSCEFLQQINSMVDFLEVVHMIYQLPTRVRGSLRACIWAELQRRMAMPEPEWTTVGPELNGLDSKLLLDLPIQLMDRLSNESIMLVVELVQRAPEQLLALTPLHQAALAERALQNLAPKETPVSGEVLETLGPLVGFLGTESTRQIPLQILLSHLSQLQGFCLGETFATELGWLLLQESVLGKPELWSQDEVEQAGRLVFTLSTEAISLIPREALGPETLERLLEKQQSWEQSRVGQLCREPQLAAKKAALVAGVVRPAAEDLPEPVPNCADVRGTFPAAWSATQIAEMELSDFEDCLTLFAGDPGLGPEELRAAMGKAKQLWGPPRGFRPEQILQLGRLLIGLGDRELQELILVDWGVLSTLGQIDGWSTTQLRIVVSSFLRQSGRHVSHLDFVHLTALGYTLCGLRPEELQHISSWEFSQAALFLGTLHLQCSEEQLEVLAHLLVLPGGFGPISNWGPEIFTEIGTIAAGIPDLALSALLRGQIQGVTPLAISVIPPPKFAVVFSPIQLSSLTSAQAVAVTPEQMAFLSPEQRRAVAWAQHEGKESPEQQGRSTAWGLQDWSRPSWSLVLTISFLGHLL

SEQ ID NO: 5 - protein, 1809 aa, Mus musculus
MALSLQPQLLLLLSLLPQEVTSAPTGPQSLDAGLSLLKSFVATLDQAPQRSLSQSRFSAFLANISSSFQLGRMGEGPVGEPPPLQPPALRLHDFLVTLRGSPDWEPMLGLLGDVLALLGQEQTPRDFLVHQAGVLGGLVEALLGALVPGGPPAPTRPPCTRDGPSDCVLAADWLPSLMLLLEGTRWQALVQLQPSVDPTNATGLDGREPAPHFLQGLLGLLTPAGELGSEEALWGGLLRTVGAPLYAAFQEGLLRVTHSLQDEVFSIMGQPEPDASGQCQGGNLQQLLLWGMRNNLSWDARALGFLSGSPPPPPALLHCLSRGVPLPRASQPAAHISPRQRRAISVEALCENHSGPEPPYSISNFSIYLLCQHIKPATPRPPPTTPRPPPTTPQPPPTTTQPIPDTTQPPPVTPRPPPTTPQPPPSTAVICQTAVWYAVSWAPGARGWLQACHDQFPDQFLDMICGNLSFSALSGPSRPLVKQLCAGLLPPPTSCPPGLIPVPLTPEIFWGCFLENETLWAERLCVEDSLQAVPPRNQAWVQHVCRGPTLDATDFPPCRVGPCGERCPDGGSFLLMVCANDTLYEALVPFWAWLAGQCRISRGGNDTCFLEGMLGPLLPSLPPLGPSPLCLAPGPFLLGMLSQLPRCQSSVPALAHPTRLHYLLRLLTFLLGPGTGGAETQGMLGQALLLSSLPDNCSFWDAFRPEGRRSVLRTVGEYLQREEPTPPGLDSSLSLGSGMSKMELLSCFSPVLWDLLQREKSVWALRTLVKAYLRMPPEDLQQLVLSAEMEAAQGFLTLMLRSWAKLKVQPSEEQAMGRLTALLLQRYPRLTSQLFIDMSPLIPFLAVPDLMRFPPSLLANDSVLAAIRDHSSGMKPEQKEALAKRLLAPELFGEVPDWPQELLWAALPLLPHLPLESFLQLSPHQIQALEDSWPVADLGPGHARHVLRSLVNQSMEDGEEQVLRLGSLACFLSPEELQSLVPLSDPMGPVEQGLLECAANGTLSPEGRVAYELLGVLRSSGGTVLSPRELRVWAPLFPQLGLRFLQELSETQLRAMLPALQGASVTPAQAVLLFGRLLPKHDLSLEELCSLHPLLPGLSPQTLQAIPKRVLVGACSCLGPELSRLSACQIAALLQTFRVKDGVKNMGAAGAGSAVCIPGQPTTWPDCLLPLLPLKLLQLDAAALLANRRLYRQLPWSEQQAQFLWKKMQVPTNLSLRNLQALGNLAGGMTCEFLQQISSMVDFLDVVHMLYQLPTGVRESLRACIWTELQRRMTMPEPELTTLGPELSELDTKLLLDLPIQLMDRLSNDSIMLVVEMVQGAPEQLLALTPLHQTALAERALKNLAPKETPISKEVLETLGPLVGFLGIESTRRIPLPILLSHLSQLQGFCLGETFATELGWLLLQEPVLGKPELWSQDEIEQAGRLVFTLSAEAISSIPREALGPETLERLLGKHQSWEQSRVGHLCGESQLAHKKAALVAGIVHPAAEGLQEPVPNCADIRGTFPAAWSATQISEMELSDFEDCLSLFAGDPGLGPEELRAAMGKAKQLWGPPRGFRPEQILQLGRLLIGLGERELQELTLVDWGVLSSLGQIDGWSSMQLRAVVSSFLRQSGRHVSHLDFIYLTALGYTVCGLRPEELQHISSWEFSQAALFLGSLHLPCSEEQLEVLAYLLVLPGGFGPVSNWGPEIFTEIGTIAAGIPDLALSALLRGQIQGLTPLAISVIPAPKFAVVFNPIQLSSLTRGQAVAVTPEQLAYLSPEQRRAVAWAQHEGKEIPEQLGRNSAWGLYDWFQASWALALPVSIFGHLL

SEQ ID NO: 6 - DNA, 5328 bp, Homo sapiens
atggctctcagcctctggcccctgctgctgctgctgctgctgctgctgctgctgtcctttgcagtgactctggcccctactgggcctcattccctggaccctggtctctccttcctgaagtcattgctctccactctggaccaggctccccagggctccctgagccgctcacggttctttacattcctggccaacatttcttcttcctttgagcctgggagaatgggggaaggaccagtaggagagcccccacctctccagccgcctgctctgcggctccatgattttctagtgacactgagaggtagccccgactgggagccaatgctagggctgctaggggatatgctggcactgctgggacaggagcagactccccgagatttcctggtgcaccaggcaggggtgctgggtggacttgtggaggtgctgctgggagccttagttcctgggggcccccctaccccaactcggcccccatgcacccgtgatgggccgtctgactgtgtcctggctgctgactggttgccttctctgctgctgttgttagagggcacacgctggcaagctctggtgcaggtgcagcccagtgtggaccccaccaatgccacaggcctcgatgggagggaggcagctcctcactttttgcagggtctgttgggtttgcttaccccaacaggggagctaggctccaaggaggctctttggggcggtctgctacgcacagtgggggcccccctctatgctgcctttcaggaggggctgctccgtgtcactcactccctgcaggatgaggtcttctccattttggggcagccagagcctgataccaatgggcagtgccagggaggtaaccttcaacagctgctcttatggggcgtccggcacaacctttcctgggatgtccaggcgctgggctttctgtctggatcaccacccccaccccctgccctccttcactgcctgagcacgggcgtgcctctgcccagagcttctcagccgtcagcccacatcagcccacgccaacggcgagccatcactgtggaggccctctgtgagaaccacttaggcccagcaccaccctacagcatttccaacttctccatccacttgctctgccagcacaccaagcctgccactccacagccccatcccagcaccactgccatctgccagacagctgtgtggtatgcagtgtcctgggcaccaggtgcccaaggctggctacaggcctgccacgaccagtttcctgatgagtttttggatgcgatctgcagtaacctctccttttcagccctgtctggctccaaccgccgcctggtgaagcggctctgtgctggcctgctcccaccccctaccagctgccctgaaggcctgccccctgttcccctcaccccagacatcttttggggctgcttcttggagaatgagactctgtgggctgagcgactgtgtggggaggcaagtctacaggctgtgccccccagcaaccaggcttgggtccagcatgtgtgccagggccccaccccagatgtcactgcctccccaccatgccacattggaccctgtggggaacgctgcccggatgggggcagcttcctggtgatggtctgtgccaatgacaccatgtatgaggtcctggtgcccttctggccttggctagcaggccaatgcaggataagtcgtgggggcaatgacacttgcttcctagaagggctgctgggcccccttctgccctctctgccaccactgggaccatccccactctgtctgacccctggccccttcctccttggcatgctatcccagttgccacgctgtcagtcctctgtcccagctcttgctcaccccacacgcctacactatctcctccgcctgctgaccttcctcttgggtccaggggctgggggcgctgaggcccaggggatgctgggtcgggccctactgctctccagtctcccagacaactgctccttctgggatgcctttcgcccagagggccggcgcagtgtgctacggacgattggggaatacctggaacaagatgaggagcagccaaccccatcaggctttgaacccactgtcaaccccagctctggtataagcaagatggagctgctggcctgctttagtcctgtgctgtgggatctgctccagagggaaaagagtgtttgggccctgcagattctagtgcaggcgtacctgcatatgcccccagaaaacctccagcagctggtgctttcagcagagagggaggctgcacagggcttcctgacactcatgctgcaggggaagctgcaggggaagctgcaggtaccaccatccgaggagcaggccctgggtcgcctgacagccctgctgctccagcggtacccacgcctcacctcccagctcttcattgacctgtcaccactcatccctttcttggctgtctctgacctgatgcgcttcccaccatccctgttagccaacgacagtgtcctggctgccatccgggattacagcccaggaatgaggcctgaacagaaggaggctctggcaaagcgactgctggcccctgaactgtttggggaagtgcctgcctggccccaggagctgctgtgggcagtgctgcccctgctcccccacctccctctggagaactttttgcagctcagccctcaccagatccaggccctggaggatagctggccagcagcaggtctggggccagggcatgcccgccatgtgctgcgcagcctggtaaaccagagtgtccaggatggtgaggagcaggtacgcaggcttgggcccctcgcctgtttcctgagccctgaggagctgcagagcctagtgcccctgagtgatccaacggggccagtagaacgggggctgctggaatgtgcagccaatgggaccctcagcccagaaggacgggtggcatatgaacttctgggtgtgttgcgctcatctggaggagcggtgctgagcccccgggagctgcgggtctgggcccctctcttctctcagctgggcctccgcttccttcaggagctgtcagagccccagcttagagccatgcttcctgtcctgcagggaactagtgttacacctgctcaggctgtcctgctgcttggacggctccttcctaggcacgatctatccctggaggaactctgctccttgcaccttctgctaccaggcctcagcccccagacactccaggccatccctaggcgagtcctggtcggggcttgttcctgcctggcccctgaactgtcacgcctctcagcctgccagaccgcagcactgctgcagacctttcgggttaaagatggtgttaaaaatatgggtacaacaggtgctggtccagctgtgtgtatccctggtcagcctattcccaccacctggccagactgcctgcttcccctgctcccattaaagctgctacaactggattccttggctcttctggcaaatcgaagacgctactgggagctgccctggtctgagcagcaggcacagtttctctggaagaagatgcaagtacccaccaaccttaccctcaggaatctgcaggctctgggcaccctggcaggaggcatgtcctgtgagtttctgcagcagatcaactccatggtagacttccttgaagtggtgcacatgatctatcagctgcccactagagttcgagggagcctgagggcctgtatctgggcagagctacagcggaggatggcaatgccagaaccagaatggacaactgtagggccagaactgaacgggctggatagcaagctactcctggacttaccgatccagttgatggacagactatccaatgaatccattatgttggtggtggagctggtgcaaagagctccagagcagctgctggcactgacccccctccaccaggcagccctggcagagagggcactacaaaacctggctccaaaggagactccagtctcaggggaagtgctggagaccttaggccctttggttggattcctggggacagagagcacacgacagatccccctacagatcctgctgtcccatctcagtcagctgcaaggcttctgcctaggagagacatttgccacagagctgggatggctgctattgcaggagtctgttcttgggaaaccagagttgtggagccaggatgaagtagagcaagctggacgcctagtattcactctgtctactgaggcaatttccttgatccccagggaggccttgggtccagagaccctggagcggcttctagaaaagcagcagagctgggagcagagcagagttggacagctgtgtagggagccacagcttgctgccaagaaagcagccctggtagcaggggtggtgcgaccagctgctgaggatcttccagaacctgtgccaaattgtgcagatgtacgagggacattcccagcagcctggtctgcaacccagattgcagagatggagctctcagactttgaggactgcctgacattatttgcaggagacccaggacttgggcctgaggaactgcgggcagccatgggcaaagcaaaacagttgtggggtcccccccggggatttcgtcctgagcagatcctgcagcttggtaggctcttaataggtctaggagatcgggaactacaggagctgatcctagtggactggggagtgctgagcaccctggggcagatagatggctggagcaccactcagctccgcattgtggtctccagtttcctacggcagagtggtcggcatgtgagccacctggacttcgttcatctgacagcgctgggttatactctctgtggactgcggccagaggagctccagcacatcagcagttgggagttcagccaagcagctctcttcctcggcaccctgcatctccagtgctctgaggaacaactggaggttctggcccacctacttgtactgcctggtgggtttggcccaatcagtaactgggggcctgagatcttcactgaaattggcaccatagcagctgggatcccagacctggctctttcagcactgctgcggggacagatccagggcgttactcctcttgccatttctgtcatccctcctcctaaatttgctgtggtgtttagtcccatccaactatctagtctcaccagtgctcaggctgtggctgtcactcctgagcaaatggcctttctgagtcctgagcagcgacgagcagttgcatgggcccaacatgagggaaaggagagcccagaacagcaaggtcgaagtacagcctggggcctccaggactggtcacgaccttcctggtccctggtattgactatcagcttccttggccacctgctatga

SEQ ID NO: 7 - DNA, 5430 bp, Mus musculus
atggctctgagcctccagccccagctgctccttctcctgtcgctcctgccgcaggaagtgacttcagcccctactgggcctcagtctttggatgctggtctctcccttctgaagtcattcgtagccactctggaccaagctcctcagcgttccctcagccagtcacggttctctgcgttcctggccaacatttcttcatccttccagcttgggaggatgggggagggaccggtgggagagcccccacctctccagccccctgcacttcgacttcatgatttcctcgtgacactgagaggtagcccagactgggagccaatgctagggcttctgggagatgtgctggcactcctgggacaggaacagactccccgggactttttggtgcaccaggcaggtgtactgggtggacttgtagaggcattgttgggagcgttagttcctggaggcccccctgcccccactcgacccccatgcacccgtgatggcccttctgactgtgtcctggctgctgattggttgccttctctgatgttgttattagagggtacacgctggcaggccctggtgcagttgcagcccagtgtggacccaaccaatgccacaggtcttgatggtagagagccagctcctcactttttacagggtctgctgggcttgcttaccccagcaggagagttgggctctgaggaggctctttggggtggtctgctgcgcacagtgggggcccccctctatgctgccttccaggaggggctactgcgagtcactcattctctgcaagatgaggtcttttctattatgggacagccagagcctgatgccagtgggcagtgccagggaggcaaccttcaacagctgcttttatggggcatgcggaacaacctttcttgggacgcccgagcactgggttttctatctggatcaccacctccaccccctgctctcctgcactgcctgagcagaggtgtgcctctgcccagggcttcccagcctgcggctcacatcagccctcgacagcggcgagccatctctgtggaggccctctgcgagaaccactcaggcccagagccaccctacagcatctccaacttctccatctacttgctctgccagcacatcaagcctgccaccccgcggccccctcctaccaccccacggcctcctcctaccaccccacagccccctcctaccactacacagcccattcctgacactacacagccccctcctgtcaccccaaggcctcctcctaccaccccacaaccccctcctagcacagctgtcatctgccagacagctgtatggtacgcagtctcgtgggcaccaggtgcccgaggttggctccaagcctgccatgatcagtttcctgatcaatttctggatatgatctgcggcaacctctcattttcagccctgtctggccccagtcgtcctttggtaaagcagctctgtgctggcttgctcccaccccccactagctgtccaccaggcctgatccctgtgcccctcaccccagaaatattctggggctgtttcctggagaatgagacactgtgggctgaacggttgtgtgtggaggacagtctgcaggctgtgcccccgaggaaccaggcttgggttcagcatgtgtgtcggggccccaccttggacgccactgattttccaccgtgccgcgttggaccctgtggggaacgctgcccagatgggggcagcttcctgctcatggtctgtgccaatgacactctgtatgaagccttggttcccttctgggcttggctagcaggccaatgcagaattagtcgtggaggaaatgatacttgctttctagaaggcatgctgggccccttgttgccctctctgccccctctgggaccatccccactctgtctggctcctggtccttttctgcttggcatgttatcccagttgccacgctgtcagtcctccgtgccagccctcgcccaccccacgcgcctacattacctcctgcgcctactgaccttccttctgggtccagggactgggggtgccgagacgcaggggatgttaggtcaagccctgctgctctctagtctcccagacaactgttcattctgggatgccttccgcccagagggccggagaagtgtactgaggacagtcggagagtacttgcagcgggaagagccaaccccaccaggcttagactcctccctcagcctcggctctggtatgagcaagatggagcttctgtcctgcttcagtcctgtactgtgggatctactccagagagagaagagcgtttgggccctgaggaccctggtgaaggcctacctgcgcatgcctccagaagaccttcagcagcttgtgctttcagcagagatggaggctgcacagggcttcctgacgctcatgcttcgttcctgggctaagctgaaggttcaaccatccgaggagcaggccatgggccgcctgacagccttgctgctccagcggtacccacgcctcacctcccaactctttatcgacatgtcaccgctcatccccttcctggctgtccctgacctcatgcgcttcccaccgtcccttttggccaacgacagtgtcctggctgccatcagggatcacagctcaggaatgaagcctgaacagaaggaggccctggcaaaacgactgctggcccctgagctgtttggagaagtgcctgattggccccaggagctgctgtgggcagccctgcctctgcttccccatctgcctctggagagctttctccagctcagccctcaccagatccaggccctggaggatagctggccagtagcagatcttgggccgggacacgcccgacatgtgcttcgtagcctagtaaaccagagcatggaggatggggaggagcaggtgctcaggcttgggtccctcgcctgtttcctgagtcctgaggagctacagagtctggtgcccttgagtgatccaatggggcctgtagaacagggtctgctggaatgtgcggccaatgggaccctcagcccagaaggacgggtggcatatgaacttctgggagtgttgcgttcatctggaggaactgtcttaagcccccgagagctgagggtctgggcacctctctttccccagctgggcctccgcttcctgcaggagctctcagagacccagcttagagccatgcttcctgccctacagggagccagtgtcacacctgcccaggctgttctgttgtttggaaggctccttcctaagcatgatctgtccctggaggaactctgctccctgcaccctctcctgccaggtctcagcccccagacactccaggccatccctaagagagttctggttggtgcttgttcctgcctgggccctgaactgtcaaggctttcagcttgccagattgcagctctgctgcagacctttcgggtaaaagatggtgttaaaaatatgggtgcagcaggtgccggctcagccgtgtgcattcctgggcagcccaccacttggccagactgcctgcttcccctgctcccattaaagctgctacagctggacgctgcagctcttctggcaaaccgaagactctatcggcagctgccttggtctgagcaacaggcacagtttctctggaagaaaatgcaagtgcctaccaacctgagcctgaggaatctgcaggctctgggcaacttggcaggaggcatgacctgcgagtttctgcagcagatcagctcaatggttgactttcttgatgtggtacacatgctctaccagctgcccactggtgttcgagagagcctgcgggcctgtatctggacagagctacagcggaggatgacaatgccagagccagagctgaccaccctagggccagaactgagtgaacttgacacaaagctactcctggacttgccgatccagctgatggacagattgtccaatgattccattatgttggtggtggagatggtccaaggcgctccagagcagctgctggcactgaccccactccaccagacagccttggcagagcgagcacttaaaaacctggctccaaaggagaccccaatctccaaagaagtgctggagacactgggccccttggttggattcctgggaatagagagcacgcgacggatccctttacccattctactgtctcatctcagtcagctgcagggcttctgcctaggagagacatttgccacagagctgggatggctgctgttgcaggagcctgttcttggaaaaccagaattgtggagccaggatgaaatagagcaagctggacgcctagtattcactctgtctgctgaggctatttcctcgatccccagggaggctttgggcccagagacactggagaggcttctgggaaagcatcaaagctgggagcagagcagagtgggccatctgtgtggggagtcacagcttgcccacaagaaagcagctctggtagctgggattgtgcatccagctgctgagggtctccaagagcctgtaccaaactgtgcagacatacggggaaccttcccagcggcctggtctgcgacacaaatctcagagatggaactctcagactttgaagactgcctgtcactatttgctggagatccaggacttggtcctgaggaactacgggcagccatgggcaaggccaagcagttgtggggtccccctcgaggattccgtcctgagcagatcttgcagctgggccgtctcctgataggtctaggagaacgggaactgcaggagcttaccttggtggactggggtgtgctgagcagcctggggcaaatagatggctggagttccatgcagctccgagccgtggtctccagtttcctaaggcagagtggtcggcatgtgagccacctggacttcatttatctgacagcactgggttacacagtctgtggattgcgaccagaggagttacagcacatcagcagttgggagtttagccaagcagctctcttcctgggtagcttgcatctcccgtgctctgaggaacagctggaagttctggcctatctccttgtgttgcctggtggctttggcccagtcagtaactgggggcctgagatcttcactgaaattggcacaatagcagctggcatcccagacctggctctttcagcattactgcggggacagatccaaggcctgactcctcttgccatttctgtcattcctgctcccaagtttgcagtggtcttcaaccccatccagttatctagtctcaccaggggtcaggccgtagctgttactcctgaacagctggcctatctgagtcctgagcagcggcgagcagttgcatgggcccaacacgaagggaaggagatcccagagcagctgggtcgaaactcagcctggggtctctacgactggttccaagcctcctgggccctggcattgcccgtcagcatttttggccacctattatga

SEQ ID NO: 8 - protein, 92 aa, Artificial Sequence
  Synthetic Construct
CLSYDTEILTVEYGFLPIGKIVEERIECTVYTVDKNGFVYTQPIAQWHNRGEQEVFEYCLEDGSIIRATKDHKFMTTDGQMLPIDEIFERGL

SEQ ID NO: 9 - protein, 35 aa, Artificial Sequence
  Synthetic Construct
VKIISRKSLGTQNVYDIGVEKDHNFLLKNGLVASN

SEQ ID NO: 10 - protein, 101 aa, Artificial Sequence
  Synthetic Construct
CLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLP

SEQ ID NO: 11 - protein, 36 aa, Artificial Sequence
  Synthetic Construct
MVKIISRKSLGTQNVYDIGVEKDHNFLLKNGLVASN

SEQ ID NO: 12 - protein, 35 aa, Artificial Sequence
  Synthetic Construct
VKIISRKSLGTQNVYDIGVGEPHNFLLKNGLVASN

SEQ ID NO: 13 - protein, 102 aa, Artificial Sequence
  Synthetic Construct
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 14 - protein, 36 aa, Artificial Sequence
  Synthetic Construct
MIKIATRKYLGKQNVYDIGVERDHNFALKNGFIASN

SEQ ID NO: 15 - protein, 101 aa, Artificial Sequence; identical to SEQ ID NO: 10
  Synthetic Construct

SEQ ID NO: 16 - protein, 53 aa, Artificial Sequence
  Synthetic Construct
MKRTADGSEFESPKKKRKVKIISRKSLGTQNVYDIGVEKDHNFLLKNGLVASN

SEQ ID NO: 17 - protein, 12 aa, Artificial Sequence
  Synthetic Construct
CFSGDTLVALTD

SEQ ID NO: 18 - protein, 11 aa, Artificial Sequence
  Synthetic Construct
CLAGDTLITLA

SEQ ID NO: 19 - protein, 10 aa, Artificial Sequence
  Synthetic Construct
CLQNGTRLLR

SEQ ID NO: 20 - protein, 11 aa, Artificial Sequence
  Synthetic Construct
CLTGDSQVLTR

SEQ ID NO: 21 - protein, 11 aa, Artificial Sequence
  Synthetic Construct
CLTYETEIMTV

SEQ ID NO: 22 - protein, 12 aa, Artificial Sequence
  Synthetic Construct
CLSGNTKVRFRY

SEQ ID NO: 23 - protein, 6 aa, Artificial Sequence
  Synthetic Construct
GVFVHN

SEQ ID NO: 24 - protein, 6 aa, Artificial Sequence
  Synthetic Construct
GLLVHN

SEQ ID NO: 25 - protein, 6 aa, Artificial Sequence
  Synthetic Construct
GLIASN

SEQ ID NO: 26 - protein, 6 aa, Artificial Sequence
  Synthetic Construct
GLVVHN

SEQ ID NO: 27 - protein, 102 aa, Artificial Sequence; identical to SEQ ID NO: 13
  Synthetic Construct

SEQ ID NO: 28 - protein, 35 aa, Artificial Sequence
  Synthetic Construct
IKIATRKYLGKQNVYDIGVERDHNFALKNGFIASN

SEQ ID NO: 29 - protein, 106 aa, Artificial Sequence
  Synthetic Construct
CLAGDTLITLADGRRVPIRELVSQQNFSVWALNPQTYRLERARVSRAFCTGIKPVYRLTTRLGRSIRATANHRFLTPQGWKRVDELQPGDYLALPRRIPTASTPTL

SEQ ID NO: 30 - protein, 51 aa, Artificial Sequence
  Synthetic Construct
AAACPELRQLAQSDVYWDPIVSIEPDGVEEVFDLTVPGPHNFVANDIIAHN

SEQ ID NO: 31 - protein, 102 aa, Artificial Sequence
  Synthetic Construct
CLSYDTEILTVEYGILPIGKIVEKRIECTVYSVDNNGNIYTQPVAQWHDRGEQEVFEYCLEDGSLIRATKDHKFMTVDGQMMPIDEIFERELDLMRVDNLPN

SEQ ID NO: 32 - protein, 35 aa, Artificial Sequence
  Synthetic Construct
VKVIGRRSLGVQRIFDIGLPQYHNFLLANGAIAAN

SEQ ID NO: 33 - protein, 101 aa, Artificial Sequence; identical to SEQ ID NO: 10
  Synthetic Construct

SEQ ID NO: 34 - protein, 35 aa, Artificial Sequence; identical to SEQ ID NO: 9
  Synthetic Construct

SEQ ID NO: 35 - protein, 101 aa, Artificial Sequence; identical to SEQ ID NO: 10
  Synthetic Construct

SEQ ID NO: 36 - protein, 52 aa, Artificial Sequence
  Synthetic Construct
KRTADGSEFESPKKKRKVKIISRKSLGTQNVYDIGVEKDHNFLLKNGLVASN

SEQ ID NO: 37 - protein, 123 aa, Artificial Sequence
  Synthetic Construct
CLSFGTEILTVEYGPLPIGKIVSEEINCSVYSVDPEGRVYTQAIAQWHDRGEQEVLEYELEDGSVIRATSDHRFLTTDYQLLAIEEIFARQLDLLTLENIKQTEEALDNHRLPFPLLDAGTIK

SEQ ID NO: 38 - protein, 36 aa, Artificial Sequence
  Synthetic Construct
VKVIGRRSLGVQRIFDIGLPQDHNFLLANGAIAANC

SEQ ID NO: 39 - protein, 97 aa, Artificial Sequence
  Synthetic Construct
CLSYDTEILTVEYGFLPIGKIVEERIECTVYTVDKNGFVYTQPIAQWHNRGEQEVFEYCLEDGSIIRATKDHKFMTTDGQMLPIDEIFERGLDLQVD

SEQ ID NO: 40 - protein, 38 aa, Artificial Sequence
  Synthetic Construct
GLPVKIISRKSLGTQNVYDIGVEKDHNFLLKNGLVASN

SEQ ID NO: 41 - DNA, 306 bp, Artificial Sequence
  Synthetic Construct
tgcctgagctacgagaccgagatcctgaccgtggagtacggcctgctgcccatcggcaagatcgtggagaagagaatcgagtgcaccgtgtacagcgtggacaacaacggcaacatctacacccagcccgtggcccagtggcacgacagaggcgagcaggaggtgttcgagtactgcctggaggacggcagcctgatcagagccaccaaggaccacaagttcatgaccgtggacggccagatgctgcccatcgacgagatcttcgagagagagctggacctgatgagagtggacaacctgcccaac

SEQ ID NO: 42 - DNA, 105 bp, Artificial Sequence
  Synthetic Construct
atcaagatcgccacaagaaagtacctgggcaagcagaacgtgtacgacatcggcgtggagagagaccacaacttcgccctgaagaacggcttcatcgccagcaat

SEQ ID NO: 43 - DNA, 7561 bp, Artificial Sequence
  Synthetic Construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccaccggtagcaggtttgttacagaaaccttagttaaggtttgttgagggttttttttctctctctctctcttaattggctgtcccaatccatccttctataaatagaaaagagagacagggagtgtgtgtggtttcattactaaggtaaagacacttgagctacacacacttgatccctgaacatgaaatctaagaggttgaacgatcacagtttcaggactatataaggtggtgaaagaccatctgcttcgtttttctgtttgttcctacaactctttccctccgcttgattttaactctaaattggtgagtagctggtgggctcaccagactccgagatcctcttctctgcacgcactgtattagacttggcacccgggaggattttcacctctgctgcatgggctaatcttccacaagggatctgtggtattgcaatctcgggttgatgcatgacggtgatgttgtgtttatagcatggctaaggtttagctgcctatgatgattggttagggaaggataatttttgctagaagattggactttagggaaaaaaaaccccacttttatttgcttttagaattttaaaagactgggccatgtagctcaggctggtttggagttcattatgtagtcaaggatgctcttggactctttagcatcctcctcctcctctttttcctcctcctccttcttgttcttcttcttgttcttcctcttccccttctcttcccccttctctccctcttcctcctcttcctcctccttcttgttcttcttcctcttccccttctcctctcccccttgtcctcctcttccttctcctcctcctcctcttcttctttctgagtaccaagattgcaagtgtgcacacgatgaccagcttggtctttctttgtcttttttttttaacttcaattttggagtgaattcaagagcaaccatgtagtcaagaggtggctggagtcttttctgtatctgggtttggtttagtactctgccccatcacttaacaggtccttatggccacatcttaaaaaaattctagagatacacggtgtcggtgagtggctgagaatgtgtggtcttcccatttctctgtcaccgtggctcacatcttgtttcctctgttcggccaggtagaaaggcggccgccatggctctgagcctccagccccagctgctccttctcctgtcgctcctgccgcaggaagtgacttcagcccctactgggcctcagtctttggatgctggtctctcccttctgaagtcattcgtagccactctggaccaagctcctcagcgttccctcagccagtcacggttctctgcgttcctggccaacatttcttcatccttccagcttgggaggatgggggagggaccggtgggagagcccccacctctccagccccctgcacttcgacttcatgatttcctcgtgacactgagaggtagcccagactgggagccaatgctagggcttctgggagatgtgctggcactcctgggacaggaacagactccccgggactttttggtgcaccaggcaggtgtactgggtggacttgtagaggcattgttgggagcgttagttcctggaggcccccctgcccccactcgacccccatgcacccgtgatggcccttctgactgtgtcctggctgctgattggttgccttctctgatgttgttattagagggtacacgctggcaggccctggtgcagttgcagcccagtgtggacccaaccaatgccacaggtcttgatggtagagagccagctcctcactttttacagggtctgctgggcttgcttaccccagcaggagagttgggctctgaggaggctctttggggtggtctgctgcgcacagtgggggcccccctctatgctgccttccaggaggggctactgcgagtcactcattctctgcaagatgaggtcttttctattatgggacagccagagcctgatgccagtgggcagtgccagggaggcaaccttcaacagctgcttttatggggcatgcggaacaacctttcttgggacgcccgagcactgggttttctatctggatcaccacctccaccccctgctctcctgcactgcctgagcagaggtgtgcctctgcccagggcttcccagcctgcggctcacatcagccctcgacagcggcgagccatctctgtggaggccctctgcgagaaccactcaggcccagagccaccctacagcatctccaacttctccatctacttgctctgccagcacatcaagcctgccaccccgcggccccctcctaccaccccacggcctcctcctaccaccccacagccccctcctaccactacacagcccattcctgacactacacagccccctcctgtcaccccaaggcctcctcctaccaccccacaaccccctcctagcacagctgtcatctgccagacagctgtatggtacgcagtctcgtgggcaccaggtgcccgaggttggctccaagcctgccatgatcagtttcctgatcaatttctggatatgatctgcggcaacctctcattttcagccctgtctggccccagtcgtcctttggtaaagcagctctgtgctggcttgctcccaccccccactagctgtccaccaggcctgatccctgtgcccctcaccccagaaatattctggggctgtttcctggagaatgagacactgtgggctgaacggttgtgtgtggaggacagtctgcaggctgtgcccccgaggaaccaggcttgggttcagcatgtgtgtcggggccccaccttggacgccactgattttccaccgtgccgcgttggaccctgtggggaacgctgcccagatgggggcagcttcctgctcatggtctgtgccaatgacactctgtatgaagccttggttcccttctgggcttggctagcaggccaatgcagaattagtcgtggaggaaatgatacttgctttctagaaggcatgctgggccccttgttgccctctctgccccctctgggaccatccccactctgtctggctcctggtccttttctgcttggcatgttatcccagttgccacgctgtcagtcctccgtgccagccctcgcccaccccacgcgcctacattacctcctgcgcctactgaccttccttctgggtccagggactgggggtgccgagacgcaggggatgttaggtcaagccctgctgctctctagtctcccagacaactgttcattctgggatgccttccgcccagagggccggagaagtgtactgaggacagtcggagagtacttgcagcgggaagagccaaccccaccaggcttagactcctccctcagcctcggctctggtatgagcaagatggagcttctgtcctgcttcagtcctgtactgtgggatctactccagagagagaagagcgtttgggccctgaggaccctggtgaaggcctacctgcgcatgcctccagaagaccttcagcagcttgtgctttcagcagagatggaggctgcacagggcttcctgacgctcatgcttcgttcctgggctaagctgaaggttcaaccatccgaggagcaggccatgggccgcctgacagccttgctgctccagcggtacccacgcctcacctcccaactctttatcgacatgtcaccgctcatccccttcctggctgtccctgacctcatgcgcttcccaccgtcccttttggccaacgacagtgtcctggctgccatcagggatcacagctcaggaatgaagcctgaacagaaggaggccctggcaaaacgactgctggcccctgagctgtttggagaagtgcctgattggccccaggagctgctgtgggcagccctgcctctgcttccccatctgcctctggagagctttctccagctcagccctcaccagatccaggccctggaggatagctggccagtagcagatcttgggccgggacacgcccgacatgtgcttcgtagcctagtaaaccagagcatggaggatggggaggagcaggtgctcaggcttgggtccctcgcctgtttcctgagtcctgaggagctacagagtctggtgcccttgagtgatccaatggggcctgtagaacagggtctgctggaatgtgcggccaatgggaccctcagcccagaaggacgggtggcatatgaacttctgggagtgttgcgttcatctggaggaactgtcttaagcccccgagagctgagggtctgggcacctctctttccccagctgggcctccgcttcctgcaggagctctcagagacccagcttagagccatgcttcctgccctacagggagccagtgtcacaccctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 44 - DNA, 7206 bp, Artificial Sequence
  Synthetic Construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcgcctcggctctggtatgagcaagatggagcttctgtcctgcttcagtcctgtactgtgggatctactccagagagagaagagcgtttgggccctgaggaccctggtgaaggcctacctgcgcatgcctccagaagaccttcagcagcttgtgctttcagcagagatggaggctgcacagggcttcctgacgctcatgcttcgttcctgggctaagctgaaggttcaaccatccgaggagcaggccatgggccgcctgacagccttgctgctccagcggtacccacgcctcacctcccaactctttatcgacatgtcaccgctcatccccttcctggctgtccctgacctcatgcgcttcccaccgtcccttttggccaacgacagtgtcctggctgccatcagggatcacagctcaggaatgaagcctgaacagaaggaggccctggcaaaacgactgctggcccctgagctgtttggagaagtgcctgattggccccaggagctgctgtgggcagccctgcctctgcttccccatctgcctctggagagctttctccagctcagccctcaccagatccaggccctggaggatagctggccagtagcagatcttgggccgggacacgcccgacatgtgcttcgtagcctagtaaaccagagcatggaggatggggaggagcaggtgctcaggcttgggtccctcgcctgtttcctgagtcctgaggagctacagagtctggtgcccttgagtgatccaatggggcctgtagaacagggtctgctggaatgtgcggccaatgggaccctcagcccagaaggacgggtggcatatgaacttctgggagtgttgcgttcatctggaggaactgtcttaagcccccgagagctgagggtctgggcacctctctttccccagctgggcctccgcttcctgcaggagctctcagagacccagcttagagccatgcttcctgccctacagggagccagtgtcacacctgcccaggctgttctgttgtttggaaggctccttcctaagcatgatctgtccctggaggaactctgctccctgcaccctctcctgccaggtctcagcccccagacactccaggccatccctaagagagttctggttggtgcttgttcctgcctgggccctgaactgtcaaggctttcagcttgccagattgcagctctgctgcagacctttcgggtaaaagatggtgttaaaaatatgggtgcagcaggtgccggctcagccgtgtgcattcctgggcagcccaccacttggccagactgcctgcttcccctgctcccattaaagctgctacagctggacgctgcagctcttctggcaaaccgaagactctatcggcagctgccttggtctgagcaacaggcacagtttctctggaagaaaatgcaagtgcctaccaacctgagcctgaggaatctgcaggctctgggcaacttggcaggaggcatgacctgcgagtttctgcagcagatcagctcaatggttgactttcttgatgtggtacacatgctctaccagctgcccactggtgttcgagagagcctgcgggcctgtatctggacagagctacagcggaggatgacaatgccagagccagagctgaccaccctagggccagaactgagtgaacttgacacaaagctactcctggacttgccgatccagctgatggacagattgtccaatgattccattatgttggtggtggagatggtccaaggcgctccagagcagctgctggcactgaccccactccaccagacagccttggcagagcgagcacttaaaaacctggctccaaaggagaccccaatctccaaagaagtgctggagacactgggccccttggttggattcctgggaatagagagcacgcgacggatccctttacccattctactgtctcatctcagtcagctgcagggcttctgcctaggagagacatttgccacagagctgggatggctgctgttgcaggagcctgttcttggaaaaccagaattgtggagccaggatgaaatagagcaagctggacgcctagtattcactctgtctgctgaggctatttcctcgatccccagggaggctttgggcccagagacactggagaggcttctgggaaagcatcaaagctgggagcagagcagagtgggccatctgtgtggggagtcacagcttgcccacaagaaagcagctctggtagctgggattgtgcatccagctgctgagggtctccaagagcctgtaccaaactgtgcagacatacggggaaccttcccagcggcctggtctgcgacacaaatctcagagatggaactctcagactttgaagactgcctgtcactatttgctggagatccaggacttggtcctgaggaactacgggcagccatgggcaaggccaagcagttgtggggtccccctcgaggattccgtcctgagcagatcttgcagctgggccgtctcctgataggtctaggagaacgggaactgcaggagcttaccttggtggactggggtgtgctgagcagcctggggcaaatagatggctggagttccatgcagctccgagccgtggtctccagtttcctaaggcagagtggtcggcatgtgagccacctggacttcatttatctgacagcactgggttacacagtctgtggattgcgaccagaggagttacagcacatcagcagttgggagtttagccaagcagctctcttcctgggtagcttgcatctcccgtgctctgaggaacagctggaagttctggcctatctccttgtgttgcctggtggctttggcccagtcagtaactgggggcctgagatcttcactgaaattggcacaatagcagctggcatcccagacctggctctttcagcattactgcggggacagatccaaggcctgactcctcttgccatttctgtcattcctgctcccaagtttgcagtggtcttcaaccccatccagttatctagtctcaccaggggtcaggccgtagctgttactcctgaacagctggcctatctgagtcctgagcagcggcgagcagttgcatgggcccaacacgaagggaaggagatcccagagcagctgggtcgaaactcagcctggggtctctacgactggttccaagcctcctgggccctggcattgcccgtcagcatttttggccacctattatgataataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 45 - DNA, 7447 bp, Artificial Sequence
  Synthetic Construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccaccggtagcaggtttgttacagaaaccttagttaaggtttgttgagggttttttttctctctctctctcttaattggctgtcccaatccatccttctataaatagaaaagagagacagggagtgtgtgtggtttcattactaaggtaaagacacttgagctacacacacttgatccctgaacatgaaatctaagaggttgaacgatcacagtttcaggactatataaggtggtgaaagaccatctgcttcgtttttctgtttgttcctacaactctttccctccgcttgattttaactctaaattggtgagtagctggtgggctcaccagactccgagatcctcttctctgcacgcactgtattagacttggcacccgggaggattttcacctctgctgcatgggctaatcttccacaagggatctgtggtattgcaatctcgggttgatgcatgacggtgatgttgtgtttatagcatggctaaggtttagctgcctatgatgattggttagggaaggataatttttgctagaagattggactttagggaaaaaaaaccccacttttatttgcttttagaattttaaaagactgggccatgtagctcaggctggtttggagttcattatgtagtcaaggatgctcttggactctttagcatcctcctcctcctctttttcctcctcctccttcttgttcttcttcttgttcttcctcttccccttctcttcccccttctctccctcttcctcctcttcctcctccttcttgttcttcttcctcttccccttctcctctcccccttgtcctcctcttccttctcctcctcctcctcttcttctttctgagtaccaagattgcaagtgtgcacacgatgaccagcttggtctttctttgtcttttttttttaacttcaattttggagtgaattcaagagcaaccatgtagtcaagaggtggctggagtcttttctgtatctgggtttggtttagtactctgccccatcacttaacaggtccttatggccacatcttaaaaaaattctagagatacacggtgtcggtgagtggctgagaatgtgtggtcttcccatttctctgtcaccgtggctcacatcttgtttcctctgttcggccaggtagaaaggcggccgccaccatggctctgagcctccagccccagctgctccttctcctgtcgctcctgccgcaggaagtgacttcagcccctactgggcctcagtctttggatgctggtctctcccttctgaagtcattcgtagccactctggaccaagctcctcagcgttccctcagccagtcacggttctctgcgttcctggccaacatttcttcatccttccagcttgggaggatgggggagggaccggtgggagagcccccacctctccagccccctgcacttcgacttcatgatttcctcgtgacactgagaggtagcccagactgggagccaatgctagggcttctgggagatgtgctggcactcctgggacaggaacagactccccgggactttttggtgcaccaggcaggtgtactgggtggacttgtagaggcattgttgggagcgttagttcctggaggcccccctgcccccactcgacccccatgcacccgtgatggcccttctgactgtgtcctggctgctgattggttgccttctctgatgttgttattagagggtacacgctggcaggccctggtgcagttgcagcccagtgtggacccaaccaatgccacaggtcttgatggtagagagccagctcctcactttttacagggtctgctgggcttgcttaccccagcaggagagttgggctctgaggaggctctttggggtggtctgctgcgcacagtgggggcccccctctatgctgccttccaggaggggctactgcgagtcactcattctctgcaagatgaggtcttttctattatgggacagccagagcctgatgccagtgggcagtgccagggaggcaaccttcaacagctgcttttatggggcatgcggaacaacctttcttgggacgcccgagcactgggttttctatctggatcaccacctccaccccctgctctcctgcactgcctgagcagaggtgtgcctctgcccagggcttcccagcctgcggctcacatcagccctcgacagcggcgagccatctctgtggaggccctctgcgagaaccactcaggcccagagccaccctacagcatctccaacttctccatctacttgctctgccagcacatcaagcctgccaccccgcggccccctcctaccaccccacggcctcctcctaccaccccacagccccctcctaccactacacagcccattcctgacactacacagccccctcctgtcaccccaaggcctcctcctaccaccccacaaccccctcctagcacagctgtcatctgccagacagctgtatggtacgcagtctcgtgggcaccaggtgcccgaggttggctccaagcctgccatgatcagtttcctgatcaatttctggatatgatctgcggcaacctctcattttcagccctgtctggccccagtcgtcctttggtaaagcagctctgtgctggcttgctcccaccccccactagctgtccaccaggcctgatccctgtgcccctcaccccagaaatattctggggctgtttcctggagaatgagacactgtgggctgaacggttgtgtgtggaggacagtctgcaggctgtgcccccgaggaaccaggcttgggttcagcatgtgtgtcggggccccaccttggacgccactgattttccaccgtgccgcgttggaccctgtggggaacgctgcccagatgggggcagcttcctgctcatggtctgtgccaatgacactctgtatgaagccttggttcccttctgggcttggctagcaggccaatgcagaattagtcgtggaggaaatgatacttgctttctagaaggcatgctgggccccttgttgccctctctgccccctctgggaccatccccactctgtctggctcctggtccttttctgcttggcatgttatcccagttgccacgctgtcagtcctccgtgccagccctcgcccaccccacgcgcctacattacctcctgcgcctactgaccttccttctgggtccagggactgggggtgccgagacgcaggggatgttaggtcaagccctgctgctctctagtctcccagacaactgttcattctgggatgccttccgcccagagggccggagaagtgtactgaggacagtcggagagtacttgcagcgggaagagccaaccccaccaggcttagactcctccctcagcctcggctctggtatgagcaagatggagcttctgtcctgcttcagtcctgtactgtgggatctactccagagagagaagagcgtttgggccctgaggaccctggtgaaggcctacctgcgcatgcctccagaagaccttcagcagcttgtgctttcagcagagatggaggctgcacagggcttcctgacgctcatgcttcgttcctgggctaagctgaaggttcaaccatccgaggagcaggccatgggccgcctgacagccttgctgctccagcggtacccacgcctcacctcccaactctttatcgacatgtcaccgctcatccccttcctggctgtccctgacctcatgcgcttcccaccgtcccttttggccaacgacagtgtcctggctgccatcagggatcacagctcaggaatgaagcctgaacagaaggaggccctggcaaaacgactgctggcccctgagctgtttggagaagtgcctgattggccccaggtaagtatcaaggttacaagacaggtttaaggagaccaatagaaactgggcttgtcgagacagagaagactcttgcgtttctgagctagcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttcgtcgacctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 46 - DNA, 6573 bp, Artificial Sequence
  Synthetic Construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgcccaattggcttcgaattctagcggccgcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccttaagcgacgcatgctcgcgataggcacctattggtcttactgacatccactttgcctttctctccacaggagctgctgtgggcagccctgcctctgcttccccatctgcctctggagagctttctccagctcagccctcaccagatccaggccctggaggatagctggccagtagcagatcttgggccgggacacgcccgacatgtgcttcgtagcctagtaaaccagagcatggaggatggggaggagcaggtgctcaggcttgggtccctcgcctgtttcctgagtcctgaggagctacagagtctggtgcccttgagtgatccaatggggcctgtagaacagggtctgctggaatgtgcggccaatgggaccctcagcccagaaggacgggtggcatatgaacttctgggagtgttgcgttcatctggaggaactgtcttaagcccccgagagctgagggtctgggcacctctctttccccagctgggcctccgcttcctgcaggagctctcagagacccagcttagagccatgcttcctgccctacagggagccagtgtcacacctgcccaggctgttctgttgtttggaaggctccttcctaagcatgatctgtccctggaggaactctgctccctgcaccctctcctgccaggtctcagcccccagacactccaggccatccctaagagagttctggttggtgcttgttcctgcctgggccctgaactgtcaaggctttcagcttgccagattgcagctctgctgcagacctttcgggtaaaagatggtgttaaaaatatgggtgcagcaggtgccggctcagccgtgtgcattcctgggcagcccaccacttggccagactgcctgcttcccctgctcccattaaagctgctacagctggacgctgcagctcttctggcaaaccgaagactctatcggcagctgccttggtctgagcaacaggcacagtttctctggaagaaaatgcaagtgcctaccaacctgagcctgaggaatctgcaggctctgggcaacttggcaggaggcatgacctgcgagtttctgcagcagatcagctcaatggttgactttcttgatgtggtacacatgctctaccagctgcccactggtgttcgagagagcctgcgggcctgtatctggacagagctacagcggaggatgacaatgccagagccagagctgaccaccctagggccagaactgagtgaacttgacacaaagctactcctggacttgccgatccagctgatggacagattgtccaatgattccattatgttggtggtggagatggtccaaggcgctccagagcagctgctggcactgaccccactccaccagacagccttggcagagcgagcacttaaaaacctggctccaaaggagaccccaatctccaaagaagtgctggagacactgggccccttggttggattcctgggaatagagagcacgcgacggatccctttacccattctactgtctcatctcagtcagctgcagggcttctgcctaggagagacatttgccacagagctgggatggctgctgttgcaggagcctgttcttggaaaaccagaattgtggagccaggatgaaatagagcaagctggacgcctagtattcactctgtctgctgaggctatttcctcgatccccagggaggctttgggcccagagacactggagaggcttctgggaaagcatcaaagctgggagcagagcagagtgggccatctgtgtggggagtcacagcttgcccacaagaaagcagctctggtagctgggattgtgcatccagctgctgagggtctccaagagcctgtaccaaactgtgcagacatacggggaaccttcccagcggcctggtctgcgacacaaatctcagagatggaactctcagactttgaagactgcctgtcactatttgctggagatccaggacttggtcctgaggaactacgggcagccatgggcaaggccaagcagttgtggggtccccctcgaggattccgtcctgagcagatcttgcagctgggccgtctcctgataggtctaggagaacgggaactgcaggagcttaccttggtggactggggtgtgctgagcagcctggggcaaatagatggctggagttccatgcagctccgagccgtggtctccagtttcctaaggcagagtggtcggcatgtgagccacctggacttcatttatctgacagcactgggttacacagtctgtggattgcgaccagaggagttacagcacatcagcagttgggagtttagccaagcagctctcttcctgggtagcttgcatctcccgtgctctgaggaacagctggaagttctggcctatctccttgtgttgcctggtggctttggcccagtcagtaactgggggcctgagatcttcactgaaattggcacaatagcagctggcatcccagacctggctctttcagcattactgcggggacagatccaaggcctgactcctcttgccatttctgtcattcctgctcccaagtttgcagtggtcttcaaccccatccagttatctagtctcaccaggggtcaggccgtagctgttactcctgaacagctggcctatctgagtcctgagcagcggcgagcagttgcatgggcccaacacgaagggaaggagatcccagagcagctgggtcgaaactcagcctggggtctctacgactggttccaagcctcctgggccctggcattgcccgtcagcatttttggccacctattatgagcggccgcggtaccaagggcggatcctgcatagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 47 - DNA, 865 bp, Homo sapiens
ccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccgtataggaggaccgtgtaggccttcctgtcccgggccttgccagcggccagcccgatgaaggagctccctcgcagggggtagcctccgaaggagaagacgtgggagtggtcggcagtgacgaggctcagcgtgtcctcctcgctggtgagctggcccgccctctcaatggcgtcgtcgaacatgatcgtctcagtcagtgcccggtaagccctgctttcatgatgaccatggtcgatgcgaccaccctccacgaagaggaagaagccgcgggggtgtctgctcagcaggcgcagggcagcctctgtcatctccatcagggaggggtccagtgtggagtctcggtggatctcgtatttcatgtctccaggctcaaagagacccatgagatgggtcacagacgggtccagggaagcctgcatgagctcagtgcggttccacacgtaccgggcaccctggcgttcgccgagccattcctgcaccagattcttcccgtccagcctggtcccaccttggctgtagtcatctgggtactcagggtctggggttcccatgcgaaacatgtactttcggcctcca

SEQ ID NO: 48 - DNA, 437 bp, Homo sapiens
ccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccgtataggaggaccgtgtaggccttcctgtcccgggccttgccagcggccagcccgatgaaggagctccctcgcagggggtagcctccgaaggagaagacgtgggagtggtcggcagtgacgaggctcagcgtgtcctcctcgctggtga

SEQ ID NO: 49 - DNA, 428 bp, Homo sapiens
gctggcccgccctctcaatggcgtcgtcgaacatgatcgtctcagtcagtgcccggtaagccctgctttcatgatgaccatggtcgatgcgaccaccctccacgaagaggaagaagccgcgggggtgtctgctcagcaggcgcagggcagcctctgtcatctccatcagggaggggtccagtgtggagtctcggtggatctcgtatttcatgtctccaggctcaaagagacccatgagatgggtcacagacgggtccagggaagcctgcatgagctcagtgcggttccacacgtaccgggcaccctggcgttcgccgagccattcctgcaccagattcttcccgtccagcctggtcccaccttggctgtagtcatctgggtactcagggtctggggttcccatgcgaaacatgtactttcggcctcca

SEQ ID NO: 50 - DNA, 287 bp, Homo sapiens
ccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttc

SEQ ID NO: 51 - DNA, 290 bp, Homo sapiens
cgtataggaggaccgtgtaggccttcctgtcccgggccttgccagcggccagcccgatgaaggagctccctcgcagggggtagcctccgaaggagaagacgtgggagtggtcggcagtgacgaggctcagcgtgtcctcctcgctggtgagctggcccgccctctcaatggcgtcgtcgaacatgatcgtctcagtcagtgcccggtaagccctgctttcatgatgaccatggtcgatgcgaccaccctccacgaagaggaagaagccgcgggggtgtctgctcagcagg

SEQ ID NO: 52 - DNA, 288 bp, Homo sapiens
cgcagggcagcctctgtcatctccatcagggaggggtccagtgtggagtctcggtggatctcgtatttcatgtctccaggctcaaagagacccatgagatgggtcacagacgggtccagggaagcctgcatgagctcagtgcggttccacacgtaccgggcaccctggcgttcgccgagccattcctgcaccagattcttcccgtccagcctggtcccaccttggctgtagtcatctgggtactcagggtctggggttcccatgcgaaacatgtactttcggcctcca

(19) United States
(12) Patent Application Publication
(10) Pub. No.: US 2024/0216540 A1
(43) Pub. Date: Jul. 4, 2024

(54) COMPOSITIONS AND METHODS FOR TREATING SENSORINEURAL HEARING LOSS USING STEREOCILIN DUAL VECTOR SYSTEMS

(71) Applicant: Decibel Therapeutics, Inc., Boston, MA (US)

(72) Inventors: Joseph BURNS, Newton, MA (US); Martin SCHWANDER, Auburndale, MA (US); XuDong WU, Newton, MA (US); Lars BECKER, Boston, MA (US); Tyler GIBSON, Boston, MA (US); Ning PAN, Newton, MA (US)

(21) Appl. No.: 18/289,028

(22) PCT Filed: May 5, 2022
(86) PCT No.: PCT/US2022/027870

§ 371 Date: Oct. 31, 2023

Related U.S. Application Data
(60) Provisional application No. 63/184,737, filed on May 5, 2021.

(51) Int. Cl. A61K 48/00; C07K 14/47; C12N 15/86

(52) U.S. Cl. CPC A61K 48/0058; A61K 48/0075; C07K 14/47; C07K 14/4728; C12N 15/86; C12N 2750/14122; C12N 2750/14143; C12N 2750/14145; C12N 2800/40; C12N 2830/008; C12N 2840/44

(57) ABSTRACT

The disclosure provides compositions containing polynucleotides that encode a stereocilin protein under regulatory control of an outer hair cell-specific promoter, as well as two-vector systems containing the same, that can be used to promote expression of stereocilin specifically in outer hair cells. Additionally, the compositions described herein may be used for the treatment of subjects having or at risk ofdeveloping hearing loss, such as hearing loss associated with a mutation in stereocilin.

### SEQUENCE LISTING

The instant application contains a Sequence Listing which has been submitted electronically in ASCII format and is hereby incorporated by reference in its entirety. The ASCII copy, created on May 4, 2022, is named 51471-011 W02_Sequence_Listing_5_4_22_ST25 and is 113,251 bytes in size.

### FIELD OF THE INVENTION

Described herein are compositions and methods for treatment of hearing loss, particularly forms of the disease that are associated with mutations in stereocilin (STRC) by way of STRC gene therapy, in which the expression of the STRC gene is under regulatory control of an oncomodulin (OCM) promoter. The disclosure provides two-vector expression systems that include a first nucleic acid vector that contains a polynucleotide encoding an N-terminal portion of a stereocilin protein and a second nucleic acid vector that contains a polynucleotide encoding a C-terminal portion of a stereocilin protein. These vectors can be used to increase the expression of or provide wild-type STRC to a cell or subject, such as a subject suffering from hearing loss (e.g., sensorineural hearing loss).

### BACKGROUND

Sensorineural hearing loss is a type of hearing loss caused by defects in the cells of the inner ear or the neural pathways that project from the inner ear to the brain. Although sensorineural hearing loss is often acquired, and can be caused by noise, infections, head trauma, ototoxic drugs, or aging, there are also congenital forms of sensorineural hearing loss associated with autosomal recessive mutations. One such form of autosomal recessive sensorineural hearing loss is associated with mutation of the stereocilin (STRC) gene. Stereocilin is a large protein encoded by the STRC gene on chromosome 15q15, which contains 29 exons spanning approximately 19 kb of the genome. The STRC gene is tandemly duplicated, where the second copy contains a premature stop codon in exon 20, thereby producing an STRC pseudogene. Previous studies have identified mutations in STRC in families with autosomal recessive non-syndromic sensorineural hearing loss (Verpy et al., Nat. Genet. 29:345-9 (2001)). Stereocilin protein expression is limited to stereocilia in hair bundles of inner ear hair cells. Stereocilin protein is thought to form horizontal top connectors and tectorial membrane-attachment crowns, which are required for the normal functioning of the auditory apparatus (Avan et al., PNAS 116:25948-57 (2019); Verpy et al., J. Comp. Neurol. 519:194-210 (2011)). Mice lacking stereocilin have been shown to exhibit abnormal hair cell bundles with defective cohesion and impaired hearing (Verpy et al., Nature 456:255-8 (2008)).

In recent years, efforts to treat hearing loss have increasingly focused on gene therapy as a possible solution; however, the STRC gene is too large to allow for treatment using standard gene therapy approaches. There is a need for new therapeutics to treat STRC-related sensorineural hearing loss.

### SUMMARY OF THE INVENTION

The present invention provides compositions and methods for treating sensorineural hearing loss in a subject, such as a human subject. The compositions and methods of the disclosure pertain to dual vector systems for the delivery of a polynucleotide encoding a stereocilin protein to a subject having or at risk of developing sensorineural hearing loss (e.g., a subject with a mutation in STRC). For example, using the compositions and methods described herein, a first nucleic acid vector and a second nucleic acid vector that each encode a portion of a functional stereocilin protein may be delivered to a subject by way of viral gene therapy. The compositions and methods described herein may also be used to increase expression of a wild-type stereocilin protein in a cochlear hair cell (e.g., an outer hair cell).

In a first aspect, the invention provides a two-vector system comprising (a) a first nucleic acid vector comprising an oncomodulin (OCM) promoter having at least 85% (e.g., at least 86%, 87%, 88%, 89%, 90%, 91%, 92%, 93%, 94%, 95%, 96%, 97%, 98%, 99%, or more) sequence identity to any one of SEQ ID NOs: 1-3 operably linked to a first polynucleotide encoding an N-terminal portion of a stereocilin protein; and (b) a second nucleic acid vector including a second polynucleotide encoding a C-terminal portion of a stereocilin protein.

In some embodiments, the first polynucleotide partially overlaps with the second polynucleotide. In some embodiments, the first polynucleotide and the second polynucleotide have a region of overlap having a length of at least 200 bases (b) (e.g., at least 200 b, 300 b, 400 b, 500 b, 600 b, 700 b, 800 b, 900 b, 1.0 kilobase (kb), 1.1 kb, 1.2 kb, 1.3 kb, 1.4 kb, 1.5 kb or more). In these embodiments, when introduced into a mammalian cell, the first and second nucleic acid vectors undergo homologous recombination to form a recombined polynucleotide that encodes a full-length stereocilin protein. In some embodiments, the first nucleic acid vector includes a polynucleotide including the sequence of nucleotides 225 to 4574 of SEQ ID NO: 43. In some embodiments, the second nucleic acid vector includes a polynucleotide including the sequence of nucleotides 211 to 4219 of SEQ ID NO: 44.

In some embodiments, the first nucleic acid vector includes a splice donor signal sequence positioned at the 3′ end of the first polynucleotide and the second nucleic acid vector includes a splice acceptor signal sequence positioned 5′ of the second polynucleotide. In some embodiments, the first and second polynucleotides do not overlap.

In some embodiments, the first nucleic acid vector includes a splice donor signal sequence positioned at the 3′ end of the first polynucleotide and a first recombinogenic region positioned 3′ of the splice donor signal sequence and the second nucleic acid vector includes a second recombinogenic region, a splice acceptor signal sequence 3′ of the recombinogenic region, and the second polynucleotide 3′ of the splice acceptor signal sequence. In some embodiments, the first and second polynucleotides do not overlap. In some embodiments, the first and second recombinogenic regions are the same. In some embodiments, the first recombinogenic region and the second recombinogenic region is an AP gene fragment. In some embodiments, the AP gene fragment includes or consists of the sequence of any one of SEQ ID NOs: 47-52. In some embodiments, the AP gene fragment includes or consists of the sequence of SEQ ID NO: 50. In some embodiments, the first nucleic acid vector further includes a degradation signal sequence positioned 3′ of the recombinogenic region; and the second nucleic acid vector further includes a degradation signal sequence positioned between the recombinogenic region and the splice acceptor signal sequence. In some embodiments, the first nucleic acid vector includes a polynucleotide including the sequence of nucleotides 225 to 4454 of SEQ ID NO: 45 and the second nucleic acid vector includes a polynucleotide including the sequence of nucleotides 257 to 3597 of SEQ ID NO: 46.

In some embodiments, the second nucleic acid vector further includes an OCM promoter having at least 85% (e.g., at least 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-3 operably linked to the second polynucleotide, wherein the promoter is positioned 5′ of the second polynucleotide. In some embodiments, the OCM promoter in the second nucleic acid vector is the same (i.e., has the same nucleotide sequence) as the OCM promoter in the first nucleic acid vector. In some embodiments, the OCM promoter in the second nucleic acid vector has a different nucleotide sequence than the OCM promoter in the first nucleic acid vector.

In some embodiments, the first nucleic acid vector further includes a polynucleotide encoding an N-terminal intein (N-intein) positioned 3′ of the first polynucleotide. In some embodiments, the second nucleic acid vector further includes a polynucleotide encoding a C-terminal intein (C-intein) positioned between the OCM promoter and the second polynucleotide. In some embodiments, the N-intein and C-intein are components of a split intein trans-splicing system.

In some embodiments, the first and/or second vectors include an intein degradation signal. In some embodiments, the degradation signal is an N-degron and/or a C-degron. In some embodiments, the N-degron and/or the C-degron are independently a CL1, PB29, SMN, CIITA, or ODC degron. In some embodiments, the degradation signal is an E. coli dihydrofolate reductase (ecDHFR) degradation signal. In some embodiments the degradation signal is an FKBP12 degradation domain (Banaszynski et al., Cell 126:995-1004, 2006). In some embodiments the degradation signal is a PEST degradation domain (Rechsteiner and Rogers, Trends Biochem Sci. 21:267-271, 1996). In some embodiments the degradation signal is a UbR tag ubiquitination signal (Chassin et al., Nat Commun. 10:2013, 2019). In some embodiments the degradation signal is a destabilized mutation of human ELRBD (Miyazaki et al., J. Am. Chem. Soc., 134:3942-3945, 2012).

In some embodiments, the first and second vectors, when introduced into a mammalian cell, produce a first and second fusion protein, respectively, wherein the first fusion protein includes the N-terminal portion of stereocilin and the N-intein positioned 3′ thereto, and wherein the second fusion protein includes the C-intein and the C-terminal portion of stereocilin positioned 3′ thereto. In some embodiments, the C-terminus of the N-intein of the first fusion protein and the N-terminus of the C-intein of the second fusion protein are capable of forming a peptide bond, thereby producing a polypeptide including, from N-terminus to C-terminus, the N-terminal portion of stereocilin, N-intein, C-intein, and the C-terminal portion of stereocilin, wherein the bound N-intein and C-intein are capable of self-excising and ligating the C-terminus of the N-terminal portion of stereocilin and the N-terminus of the C-terminal portion of stereocilin, thereby producing a full-length stereocilin protein.

In some embodiments, the split intein trans-splicing system is derived from a DnaEgene of one or more bacteria. In some embodiments, the one or more bacteria are selected from the group consisting of Nostoc punctiforme (Npu), Synechocystis sp. PCC6803 (Ssp), Fischerella sp. PCC9605 (Fsp), Scytonema tolypothrichoides (Sto), Cyanobacteria bacterium SW_9_47_5, Nodularia spumigena (Nsp), Nostoc flagelliforme (Nfl), Crocosphaera watsonii (Cwa) WH8502, Chroococcidiopsis cubana (Ccu) CCALA043, Trichodesmium erythraeum (Ter), Rhodothermus marinus (Rma), Saccharomyces cerevisiae (Sce), Saccharomyces castellii (Sca), Saccharomyces unisporus (Sun), Zygosaccharomyces bisporus (Zbi), Torulaspora pretoriensis (Tpr), Mycobacteria tuberculosis (Mtu), Mycobacterium leprae (Mle), Mycobacterium smegmatis (Msm), Pyrococcus abyssi (Pab), Pyrococcus horikoshii (Pho), Coxiella burnetti (Cbu), Coxiella neoformans (Cne), Coxiella gattii (Cga), Histoplasma capsulatum (Hca), and Porphyra purpurea chloroplast (Ppu). In some embodiments, the split intein trans-splicing system is derived from multiple sequence alignment studies of DnaE for identifying a consensus design (e.g., Cfa) to engineer a split intein with desirable stability and activity.

In some embodiments, the N-intein has a sequence of any one of SEQ ID NOs: 8, 10, 13, 15, 17-22, 27, 29, 31, 33, 35, 37, and 39, and the C-intein has a sequence of any one of SEQ ID NOs: 9, 11, 12, 14, 16, 23-26, 28, 30, 32, 34, 36, 38, and 40. In some embodiments, the N-intein has the sequence of SEQ ID NO: 8 and the C-intein has the sequence of SEQ ID NO: 9. In some embodiments, the N-intein has the sequence of SEQ ID NO: 8 and the C-intein has the sequence of SEQ ID NO: 11. In some embodiments, the N-intein has the sequence of SEQ ID NO: 8 and the C-intein has the sequence of SEQ ID NO: 12. In some embodiments, the N-intein has the sequence of SEQ ID NO: 10 and the C-intein has the sequence of SEQ ID NO: 9. In some embodiments, the N-intein has the sequence of SEQ ID NO: 10 and the C-intein has the sequence of SEQ ID NO: 11. In some embodiments, the N-intein has the sequence of SEQ ID NO: 10 and the C-intein has the sequence of SEQ ID NO: 12. In some embodiments, the N-intein has the sequence of SEQ ID NO: 13 and the C-intein has the sequence of SEQ ID NO: 14. In some embodiments, the N-intein has the sequence of SEQ ID NO: 15 and the C-intein has the sequence of SEQ ID NO: 16. In some embodiments, the N-intein has the sequence of SEQ ID NO: 17 and the C-intein has the sequence of SEQ ID NO: 23. In some embodiments, the N-intein has the sequence of SEQ ID NO: 20 and the C-intein has the sequence of SEQ ID NO: 24. In some embodiments, the N-intein has the sequence of SEQ ID NO: 21 and the C-intein has the sequence of SEQ ID NO: 25. In some embodiments, the N-intein has the sequence of SEQ ID NO: 22 and the C-intein has the sequence of SEQ ID NO: 26. In some embodiments, the N-intein has the sequence of SEQ ID NO: 27 and the C-intein has the sequence of SEQ ID NO: 28. In some embodiments, the N-intein has the sequence of SEQ ID NO: 29 and the C-intein has the sequence of SEQ ID NO: 30. In some embodiments, the N-intein has the sequence of SEQ ID NO: 31 and the C-intein has the sequence of SEQ ID NO: 32. In some embodiments, the N-intein has the sequence of SEQ ID NO: 33 and the C-intein has the sequence of SEQ ID NO: 34. In some embodiments, the N-intein has the sequence of SEQ ID NO: 35 and the C-intein has the sequence of SEQ ID NO: 36. In some embodiments, the N-intein has the sequence of SEQ ID NO: 37 and the C-intein has the sequence of SEQ ID NO: 38. In some embodiments, the N-intein has the sequence of SEQ ID NO: 39 and the C-intein has the sequence of SEQ ID NO: 40. In some embodiments, the N-intein has the sequence of any one of SEQ ID NOs: 17-22 and the C-intein has the sequence of any one of SEQ ID NOs: 23-26.

In some embodiments, the split intein trans-splicing system includes one or more inteins that perform protein trans-splicing only upon contact with a ligand. In some embodiments, the ligand is selected from the group consisting of 4-hydroxytamoxifen, a peptide, a protein, a polynucleotide, an amino acid, or a nucleotide.

In some embodiments, the first nucleic acid vector further includes a polynucleotide encoding a signal peptide. In some embodiments, the polynucleotide encoding a signal peptide is placed 5′ of the polynucleotide encoding the N-terminal portion of the stereocilin protein. In some embodiments, the polynucleotide encoding a signal peptide is placed 3′ of the polynucleotide encoding the N-terminal portion of the stereocilin protein. In some embodiments, the second nucleic acid vector further includes a polynucleotide encoding a signal peptide. In some embodiments, the polynucleotide encoding a signal peptide is placed 5′ of the polynucleotide encoding the C-terminal portion of the stereocilin protein. In some embodiments, the polynucleotide encoding a signal peptide is placed 3′ of the polynucleotide encoding the C-terminal portion of the stereocilin protein.

In some embodiments, neither the first nor the second polynucleotide encodes a full-length stereocilin protein. In some embodiments, each of the first and second polynucleotides encode about half of the stereocilin protein sequence.

In some embodiments, the second nucleic acid vector further includes a poly(A) sequence 3′ of the second polynucleotide.

In some embodiments, the first and second nucleic acid vectors do not include STRC untranslated regions (UTRs). In some embodiments, the first and second nucleic acid vectors include STRC UTRs. In some embodiments, the first nucleic acid vector includes a 5′ STRC UTR 5′ of the first polynucleotide. In some embodiments, the second nucleic acid vector includes a 3′ STRC UTR 3′ of the second polynucleotide.

In some embodiments, the first and second polynucleotides that encode the stereocilin protein do not include introns (e.g., the first and second polynucleotides are portions of STRC cDNA). In some embodiments, the first and second polynucleotides that encode the stereocilin protein include introns.

In some embodiments, the OCM promoter has at least 85% (e.g., at least 86%, 87%, 88%, 89%, 90%, 91%, 92%, 93%, 94%, 95%, 96%, 97%, 98%, 99%, or more) sequence identity to SEQ ID NO: 1. In some embodiments, the OCM promoter has the sequence of SEQ ID NO: 1. In some embodiments, the OCM promoter has at least 85% (e.g., at least 86%, 87%, 88%, 89%, 90%, 91%, 92%, 93%, 94%, 95%, 96%, 97%, 98%, 99%, or more) sequence identity to SEQ ID NO: 2. In some embodiments, the OCM promoter has the sequence of SEQ ID NO: 2. In some embodiments, the OCM promoter has at least 85% (e.g., at least 86%, 87%, 88%, 89%, 90%, 91%, 92%, 93%, 94%, 95%, 96%, 97%, 98%, 99%, or more) sequence identity to SEQ ID NO: 3. In some embodiments, the OCM promoter has the sequence of SEQ ID NO: 3.

In some embodiments, the two-vector system is capable of directing cochlear outer hair cell (OHC)-specific expression of a full-length stereocilin protein in a mammalian OHC. In some embodiments, the mammalian OHC is a human OHC. In some embodiments, the mammalian OHC is a murine OHC.

In some embodiments, the stereocilin protein is a human stereocilin protein having at least 85% (e.g., at least 86%, 87%, 88%, 89%, 90%, 91%, 92%, 93%, 94%, 95%, 96%, 97%, 98%, 99%, or more) sequence identity to SEQ ID NO: 4. In some embodiments, the stereocilin protein has the sequence of SEQ ID NO: 4. In some embodiments, the human stereocilin protein is encoded by a polynucleotide having at least 85% (e.g., at least 86%, 87%, 88%, 89%, 90%, 91%, 92%, 93%, 94%, 95%, 96%, 97%, 98%, 99%, or more) sequence identity to SEQ ID NO: 6. In some embodiments, the polynucleotide that has at least 85% (e.g., at least 85%, 86%, 87%, 88%, 89%, 90%, 91%, 92%, 93%, 94%, 95%, 96%, 97%, 98%, 99%, or more) sequence identity to SEQ ID NO: 6 encodes the stereocilin protein of SEQ ID NO: 4.

In some embodiments, the human stereocilin protein is encoded by a polynucleotide having the sequence of SEQ ID NO: 6. In some embodiments, the STRC protein is a murine stereocilin protein having at least 85% (e.g., at least 86%, 87%, 88%, 89%, 90%, 91%, 92%, 93%, 94%, 95%, 96%, 97%, 98%, 99%, or more) sequence identity to SEQ ID NO: 5. In some embodiments, the murine stereocilin protein has the sequence of SEQ ID NO: 5. In some embodiments, the murine stereocilin protein is encoded by a polynucleotide having at least 85% (e.g., at least 86%, 87%, 88%, 89%, 90%, 91%, 92%, 93%, 94%, 95%, 96%, 97%, 98%, 99%, or more) sequence identity to SEQ ID NO: 7. In some embodiments, the polynucleotide that has at least 85% (e.g., at least 85%, 86%, 87%, 88%, 89%, 90%, 91%, 92%, 93%, 94%, 95%, 96%, 97%, 98%, 99%, or more) sequence identity to SEQ ID NO: 7 encodes the stereocilin protein of SEQ ID NO: 5. In some embodiments, the murine stereocilin protein is encoded by a polynucleotide having the sequence of SEQ ID NO: 7.

In some embodiments, the first and second vectors are viral vectors, plasmids, cosmids, or artificial chromosomes. In some embodiments, the first and second vectors are viral vectors. In some embodiments, the viral vectors are adeno-associated virus (AAV) vectors, adenovirus vectors, or lentivirus vectors. In some embodiments, the first and second vectors are AAV vectors. In some embodiments, each of the first and second AAV vectors has an AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, or PHP.S capsid. In some embodiments, each of the first and second AAV vectors has an AAV1 capsid. In some embodiments, each of the first and second AAV vectors has an AAV9 capsid. In some embodiments, each of the first and second AAV vectors has an AAV6 capsid. In some embodiments, each of the first and second AAV vectors has an AAV8 capsid. In some embodiments, each of the first and second AAV vectors has an Anc80 capsid. In some embodiments, each of the first and second AAV vectors has an Anc80L65 capsid. In some embodiments, each of the first and second AAV vectors has a DJ/9 capsid. In some embodiments, each of the first and second AAV vectors has a 7m8 capsid. In some embodiments, each of the first and second AAV vectors has an AAV2 capsid. In some embodiments, each of the first and second AAV vectors has a PHP.B capsid. In some embodiments, each of the first and second AAV vectors has an AAV2quad(Y-F) capsid.

In another aspect, the invention provides a pharmaceutical composition containing the two-vector system of the foregoing aspect and embodiments. In some embodiments, the composition further includes a pharmaceutically acceptable carrier, diluent, or excipient.

In another aspect, the invention provides a cell (e.g., a mammalian cell, e.g., a human cell, such as an OHC, e.g., an OHC having a pathogenic mutation in the STRC gene) including the two-vector system of any of the foregoing aspects and embodiments. In some embodiments, the cell is a mammalian OHC. In some embodiments, the mammalian OHC is a human OHC.

In another aspect, the disclosure provides a method of expressing a stereocilin protein in a mammalian cell by contacting the cell with the two-vector system or the pharmaceutical composition of the foregoing aspects and embodiments. In some embodiments, the cell is a cochlear hair cell. In some embodiments, the cell is an OHC. In some embodiments, the cell is a human cell. In some embodiments, the cell is in a subject (e.g., the contacting occurs in vivo).

In another aspect, the invention provides a method of treating a subject having or at risk of developing sensorineural hearing loss by administering to an inner ear of the subject an effective amount of the two-vector system or the pharmaceutical composition of the foregoing aspects and embodiments. In some embodiments, the sensorineural hearing loss is genetic sensorineural hearing loss. In some embodiments, the genetic hearing loss is autosomal recessive hearing loss. In some embodiments of any of the foregoing aspects, the hearing loss is associated with loss of OHCs or dysfunction of OHCs. In some embodiments, the hearing loss is associated with abnormal OHC stereocilia bundle deflection or impaired connectivity between the OHC hair bundles and the tectorial membrane.

In another aspect, the invention provides a method of increasing STRC expression (e.g., wild-type STRC expression, e.g., to produce wild-type stereocilin protein) in a subject in need thereof, the method including administering to an inner ear of the subject a therapeutically effective amount of the two-vector system or the pharmaceutical composition of the foregoing aspects and embodiments.

In another aspect, the invention provides a method of preventing or reducing OHC damage or death in a subject in need thereof, including administering to an inner ear of the subject an effective amount of the two-vector system or the pharmaceutical composition of the foregoing aspects and embodiments.

In another aspect, the invention provides a method of increasing OHC survival in a subject in need thereof, including administering to an inner ear of the subject an effective amount of the two-vector system or the pharmaceutical composition of the foregoing aspects and embodiments.

In another aspect, the invention provides a method of increasing or improving OHC hair bundle attachment to the tectorial membrane in a subject in need thereof, including administering to an inner ear of the subject an effective amount of the two-vector system or the pharmaceutical composition of the foregoing aspects and embodiments.

In some embodiments of any of the foregoing aspects, the subject has a mutation in STRC. In some embodiments of any of the foregoing aspects, the subject has been identified as having a mutation in STRC. In some embodiments of any of the foregoing aspects, the method further includes identifying the subject as having a mutation in STRC prior to administering the two-vector system or pharmaceutical composition. In some embodiments of any of the foregoing aspects, the subject has deafness, autosomal recessive 16 (DFNB116). In some embodiments of any of the foregoing aspects, the subject has been identified as having DFNB16.

In some embodiments of any of the foregoing aspects, the method further includes evaluating the hearing of the subject prior to administering two-vector system or pharmaceutical composition (e.g., evaluating hearing using standard tests, such as audiometry, auditory brainstem response (ABR), electrocochleography (ECOG), or otoacoustic emissions).

In some embodiments of any of the foregoing aspects, the method further includes evaluating the hearing of the subject after administering the two-vector system or pharmaceutical composition (e.g., evaluating hearing using standard tests, such as audiometry, ABR, ECOG, or otoacoustic emissions).

In some embodiments of any of the foregoing aspects, the two-vector system or pharmaceutical composition is locally administered. In some embodiments, the two-vector system or pharmaceutical composition is administered to the ear of the subject (e.g., administered to the inner ear, e.g., into the perilymph or endolymph, such as to or through the oval window, round window, or horizontal canal, or by transtympanic or intratympanic injection). In some embodiments, the vectors in the two-vector system are administered concurrently. In some embodiments, the vectors in the two-vector system are administered sequentially.

In some embodiments of any of the foregoing aspects, the nucleic acid vector or composition is administered in an amount sufficient to prevent or reduce hearing loss, delay the development of hearing loss, slow the progression of hearing loss, improve hearing, improve speech discrimination, improve hair cell function, prevent or reduce hair cell damage, prevent or reduce hair cell death, promote or increase hair cell survival, improve OHC hair bundle attachment to the tectorial membrane, or increase STRC expression in a hair cell.

In some embodiments of any of the foregoing aspects, the subject is a human.

In another aspect, the invention provides a kit containing two-vector system or the pharmaceutical composition of the foregoing aspects and embodiments.

### Definitions

As used herein, the term “about” refers to a value that is within 10% above or below the value being described.

As used herein, “administration” refers to providing or giving a subject a therapeutic agent (e.g., a two-vector system containing an oncomodulin (OCM) promoter operably linked to a polynucleotide encoding a stereocilin protein), by any effective route. Exemplary routes of administration are described herein below.

As used herein, the phrase “administering to the inner ear” refers to providing or giving a therapeutic agent described herein to a subject by any route that allows for transduction of inner ear cells.

Exemplary routes of administration to the inner ear include administration into the perilymph or endolymph, such as to or through the oval window, round window, or semicircular canal (e.g., horizontal canal), or by transtympanic or intratympanic injection, e.g., administration to an OHC.

As used herein, the term “cell type” refers to a group of cells sharing a phenotype that is statistically separable based on gene expression data. For instance, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, the term “cochlear hair cell” refers to group of specialized cells in the inner ear that are involved in sensing sound. There are two types of cochlear hair cells: inner hair cells and outer hair cells. Damage to cochlear hair cells and genetic mutations that disrupt cochlear hair cell function are implicated in hearing loss and deafness.

As used herein, the terms “conservative mutation,” “conservative substitution,” and “conservative amino acid substitution” refer to a substitution of one or more amino acids for one or more different amino acids that exhibit similar physicochemical properties, such as polarity, electrostatic charge, and steric volume. These properties are summarized for each of the twenty naturally occurring amino acids in table 1, below.

TABLE 1
Representative physicochemical properties
of naturally occurring amino acids

| | | | | Electrostatic | |
|---|---|---|---|---|---|
| | 3 | 1 | Side- | character at | |
| | Letter | Letter | chain | physiological | Steric |
| Amino Acid | Code | Code | Polarity | pH (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral | large |
| | | | | and cationic | |
| | | | | forms in | |
| | | | | equilibrium | |
| | | | | at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | non-polar | neutral | intermediate |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include (i) G, A, V, L, and I; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

As used herein, the term “degradation signal sequence” refers to a sequence (e.g., a nucleotide sequence that can be translated into an amino acid sequence) that mediates the degradation of a polypeptide in which it is contained. Degradation signal sequences can be included in the nucleic acid vectors of the invention to reduce or prevent the expression of portions of stereocilin proteins that have not undergone recombination and/or splicing.

The terms “derived” and “derivative” as used herein refer to a nucleic acid, peptide, or protein or a variant or analog thereof comprising one or more mutations and/or chemical modifications as compared to a corresponding full-length wild-type nucleic acid, peptide, or protein. Non-limiting examples of chemical modifications involving nucleic acids include, for example, modifications to the base moiety, sugar moiety, phosphate moiety, phosphate-sugar backbone, or a combination thereof.

As used herein, the terms “effective amount,” “therapeutically effective amount,” and a “sufficient amount” of a composition, vector construct, or viral vector described herein refer to a quantity sufficient to, when administered to the subject, including a mammal, for example a human, effect beneficial or desired results, including clinical results, and, as such, an “effective amount” or synonym thereto depends upon the context in which it is being applied. For example, in the context of treating sensorineural hearing loss, it is an amount of the composition, vector construct, or viral vector sufficient to achieve a treatment response as compared to the response obtained without administration of the composition, vector construct, or viral vector. The amount of a given composition described herein that will correspond to such an amount will vary depending upon various factors, such as the given agent, the pharmaceutical formulation, the route of administration, the type of disease or disorder, the identity of the subject (e.g., age, sex, weight) or host being treated, and the like, but can nevertheless be routinely determined by one skilled in the art. Also, as used herein, a “therapeutically effective amount” of a composition, vector construct, or viral vector of the present disclosure is an amount which results in a beneficial or desired result in a subject as compared to a control. As defined herein, a therapeutically effective amount of a composition, vector construct, or viral vector of the present disclosure may be readily determined by one of ordinary skill by routine methods known in the art. Dosage regimen may be adjusted to provide the optimum therapeutic response.

As used herein, the term “endogenous” refers to a molecule (e.g., a polypeptide, nucleic acid, or cofactor) that is found naturally in a particular organism (e.g., a human) or in a particular location within an organism (e.g., an organ, a tissue, or a cell, such as a human cell, e.g., an OHC).

As used herein, the term “express” refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell, e.g., a human OHC).

Exogenous materials include those that are provided from an external source to an organism or to cultured matter extracted there from.

As used herein, the term “exon” refers to a region within the coding region of a gene, the nucleotide sequence of which determines the amino acid sequence of the corresponding protein. The term exon also refers to the corresponding region of the RNA transcribed from a gene. Exons are transcribed into pre-mRNA and may be included in the mature mRNA depending on the alternative splicing of the gene. Exons that are included in the mature mRNA following processing are translated into protein, wherein the sequence of the exon determines the amino acid composition of the protein.

As used herein, the term “heterologous” refers to a combination of elements that is not naturally occurring. For example, a heterologous transgene refers to a transgene that is not naturally expressed by the promoter to which it is operably linked.

As used herein, the terms “increasing” and “decreasing” refer to modulation resulting in, respectively, greater or lesser amounts, of function, expression, or activity of a metric relative to a reference. For example, subsequent to administration of a composition in a method described herein, the amount of a marker of a metric (e.g., transgene expression) as described herein may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the marker prior to administration. Generally, the metric is measured subsequent to administration at a time that the administration has had the recited effect, e.g., at least one week, one month, 3 months, or 6 months, after a treatment regimen has begun.

As used herein, the term “intein,” also referred to as “protein intron,” refers to a portion of a protein that is typically 100-900 amino acid residues long and that is capable of self-excision and ligation of the flanking protein fragments (“exteins”) with a peptide bond. Inteins are produced during protein splicing. The term “intein” subsumes four different classes of inteins, including maxi-intein, mini-intein, trans-splicing intein, and alanine intein. Maxi-inteins refer to N- and C-terminal splicing regions of a protein containing an endonuclease domain. Endonuclease domains, also known as “homing endonuclease genes” or “HEG” refer to a class of endonucleases encoded as stand-alone genes within introns, as protein fusions with other proteins, or as self-splicing inteins. HEGs generally hydrolyze very few and often targeted DNA regions. Once a HEG hydrolyzes a piece of DNA, the gene encoding the HEG typically incorporates itself into the cleavage site, thereby increasing its allele frequency. Mini-inteins refer to N- and C-terminal splicing domains lacking the endonuclease domain. Trans-splicing inteins refer to inteins that are split into two or more domains which are further split into N-termini and C-termini. Alanine inteins refer to inteins having a splicing junction of an alanine instead of a cysteine or serine. An intein of a precursor protein may come in two genes; in such cases, the intein is designated a split “intein.”

As used herein, the term “intron” refers to a region within the coding region of a gene, the nucleotide sequence of which is not translated into the amino acid sequence of the corresponding protein. The term intron also refers to the corresponding region of the RNA transcribed from a gene. Introns are transcribed into pre-mRNA, but are removed during processing, and are not included in the mature mRNA.

As used herein, the term “outer hair cell-specific expression” or “OHC-specific expression” refers to production of an RNA transcript or polypeptide primarily within cochlear OHCs as compared to other cell types of the cochlea (e.g., spiral ganglion neurons, glia, or other cochlear cell types). OHC-specific expression of a transgene can be confirmed by comparing transgene expression (e.g., RNA or protein expression) between various cell types of the cochlea (e.g., OHCs vs. non-OHCs) using any standard technique (e.g., quantitative RT PCR, immunohistochemistry, western blot analysis, or measurement of the fluorescence of a reporter (e.g., GFP) operably linked to a promoter). An OHC-specific promoter induces expression (e.g., RNA or protein expression) of a transgene to which it is operably linked that is at least 50% greater (e.g., 50%, 75%, 100%, 125%, 150%, 175%, 200% greater or more) in OHCs compared to at least 2 (e.g., 2, 3, 4, 5, 6, 7, 8, 9, 10, or more) of the following inner ear cell types: inner hair cells, Border cells, inner phalangeal cells, inner pillar cells, outer pillar cells, first row Deiter cells, second row Deiter cells, third row Deiter cells, Hensen's cells, Claudius cells, inner sulcus cells, outer sulcus cells, spiral prominence cells, root cells, interdental cells, basal cells of the stria vascularis, intermediate cells of the stria vascularis, marginal cells of the stria vascularis, spiral ganglion neurons, Schwann cells. An OHC-specific promoter induces expression (e.g., RNA or protein expression) of a transgene to which it is operably linked that is at least 50% greater (e.g., 50%, 75%, 100%, 125%, 150%, 175%, 200% greater or more) in OHCs of the cochlea compared to other cells of the cochlea.

As used herein, “locally” or “local administration” means administration at a particular site of the body intended for a local effect and not a systemic effect. Examples of local administration are epicutaneous, inhalational, intra-articular, intrathecal, intravaginal, intravitreal, intrauterine, intra-lesional administration, lymph node administration, intratumoral administration, administration to the inner ear, and administration to a mucous membrane of the subject, wherein the administration is intended to have a local and not a systemic effect.

As used herein, the term “operably linked” refers to a first molecule joined to a second molecule, wherein the molecules are so arranged that the first molecule affects the function of the second molecule. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell. Additionally, two portions of a transcription regulatory element are operably linked to one another if they are joined such that the transcription-activating functionality of one portion is not adversely affected by the presence of the other portion. Two transcription regulatory elements may be operably linked to one another by way of a linker polynucleotide (e.g., an intervening non-coding polynucleotide) or may be operably linked to one another with no intervening nucleotides present.

As used herein, the term “plasmid” refers to a to an extrachromosomal circular double stranded DNA molecule into which additional DNA segments may be ligated. A plasmid is a type of vector, a nucleic acid molecule capable of transporting another nucleic acid to which it has been linked. Certain plasmids are capable of autonomous replication in a host cell into which they are introduced (e.g., bacterial plasmids having a bacterial origin of replication and episomal mammalian plasmids). Other vectors (e.g., non-episomal mammalian vectors) can be integrated into the genome of a host cell upon introduction into the host cell, and thereby are replicated along with the host genome. Certain plasmids are capable of directing the expression of genes to which they are operably linked.

As used herein, the term “polynucleotide” refers to a polymer of nucleosides. Typically, a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. The term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e., the succession of letters used as abbreviations for bases) that biochemically characterizes a specific nucleic acid. A polynucleotide sequence presented herein is presented in a 5′ to 3′ direction unless otherwise indicated.

As used herein, the term “promoter” refers to a recognition site on DNA that is bound by an RNA polymerase. The polymerase drives transcription of the transgene. A representative promoter of the disclosure is the oncomodulin (OCM) promoter, such as an OCM promoter having a nucleic acid sequence of any one of SEQ ID NOs: 1-3 or a variant thereof having at least 85% (e.g., at least 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-3.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

$$100 multiplied by ( the fraction X / Y )$$

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “pharmaceutical composition” refers to a mixture containing a therapeutic agent, optionally in combination with one or more pharmaceutically acceptable excipients, diluents, and/or carriers, to be administered to a subject, such as a mammal, e.g., a human, in order to prevent, treat or control a particular disease or condition affecting or that may affect the subject.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response, and other problem complications commensurate with a reasonable benefit/risk ratio.

As used herein, the term “recombinogenic region” refers to a region of homology that mediates recombination between two different sequences.

As used herein, the term “regulatory sequence” includes promoters, enhancers, and other expression control elements (e.g., polyadenylation signals) that control the transcription or translation of the polynucleotides that encode STRC. Such regulatory sequences are described, for example, in Goeddel, Gene Expression Technology: Methods in Enzymology 185 (Academic Press, San Diego, C A, 1990); incorporated herein by reference.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) isolated from a subject.

As used herein, the terms “stereocilin” and “STRC” (also known as DFNB16) refer to a protein encoded by the STRC gene and to the gene encoding this protein, respectively. In humans, STRC is tandemly duplicated, where the second copy contains a premature stop codon in exon 20, thereby producing an STRC pseudogene. In the context of the present disclosure, STRC does not refer to the STRC pseudogene. Previous studies have identified mutations in the full-length copy of STRC in humane patients with autosomal recessive non-syndromic sensorineural hearing loss (Verpy et al., Nat. Genet. 29:345-9 (2001)). Stereocilin protein expression is limited to stereocilia in hair bundles of hair cells. Stereocilin is thought to form horizontal top connectors and tectorial membrane-attachment crowns, which are required for the normal functioning of the auditory apparatus (Avan et al., PNAS 116:25948-57 (2019); Verpy et al., J. Comp. Neurol. 519:194-210 (2011)). Mice lacking stereocilin have been shown to exhibit abnormal hair cell bundles with defective cohesion and impaired hearing (Verpy et al., Nature 456:255-8 (2008)). The present disclosure provides polynucleotides encoding the full-length stereocilin protein, which, when incorporated into the vector systems described herein, may be used as a therapeutic agent for the treatment of hearing loss (e.g., sensorineural hearing loss) in subjects in need thereof. The terms “stereocilin” and “STRC” also refer to variants of wild-type stereocilin protein and nucleic acids encoding the same, respectively, such as variant proteins having at least 85% sequence identity (e.g., 86%, 87%, 88%, 89%, 90%, 91%, 92%, 93%, 94%, 95%, 96%, 97%, 98%, 99%, or 99.9% identity, or more) to the amino acid sequence of a wild-type stereocilin protein (e.g., SEQ ID NO: 4 or 5) or polynucleotides having at least 85% sequence identity (e.g., 86%, 87%, 88%, 89%, 90%, 91%, 92%, 93%, 94%, 95%, 96%, 97%, 98%, 99%, or 99.9% identity, or more) to the nucleic acid sequence of a wild-type STRC gene (e.g., SEQ ID NO: 6 or 7), provided that the STRC analog encoded retains the therapeutic function of wild-type STRC.

As used herein, the term “transcription regulatory element” refers to a polynucleotide that controls, at least in part, the transcription of a gene of interest. Transcription regulatory elements may include promoters, enhancers, and other polynucleotides (e.g., polyadenylation signals) that control or help to control gene transcription. Examples of transcription regulatory elements are described, for example, in Lorence, Recombinant Gene Expression: Reviews and Protocols (Humana Press, New York, NY, 2012).

As used herein, the terms “subject” and “patient” refer to an animal (e.g., a mammal, such as a human). A subject to be treated according to the methods described herein may be one who has been diagnosed with hearing loss (e.g., hearing loss associated with a mutation in STRC) or one at risk of developing this condition. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

As used herein, the terms “transduction” and “transduce” refer to a method of introducing a vector construct or a part thereof into a cell. Wherein the vector construct is contained in a viral vector such as for example an AAV vector, transduction refers to viral infection of the cell and subsequent transfer and integration of the vector construct or part thereof into the cell genome.

As used herein, “treatment” and “treating” in reference to a disease or condition, refer to an approach for obtaining beneficial or desired results, e.g., clinical results. Beneficial or desired results can include, but are not limited to, alleviation or amelioration of one or more symptoms or conditions; diminishment of extent of disease or condition; stabilized (i.e., not worsening) state of disease, disorder, or condition; preventing spread of disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the term “vector” refers to a nucleic acid vector, e.g., a DNA vector, such as a plasmid, cosmid, or artificial chromosome, an RNA vector, a virus, or any other suitable replicon (e.g., viral vector). A variety of vectors have been developed for the delivery of polynucleotides encoding exogenous proteins into a prokaryotic or eukaryotic cell. Examples of such expression vectors are described in, e.g., Gellissen, Production of Recombinant Proteins: Novel Microbial and Eukaryotic Expression Systems (John Wiley & Sons, Marblehead, M A, 2006). Expression vectors suitable for use with the compositions and methods described herein contain a polynucleotide sequence as well as, e.g., additional sequence elements used for the expression of proteins and/or the integration of these polynucleotide sequences into the genome of a mammalian cell. Certain vectors that can be used for the expression of STRC as described herein include vectors that contain regulatory sequences, such as promoter and enhancer regions, which direct gene transcription. Other useful vectors for expression of STRC contain polynucleotide sequences that enhance the rate of translation of STRC or improve the stability or nuclear export of the mRNA that results from gene transcription. These sequence elements include, e.g., 5′ and 3′ untranslated regions and a polyadenylation signal site to direct efficient transcription of the gene carried on the expression vector. The expression vectors suitable for use with the compositions and methods described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

As used herein, the term “wild-type” refers to a genotype with the highest frequency for a particular gene in a given organism.

### BRIEF DESCRIPTION OF THE DRAWINGS

FIGS. 1A-1B are a series of fluorescent images of mouse cochlea transduced with either an adeno-associated virus (AAV) vector expressing green fluorescent protein (GFP) under the control of the ubiquitous cytomegalovirus (CMV) promoter (FIG. 1A), or an AAV vector expressing GFP under control of an oncomodulin (OCM) promoter (SEQ ID NO: 1; FIG. 11B). Native GFP fluorescence is shown. Using a ubiquitous promoter, AAV-CMV-GFP induced GFP expression in many cell types within the cochlea including inner hair cells (IHCs), outer hair cells (OHCs), spiral ganglion neurons, mesenchymal cells, and glia (FIG. 1A). Using an OHC-specific promoter, AAV-OCM (SEQ ID NO: 1)-GFP induced GFP expression exclusively in OHCs (FIG. 11B).

FIGS. 2A and 2B are a series of micrographs of single paraffin sections from a cochlea basal turn of two non-human primates (Macaca fascicularis) administered an AAV vector expressing H2B-GFP under control of the OCM promoter of SEQ ID NO: 1. FIG. 2A is a micrograph of single paraffin section from a first animal and FIG. 2B is a micrograph of single paraffin section from a second animal. Panel A in FIG. 2A and panel B in FIG. 2B (upper images) show a grey scale conversion of the area around the organ of Corti with Hematoxylin-stained nuclei originally in blue and H2B-GFP antibody originally stained in red. Panel A′ in FIG. 2A and panel B′ in FIG. 2B (lower images) show the remaining signal after removing the signal for blue Hematoxylin; H2B-GFP positive (red) nuclei remain visible as darker colors after greyscale conversion. The scale bars represent 100 μm. Inner hair cells (IHCs) and outer hair cells (OHCs) are highlighted for orientation.

FIGS. 3A-3C are a series of fluorescent images of stereocilin expression in the mouse organ of Corti in a 200 μm² ROI at 16 kHz. FIG. 3A shows stereocilin antibody staining at the tips of the outer hair cell (OHC) stereocilia in a wild-type CBA/CaJ mouse. As shown in FIG. 3B, 232 bp STRC knockout (KO) animals lacked the signal for the antibody. FIG. 3C shows stereocilin antibody staining in a 232 bp STRC KO mouse administered dual Anc80 vectors, in which the first vector carried a CMV promoter and nucleotides 1-3200 of the murine STRC cDNA and the vector second carried nucleotides 2201-5430, creating a 1000 bp overlap between the two cDNA in the two vectors. De-novo stereocilin protein expression could be observed at the tips of the OHC stereocilia and in the body of inner hair cells of the organ of Corti in treated 232 bp STRC KO mice.

FIGS. 4A-4C are a series of graphs showing improvement of auditory function in Anc80-CMV-mStrc treated 232 bp STRC KO mice and correlation to OHC STRC expression. Untreated contralateral ears showed near absent DPOAEs and highly elevated ABR thresholds indicative of loss of OHC function (FIGS. 4A-4B, open circles), while treated 232 bp STRC KO animals showed recovery of hearing thresholds (FIGS. 4A-4B, filled circles). The best responder of the treated animals (FIGS. 4A-4B, black squares) showed close to wild type (FIGS. 4A-4B, triangles) hearing thresholds. A high fraction of OHCs of 232 bp STRC KO mice expressing stereocilin after treatment with AAV-Anc80-CMV-mStrc was found to promote hearing recovery (FIG. 4C).

FIGS. 5A-5B are an image and a graph showing that transfection of HEK293T cells with a two-vector split intein system led to reconstitution of full-length stereocilin. FIG. 5A is a representative image of a Western blot against stereocilin protein and beta-actin. FIG. 5B is a densitometry quantification of full-length stereocilin band intensity relative to actin and indicates the relative expression of full-length stereocilin protein for the negative (GFP) control, positive full-length control, and the Npu intein construct.

FIGS. 6A-6B are maps of plasmids P959 and P724, respectively, used to create an overlapping dual vector system for expressing stereocilin (STRC) under the control of a murine OCM promoter.

FIGS. 7A-7B are maps of plasmids P960 and P726, respectively, used to create a dual hybrid vector system for expressing STRC under the control of a murine OCM promoter.

### DETAILED DESCRIPTION

Described herein are compositions and methods for the treatment of sensorineural hearing loss in a subject (such as a mammalian subject, for instance, a human) by administering a first nucleic acid vector containing a promoter, such as an oncomodulin (OCM) promoter, and a polynucleotide encoding an N-terminal portion of a stereocilin (STRC) protein (e.g., wild-type (WT) STRC protein) and a second nucleic acid vector containing a polynucleotide encoding a C-terminal portion of a STRC protein and a polyadenylation (poly(A)) sequence. When introduced into a mammalian cell, such as a cochlear outer hair cell (OHC), the polynucleotides encoded by the two nucleic acid vectors can combine to form a polynucleotide that encodes the full-length STRC protein. The disclosure also features two-vector expression systems (e.g., overlapping dual vectors, trans-splicing vectors, dual hybrid vectors, and split intein trans-splicing vectors) containing the aforementioned polynucleotides. The compositions and methods described herein can be used to express polynucleotides encoding STRC specifically in OHCs, and, therefore, the compositions described herein can be administered to a subject (such as a mammalian subject, for instance, a human) to treat disorders caused by dysfunction of OHCs, such as hearing loss (e.g., sensorineural hearing loss) and auditory neuropathy.

### Stereocilin

Stereocilin (also known as DFNB16) is a protein encoded by the STRC gene on chromosome 15q15, which contains 29 exons spanning approximately 19 kb of the genome. The STRC gene is tandemly duplicated, where the second copy contains a premature stop codon in exon 20, thereby producing an STRC pseudogene. Previous studies have identified two frameshift mutations and a large deletion in the full-length copy of STRC in two families with autosomal recessive non-syndromic sensorineural hearing loss (Verpy et al., Nat. Genet. 29:345-9 (2001)). Stereocilin protein expression is limited to stereocilia in hair bundles of inner ear hair cells and is thought to form horizontal top connectors and tectorial membrane-attachment crowns, which are required for the normal functioning of the auditory apparatus (Avan et al., PNAS 116:25948-57 (2019); Verpy et al., J. Comp. Neurol. 519:194-210 (2011)). Mice lacking stereocilin have been shown to exhibit abnormal hair cell bundles with defective cohesion and impaired hearing (Verpy et al., Nature 456:255-8 (2008)).

The compositions and methods described herein can be used to treat sensorineural hearing loss by administering a first nucleic acid vector containing a polynucleotide encoding an N-terminal portion of a stereocilin protein and a second nucleic acid vector containing a polynucleotide encoding a C-terminal portion of a stereocilin protein. The full-length STRC coding sequence is too large to include in the type of vector that is commonly used for gene therapy (e.g., an adeno-associated virus (AAV) vector, which is thought to have a packaging limit of 5 kb). The compositions and methods described herein overcome this problem by dividing the STRC coding sequence between two different nucleic acid vectors such that the full-length STRC sequence can be reconstituted in a cell. These compositions and methods can be used to treat subjects having one or more mutations in the STRC gene, e.g., an STRC mutation that reduces STRC expression, reduces STRC function, or is associated with hearing loss (e.g., a subject having DFNB16). When the first and second nucleic acid vectors are administered in a composition, the polynucleotides encoding the N-terminal and C-terminal portions of stereocilin can combine within a cell (e.g., a human cell, e.g., a cochlear hair cell) to form a single polynucleotide that contains the full-length STRC coding sequence (e.g., through homologous recombination and/or splicing).

The nucleic acid vectors used in the compositions and methods described herein include polynucleotide sequences that encode wild-type stereocilin, or a variant thereof, such as polynucleotide sequences that, when combined, encode a protein having at least 85% sequence identity (e.g., 85%, 90%, 95%, 96%, 97%, 98%, 99%, or more, sequence identity) to the amino acid sequence of wild-type mammalian (e.g., human or mouse) stereocilin. The polynucleotides used in the nucleic acid vectors described herein encode an N-terminal portion and a C-terminal portion of a stereocilin amino acid sequence in Table 2 below (e.g., two portions that, when combined, encode a full-length stereocilin amino acid sequence listed in Table 2, e.g., SEQ ID NO: 4 or SEQ ID NO: 5).

According to the methods described herein, a subject can be administered a composition containing a first nucleic acid vector and a second nucleic acid vector that contain an N-terminal and C-terminal portion, respectively, of a polynucleotide sequence encoding the amino acid sequence of SEQ ID NO: 4 or SEQ ID NO: 5, or a polynucleotide sequence encoding an amino acid sequence having at least 85% sequence identity (e.g., 85%, 86%, 87%, 88%, 89%, 90%, 91%, 92%, 93%, 94%, 95%, 96%, 97%, 98%, 99%, or more, sequence identity) to the amino acid sequence of SEQ ID NO: 4 or SEQ ID NO: 5, or a polynucleotide sequence encoding an amino acid sequence that contains one or more conservative amino acid substitutions relative to SEQ ID NO: 4 or SEQ ID NO: 5 (e.g., 1, 2, 3, 4, 5, 6, 7, 8, 9, or 10 or more conservative amino acid substitutions), provided that the stereocilin analog encoded retains the therapeutic function of wild-type STRC. In some embodiments, no more than 10% of the amino acids in the N-terminal portion of the stereocilin protein and no more than 10% of the amino acids in the C-terminal portion of the stereocilin protein may be replaced with conservative amino acid substitutions. The stereocilin protein may be encoded by a polynucleotide having the sequence of SEQ ID NO: 5 or SEQ ID NO: 6. The stereocilin protein may also be encoded by a polynucleotide having single nucleotide variants (SNVs) that have been found to be non-pathogenic in human subjects. The stereocilin protein may be a human stereocilin protein or may be a homolog of the human stereocilin protein from another mammalian species (e.g., mouse, rat, cow, horse, goat, sheep, donkey, cat, dog, rabbit, guinea pig, or other mammal).

TABLE 2
STRC Sequences

| SEQ ID | | |
|---|---|---|
| NO. | Sequence Name | Sequence |
| 4 | Wild-type human | MALSLWPLLLLLLLLLLLSFAVTLAPTGPHSLDPGLSFLKSLLSTLDQ |
| | stereocilin protein, | APQGSLSRSRFFTFLANISSSFEPGRMGEGPVGEPPPLQPPALRLH |
| | UniProt ID: | DFLVTLRGSPDWEPMLGLLGDMLALLGQEQTPRDFLVHQAGVLGG |
| | Q7RTU9 | LVEVLLGALVPGGPPTPTRPPCTRDGPSDCVLAADWLPSLLLLLEG |
| | | TRWQALVQVQPSVDPTNATGLDGREAAPHFLQGLLGLLTPTGELG |
| | | SKEALWGGLLRTVGAPLYAAFQEGLLRVTHSLQDEVFSILGQPEPD |
| | | TNGQCQGGNLQQLLLWGVRHNLSWDVQALGFLSGSPPPPPALLH |
| | | CLSTGVPLPRASQPSAHISPRQRRAITVEALCENHLGPAPPYSISNF |
| | | SIHLLCQHTKPATPQPHPSTTAICQTAVWYAVSWAPGAQGWLQAC |
| | | HDQFPDEFLDAICSNLSFSALSGSNRRLVKRLCAGLLPPPTSCPEG |
| | | LPPVPLTPDIFWGCFLENETLWAERLCGEASLQAVPPSNQAWVQH |
| | | VCQGPTPDVTASPPCHIGPCGERCPDGGSFLVMVCANDTMYEVLV |
| | | PFWPWLAGQCRISRGGNDTCFLEGLLGPLLPSLPPLGPSPLCLTPG |
| | | PFLLGMLSQLPRCQSSVPALAHPTRLHYLLRLLTFLLGPGAGGAEA |
| | | QGMLGRALLLSSLPDNCSFWDAFRPEGRRSVLRTIGEYLEQDEEQ |
| | | PTPSGFEPTVNPSSGISKMELLACFSPVLWDLLQREKSVWALQILV |
| | | QAYLHMPPENLQQLVLSAEREAAQGFLTLMLQGKLQGKLQVPPSE |
| | | EQALGRLTALLLQRYPRLTSQLFIDLSPLIPFLAVSDLMRFPPSLLAN |
| | | DSVLAAIRDYSPGMRPEQKEALAKRLLAPELFGEVPAWPQELLWA |
| | | VLPLLPHLPLENFLQLSPHQIQALEDSWPAAGLGPGHARHVLRSLV |
| | | NQSVQDGEEQVRRLGPLACFLSPEELQSLVPLSDPTGPVERGLLE |
| | | CAANGTLSPEGRVAYELLGVLRSSGGAVLSPRELRVWAPLFSQLG |
| | | LRFLQELSEPQLRAMLPVLQGTSVTPAQAVLLLGRLLPRHDLSLEEL |
| | | CSLHLLLPGLSPQTLQAIPRRVLVGACSCLAPELSRLSACQTAALLQ |
| | | TFRVKDGVKNMGTTGAGPAVCIPGQPIPTTWPDCLLPLLPLKLLQL |
| | | DSLALLANRRRYWELPWSEQQAQFLWKKMQVPTNLTLRNLQALG |
| | | TLAGGMSCEFLQQINSMVDFLEVVHMIYQLPTRVRGSLRACIWAEL |
| | | QRRMAMPEPEWTTVGPELNGLDSKLLLDLPIQLMDRLSNESIMLVV |
| | | ELVQRAPEQLLALTPLHQAALAERALQNLAPKETPVSGEVLETLGP |
| | | LVGFLGTESTRQIPLQILLSHLSQLQGFCLGETFATELGWLLLQESV |
| | | LGKPELWSQDEVEQAGRLVFTLSTEAISLIPREALGPETLERLLEKQ |
| | | QSWEQSRVGQLCREPQLAAKKAALVAGVVRPAAEDLPEPVPNCA |
| | | DVRGTFPAAWSATQIAEMELSDFEDCLTLFAGDPGLGPEELRAAM |
| | | GKAKQLWGPPRGFRPEQILQLGRLLIGLGDRELQELILVDWGVLST |
| | | LGQIDGWSTTQLRIVVSSFLRQSGRHVSHLDFVHLTALGYTLCGLR |
| | | PEELQHISSWEFSQAALFLGTLHLQCSEEQLEVLAHLLVLPGGFGPI |
| | | SNWGPEIFTEIGTIAAGIPDLALSALLRGQIQGVTPLAISVIPPPKFAV |
| | | VFSPIQLSSLTSAQAVAVTPEQMAFLSPEQRRAVAWAQHEGKESP |
| | | EQQGRSTAWGLQDWSRPSWSLVLTISFLGHLL |
| 5 | Murine stereocilin | MALSLQPQLLLLLSLLPQEVTSAPTGPQSLDAGLSLLKSFVATLDQA |
| | protein | PQRSLSQSRFSAFLANISSSFQLGRMGEGPVGEPPPLQPPALRLH |
| | (NP_536707.2) | DFLVTLRGSPDWEPMLGLLGDVLALLGQEQTPRDFLVHQAGVLGG |
| | | LVEALLGALVPGGPPAPTRPPCTRDGPSDCVLAADWLPSLMLLLEG |
| | | TRWQALVQLQPSVDPTNATGLDGREPAPHFLQGLLGLLTPAGELG |
| | | SEEALWGGLLRTVGAPLYAAFQEGLLRVTHSLQDEVFSIMGQPEP |
| | | DASGQCQGGNLQQLLLWGMRNNLSWDARALGFLSGSPPPPPALL |
| | | HCLSRGVPLPRASQPAAHISPRQRRAISVEALCENHSGPEPPYSIS |
| | | NFSIYLLCQHIKPATPRPPPTTPRPPPTTPQPPPTTTQPIPDTTQPPP |
| | | VTPRPPPTTPQPPPSTAVICQTAVWYAVSWAPGARGWLQACHDQ |
| | | FPDQFLDMICGNLSFSALSGPSRPLVKQLCAGLLPPPTSCPPGLIPV |
| | | PLTPEIFWGCFLENETLWAERLCVEDSLQAVPPRNQAWVQHVCRG |
| | | PTLDATDFPPCRVGPCGERCPDGGSFLLMVCANDTLYEALVPFWA |
| | | WLAGQCRISRGGNDTCFLEGMLGPLLPSLPPLGPSPLCLAPGPFLL |
| | | GMLSQLPRCQSSVPALAHPTRLHYLLRLLTFLLGPGTGGAETQGML |
| | | GQALLLSSLPDNCSFWDAFRPEGRRSVLRTVGEYLQREEPTPPGL |
| | | DSSLSLGSGMSKMELLSCFSPVLWDLLQREKSVWALRTLVKAYLR |
| | | MPPEDLQQLVLSAEMEAAQGFLTLMLRSWAKLKVQPSEEQAMGR |
| | | LTALLLQRYPRLTSQLFIDMSPLIPFLAVPDLMRFPPSLLANDSVLAAI |
| | | RDHSSGMKPEQKEALAKRLLAPELFGEVPDWPQELLWAALPLLPH |
| | | LPLESFLQLSPHQIQALEDSWPVADLGPGHARHVLRSLVNQSMED |
| | | GEEQVLRLGSLACFLSPEELQSLVPLSDPMGPVEQGLLECAANGTL |
| | | SPEGRVAYELLGVLRSSGGTVLSPRELRVWAPLFPQLGLRFLQELS |
| | | ETQLRAMLPALQGASVTPAQAVLLFGRLLPKHDLSLEELCSLHPLLP |
| | | GLSPQTLQAIPKRVLVGACSCLGPELSRLSACQIAALLQTFRVKDGV |
| | | KNMGAAGAGSAVCIPGQPTTWPDCLLPLLPLKLLQLDAAALLANRR |
| | | LYRQLPWSEQQAQFLWKKMQVPTNLSLRNLQALGNLAGGMTCEF |
| | | LQQISSMVDFLDVVHMLYQLPTGVRESLRACIWTELQRRMTMPEP |
| | | ELTTLGPELSELDTKLLLDLPIQLMDRLSNDSIMLVVEMVQGAPEQL |
| | | LALTPLHQTALAERALKNLAPKETPISKEVLETLGPLVGFLGIESTRRI |
| | | PLPILLSHLSQLQGFCLGETFATELGWLLLQEPVLGKPELWSQDEIE |
| | | QAGRLVFTLSAEAISSIPREALGPETLERLLGKHQSWEQSRVGHLC |
| | | GESQLAHKKAALVAGIVHPAAEGLQEPVPNCADIRGTFPAAWSATQ |
| | | ISEMELSDFEDCLSLFAGDPGLGPEELRAAMGKAKQLWGPPRGFR |
| | | PEQILQLGRLLIGLGERELQELTLVDWGVLSSLGQIDGWSSMQLRA |
| | | VVSSFLRQSGRHVSHLDFIYLTALGYTVCGLRPEELQHISSWEFSQ |
| | | AALFLGSLHLPCSEEQLEVLAYLLVLPGGFGPVSNWGPEIFTEIGTIA |
| | | AGIPDLALSALLRGQIQGLTPLAISVIPAPKFAVVENPIQLSSLTRGQA |
| | | VAVTPEQLAYLSPEQRRAVAWAQHEGKEIPEQLGRNSAWGLYDW |
| | | FQASWALALPVSIFGHLL |
| 6 | Polynucleotide | ATGGCTCTCAGCCTCTGGCCCCTGCTGCTGCTGCTGCTGCTGC |
| | encoding full-length | TGCTGCTGCTGTCCTTTGCAGTGACTCTGGCCCCTACTGGGCCT |
| | WT human | CATTCCCTGGACCCTGGTCTCTCCTTCCTGAAGTCATTGCTCTC |
| | stereocilin (from | CACTCTGGACCAGGCTCCCCAGGGCTCCCTGAGCCGCTCACGG |
| | NM_153700.2), | TTCTTTACATTCCTGGCCAACATTTCTTCTTCCTTTGAGCCTGGG |
| | encodes the protein | AGAATGGGGGAAGGACCAGTAGGAGAGCCCCCACCTCTCCAGC |
| | of SEQ ID NO: 4 | CGCCTGCTCTGCGGCTCCATGATTTTCTAGTGACACTGAGAGGT |
| | (includes stop | AGCCCCGACTGGGAGCCAATGCTAGGGCTGCTAGGGGATATGC |
| | codon) | TGGCACTGCTGGGACAGGAGCAGACTCCCCGAGATTTCCTGGT |
| | | GCACCAGGCAGGGGTGCTGGGTGGACTTGTGGAGGTGCTGCT |
| | | GGGAGCCTTAGTTCCTGGGGGCCCCCCTACCCCAACTCGGCCC |
| | | CCATGCACCCGTGATGGGCCGTCTGACTGTGTCCTGGCTGCTG |
| | | ACTGGTTGCCTTCTCTGCTGCTGTTGTTAGAGGGCACACGCTGG |
| | | CAAGCTCTGGTGCAGGTGCAGCCCAGTGTGGACCCCACCAATG |
| | | CCACAGGCCTCGATGGGAGGGAGGCAGCTCCTCACTTTTTGCA |
| | | GGGTCTGTTGGGTTTGCTTACCCCAACAGGGGAGCTAGGCTCC |
| | | AAGGAGGCTCTTTGGGGCGGTCTGCTACGCACAGTGGGGGCCC |
| | | CCCTCTATGCTGCCTTTCAGGAGGGGCTGCTCCGTGTCACTCAC |
| | | TCCCTGCAGGATGAGGTCTTCTCCATTTTGGGGCAGCCAGAGC |
| | | CTGATACCAATGGGCAGTGCCAGGGAGGTAACCTTCAACAGCT |
| | | GCTCTTATGGGGCGTCCGGCACAACCTTTCCTGGGATGTCCAG |
| | | GCGCTGGGCTTTCTGTCTGGATCACCACCCCCACCCCCTGCCC |
| | | TCCTTCACTGCCTGAGCACGGGCGTGCCTCTGCCCAGAGCTTC |
| | | TCAGCCGTCAGCCCACATCAGCCCACGCCAACGGCGAGCCATC |
| | | ACTGTGGAGGCCCTCTGTGAGAACCACTTAGGCCCAGCACCAC |
| | | CCTACAGCATTTCCAACTTCTCCATCCACTTGCTCTGCCAGCACA |
| | | CCAAGCCTGCCACTCCACAGCCCCATCCCAGCACCACTGCCAT |
| | | CTGCCAGACAGCTGTGTGGTATGCAGTGTCCTGGGCACCAGGT |
| | | GCCCAAGGCTGGCTACAGGCCTGCCACGACCAGTTTCCTGATG |
| | | AGTTTTTGGATGCGATCTGCAGTAACCTCTCCTTTTCAGCCCTGT |
| | | CTGGCTCCAACCGCCGCCTGGTGAAGCGGCTCTGTGCTGGCCT |
| | | GCTCCCACCCCCTACCAGCTGCCCTGAAGGCCTGCCCCCTGTT |
| | | CCCCTCACCCCAGACATCTTTTGGGGCTGCTTCTTGGAGAATGA |
| | | GACTCTGTGGGCTGAGCGACTGTGTGGGGAGGCAAGTCTACAG |
| | | GCTGTGCCCCCCAGCAACCAGGCTTGGGTCCAGCATGTGTGCC |
| | | AGGGCCCCACCCCAGATGTCACTGCCTCCCCACCATGCCACAT |
| | | TGGACCCTGTGGGGAACGCTGCCQGGATGGGGGCAGCTTCCT |
| | | GGTGATGGTCTGTGCCAATGACACCATGTATGAGGTCCTGGTGC |
| | | CCTTCTGGCCTTGGCTAGCAGGCCAATGCAGGATAAGTCGTGG |
| | | GGGCAATGACACTTGCTTCCTAGAAGGGCTGCTGGGCCCCCTT |
| | | CTGCCCTCTCTGCCACCACTGGGACCATCCCCACTCTGTCTGAC |
| | | CCCTGGCCCCTTCCTCCTTGGCATGCTATCCCAGTTGCCACGCT |
| | | GTCAGTCCTCTGTCCCAGCTCTTGCTCACCCCACACGCCTACAC |
| | | TATCTCCTCCGCCTGCTGACCTTCCTCTTGGGTCCAGGGGCTGG |
| | | GGGCGCTGAGGCCCAGGGGATGCTGGGTCGGGCCCTACTGCT |
| | | CTCCAGTCTCCCAGACAACTGCTCCTTCTGGGATGCCTTTCGCC |
| | | CAGAGGGCCGGCGCAGTGTGCTACGGACGATTGGGGAATACCT |
| | | GGAACAAGATGAGGAGCAGCCAACCCCATCAGGCTTTGAACCC |
| | | ACTGTCAACCCCAGCTCTGGTATAAGCAAGATGGAGCTGCTGGC |
| | | CTGCTTTAGTCCTGTGCTGTGGGATCTGCTCCAGAGGGAAAAGA |
| | | GTGTTTGGGCCCTGCAGATTCTAGTGCAGGCGTACCTGCATATG |
| | | CCCCCAGAAAACCTCCAGCAGCTGGTGCTTTCAGCAGAGAGGG |
| | | AGGCTGCACAGGGCTTCCTGACACTCATGCTGCAGGGGAAGCT |
| | | GCAGGGGAAGCTGCAGGTACCACCATCCGAGGAGCAGGCCCT |
| | | GGGTCGCCTGACAGCCCTGCTGCTCCAGCGGTACCCACGCCTC |
| | | ACCTCCCAGCTCTTCATTGACCTGTCACCACTCATCCCTTTCTTG |
| | | GCTGTCTCTGACCTGATGCGCTTCCCACCATCCCTGTTAGCCAA |
| | | CGACAGTGTCCTGGCTGCCATCCGGGATTACAGCCCAGGAATG |
| | | AGGCCTGAACAGAAGGAGGCTCTGGCAAAGCGACTGCTGGCCC |
| | | CTGAACTGTTTGGGGAAGTGCCTGCCTGGCCCCAGGAGCTGCT |
| | | GTGGGCAGTGCTGCCCCTGCTCCCCCACCTCCCTCTGGAGAAC |
| | | TTTTTGCAGCTCAGCCCTCACCAGATCCAGGCCCTGGAGGATAG |
| | | CTGGCCAGCAGCAGGTCTGGGGCCAGGGCATGCCCGCCATGT |
| | | GCTGCGCAGCCTGGTAAACCAGAGTGTCCAGGATGGTGAGGAG |
| | | CAGGTACGCAGGCTTGGGCCCCTCGCCTGTTTCCTGAGCCCTG |
| | | AGGAGCTGCAGAGCCTAGTGCCCCTGAGTGATCCAACGGGGCC |
| | | AGTAGAACGGGGGCTGCTGGAATGTGCAGCCAATGGGACCCTC |
| | | AGCCCAGAAGGACGGGTGGCATATGAACTTCTGGGTGTGTTGC |
| | | GCTCATCTGGAGGAGCGGTGCTGAGCCCCCGGGAGCTGCGGG |
| | | TCTGGGCCCCTCTCTTCTCTCAGCTGGGCCTCCGCTTCCTTCAG |
| | | GAGCTGTCAGAGCCCCAGCTTAGAGCCATGCTTCCTGTCCTGCA |
| | | GGGAACTAGTGTTACACCTGCTCAGGCTGTCCTGCTGCTTGGAC |
| | | GGCTCCTTCCTAGGCACGATCTATCCCTGGAGGAACTCTGCTCC |
| | | TTGCACCTTCTGCTACCAGGCCTCAGCCCCCAGACACTCCAGG |
| | | CCATCCCTAGGCGAGTCCTGGTCGGGGCTTGTTCCTGCCTGGC |
| | | CCCTGAACTGTCACGCCTCTCAGCCTGCCAGACCGCAGCACTG |
| | | CTGCAGACCTTTCGGGTTAAAGATGGTGTTAAAAATATGGGTAC |
| | | AACAGGTGCTGGTCCAGCTGTGTGTATCCCTGGTCAGCCTATTC |
| | | CCACCACCTGGCCAGACTGCCTGCTTCCCCTGCTCCCATTAAAG |
| | | CTGCTACAACTGGATTCCTTGGCTCTTCTGGCAAATCGAAGACG |
| | | CTACTGGGAGCTGCCCTGGTCTGAGCAGCAGGCACAGTTTCTC |
| | | TGGAAGAAGATGCAAGTACCCACCAACCTTACCCTCAGGAATCT |
| | | GCAGGCTCTGGGCACCCTGGCAGGAGGCATGTCCTGTGAGTTT |
| | | CTGCAGCAGATCAACTCCATGGTAGACTTCCTTGAAGTGGTGCA |
| | | CATGATCTATCAGCTGCCCACTAGAGTTCGAGGGAGCCTGAGG |
| | | GCCTGTATCTGGGCAGAGCTACAGCGGAGGATGGCAATGCCAG |
| | | AACCAGAATGGACAACTGTAGGGCCAGAACTGAACGGGCTGGA |
| | | TAGCAAGCTACTCCTGGACTTACCGATCCAGTTGATGGACAGAC |
| | | TATCCAATGAATCCATTATGTTGGTGGTGGAGCTGGTGCAAAGA |
| | | GCTCCAGAGCAGCTGCTGGCACTGACCCCCCTCCACCAGGCAG |
| | | CCCTGGCAGAGAGGGCACTACAAAACCTGGCTCCAAAGGAGAC |
| | | TCCAGTCTCAGGGGAAGTGCTGGAGACCTTAGGCCCTTTGGTT |
| | | GGATTCCTGGGGACAGAGAGCACACGACAGATCCCCCTACAGA |
| | | TCCTGCTGTCCCATCTCAGTCAGCTGCAAGGCTTCTGCCTAGGA |
| | | GAGACATTTGCCACAGAGCTGGGATGGCTGCTATTGCAGGAGT |
| | | CTGTTCTTGGGAAACCAGAGTTGTGGAGCCAGGATGAAGTAGA |
| | | GCAAGCTGGACGCCTAGTATTCACTCTGTCTACTGAGGCAATTT |
| | | CCTTGATCCCCAGGGAGGCCTTGGGTCCAGAGACCCTGGAGCG |
| | | GCTTCTAGAAAAGCAGCAGAGCTGGGAGCAGAGCAGAGTTGGA |
| | | CAGCTGTGTAGGGAGCCACAGCTTGCTGCCAAGAAAGCAGCCC |
| | | TGGTAGCAGGGGTGGTGCGACCAGCTGCTGAGGATCTTCCAGA |
| | | ACCTGTGCCAAATTGTGCAGATGTACGAGGGACATTCCCAGCAG |
| | | CCTGGTCTGCAACCCAGATTGCAGAGATGGAGCTCTCAGACTTT |
| | | GAGGACTGCCTGACATTATTTGCAGGAGACCCAGGACTTGGGC |
| | | CTGAGGAACTGCGGGCAGCCATGGGCAAAGCAAAACAGTTGTG |
| | | GGGTCCCCCCCGGGGATTTCGTCCTGAGCAGATCCTGCAGCTT |
| | | GGTAGGCTCTTAATAGGTCTAGGAGATCGGGAACTACAGGAGCT |
| | | GATCCTAGTGGACTGGGGAGTGCTGAGCACCCTGGGGCAGATA |
| | | GATGGCTGGAGCACCACTCAGCTCCGCATTGTGGTCTCCAGTTT |
| | | CCTACGGCAGAGTGGTCGGCATGTGAGCCACCTGGACTTCGTT |
| | | CATCTGACAGCGCTGGGTTATACTCTCTGTGGACTGCGGCCAGA |
| | | GGAGCTCCAGCACATCAGCAGTTGGGAGTTCAGCCAAGCAGCT |
| | | CTCTTCCTCGGCACCCTGCATCTCCAGTGCTCTGAGGAACAACT |
| | | GGAGGTTCTGGCCCACCTACTTGTACTGCCTGGTGGGTTTGGC |
| | | CCAATCAGTAACTGGGGGCCTGAGATCTTCACTGAAATTGGCAC |
| | | CATAGCAGCTGGGATCCCAGACCTGGCTCTTTCAGCACTGCTGC |
| | | GGGGACAGATCCAGGGCGTTACTCCTCTTGCCATTTCTGTCATC |
| | | CCTCCTCCTAAATTTGCTGTGGTGTTTAGTCCCATCCAACTATCT |
| | | AGTCTCACCAGTGCTCAGGCTGTGGCTGTCACTCCTGAGCAAAT |
| | | GGCCTTTCTGAGTCCTGAGCAGCGACGAGCAGTTGCATGGGCC |
| | | CAACATGAGGGAAAGGAGAGCCCAGAACAGCAAGGTCGAAGTA |
| | | CAGCCTGGGGCCTCCAGGACTGGTCACGACCTTCCTGGTCCCT |
| | | GGTATTGACTATCAGCTTCCTTGGCCACCTGCTATGA |
| 7 | Polynucleotide | ATGGCTCTGAGCCTCCAGCCCCAGCTGCTCCTTCTCCTGTCGCT |
| | encoding full- | CCTGCCGCAGGAAGTGACTTCAGCCCCTACTGGGCCTCAGTCT |
| | length, murine wild- | TTGGATGCTGGTCTCTCCCTTCTGAAGTCATTCGTAGCCACTCT |
| | type stereocilin | GGACCAAGCTCCTCAGCGTTCCCTCAGCCAGTCACGGTTCTCTG |
| | (from | CGTTCCTGGCCAACATTTCTTCATCCTTCCAGCTTGGGAGGATG |
| | NM_080459.2), | GGGGAGGGACCGGTGGGAGAGCCCCCACCTCTCCAGCCCCCT |
| | encodes the protein | GCACTTCGACTTCATGATTTCCTCGTGACACTGAGAGGTAGCCC |
| | of SEQ ID NO: 5 | AGACTGGGAGCCAATGCTAGGGCTTCTGGGAGATGTGCTGGCA |
| | (includes stop | CTCCTGGGACAGGAACAGACTCCCCGGGACTTTTTGGTGCACC |
| | codon) | AGGCAGGTGTACTGGGTGGACTTGTAGAGGCATTGTTGGGAGC |
| | | GTTAGTTCCTGGAGGCCCCCCTGCCCCCACTCGACCCCCATGC |
| | | ACCCGTGATGGCCCTTCTGACTGTGTCCTGGCTGCTGATTGGTT |
| | | GCCTTCTCTGATGTTGTTATTAGAGGGTACACGCTGGCAGGCCC |
| | | TGGTGCAGTTGCAGCCCAGTGTGGACCCAACCAATGCCACAGG |
| | | TCTTGATGGTAGAGAGCCAGCTCCTCACTTTTTACAGGGTCTGC |
| | | TGGGCTTGCTTACCCCAGCAGGAGAGTTGGGCTCTGAGGAGGC |
| | | TCTTTGGGGTGGTCTGCTGCGCACAGTGGGGGCCCCCCTCTAT |
| | | GCTGCCTTCCAGGAGGGGCTACTGCGAGTCACTCATTCTCTGCA |
| | | AGATGAGGTCTTTTCTATTATGGGACAGCCAGAGCCTGATGCCA |
| | | GTGGGCAGTGCCAGGGAGGCAACCTTCAACAGCTGCTTTTATG |
| | | GGGCATGCGGAACAACCTTTCTTGGGACGCCCGAGCACTGGGT |
| | | TTTCTATCTGGATCACCACCTCCACCCCCTGCTCTCCTGCACTG |
| | | CCTGAGCAGAGGTGTGCCTCTGCCCAGGGCTTCCCAGCCTGCG |
| | | GCTCACATCAGCCCTCGACAGCGGCGAGCCATCTCTGTGGAGG |
| | | CCCTCTGCGAGAACCACTCAGGCCCAGAGCCACCCTACAGCAT |
| | | CTCCAACTTCTCCATCTACTTGCTCTGCCAGCACATCAAGCCTG |
| | | CCACCCCGCGGCCCCCTCCTACCACCCCACGGCCTCCTCCTAC |
| | | CACCCCACAGCCCCCTCCTACCACTACACAGCCCATTCCTGACA |
| | | CTACACAGCCCCCTCCTGTCACCCCAAGGCCTCCTCCTACCACC |
| | | CCACAACCCCCTCCTAGCACAGCTGTCATCTGCCAGACAGCTGT |
| | | ATGGTACGCAGTCTCGTGGGCACCAGGTGCCCGAGGTTGGCTC |
| | | CAAGCCTGCCATGATCAGTTTCCTGATCAATTTCTGGATATGATC |
| | | TGCGGCAACCTCTCATTTTCAGCCCTGTCTGGCCCCAGTCGTCC |
| | | TTTGGTAAAGCAGCTCTGTGCTGGCTTGCTCCCACCCCCCACTA |
| | | GCTGTCCACCAGGCCTGATCCCTGTGCCCCTCACCCCAGAAATA |
| | | TTCTGGGGCTGTTTCCTGGAGAATGAGACACTGTGGGCTGAAC |
| | | GGTTGTGTGTGGAGGACAGTCTGCAGGCTGTGCCCCCGAGGAA |
| | | CCAGGCTTGGGTTCAGCATGTGTGTCGGGGCCCCACCTTGGAC |
| | | GCCACTGATTTTCCACCGTGCCGCGTTGGACCCTGTGGGGAAC |
| | | GCTGCCCAGATGGGGGCAGCTTCCTGCTCATGGTCTGTGCCAA |
| | | TGACACTCTGTATGAAGCCTTGGTTCCCTTCTGGGCTTGGCTAG |
| | | CAGGCCAATGCAGAATTAGTCGTGGAGGAAATGATACTTGCTTT |
| | | CTAGAAGGCATGCTGGGCCCCTTGTTGCCCTCTCTGCCCCCTCT |
| | | GGGACCATCCCCACTCTGTCTGGCTCCTGGTCCTTTTCTGCTTG |
| | | GCATGTTATCCCAGTTGCCACGCTGTCAGTCCTCCGTGCCAGCC |
| | | CTCGCCCACCCCACGCGCCTACATTACCTCCTGCGCCTACTGAC |
| | | CTTCCTTCTGGGTCCAGGGACTGGGGGTGCCGAGACGCAGGG |
| | | GATGTTAGGTCAAGCCCTGCTGCTCTCTAGTCTCCCAGACAACT |
| | | GTTCATTCTGGGATGCCTTCCGCCCAGAGGGCCGGAGAAGTGT |
| | | ACTGAGGACAGTCGGAGAGTACTTGCAGCGGGAAGAGCCAACC |
| | | CCACCAGGCTTAGACTCCTCCCTCAGCCTCGGCTCTGGTATGAG |
| | | CAAGATGGAGCTTCTGTCCTGCTTCAGTCCTGTACTGTGGGATC |
| | | TACTCCAGAGAGAGAAGAGCGTTTGGGCCCTGAGGACCCTGGT |
| | | GAAGGCCTACCTGCGCATGCCTCCAGAAGACCTTCAGCAGCTT |
| | | GTGCTTTCAGCAGAGATGGAGGCTGCACAGGGCTTCCTGACGC |
| | | TCATGCTTCGTTCCTGGGCTAAGCTGAAGGTTCAACCATCCGAG |
| | | GAGCAGGCCATGGGCCGCCTGACAGCCTTGCTGCTCCAGCGGT |
| | | ACCCACGCCTCACCTCCCAACTCTTTATCGACATGTCACCGCTC |
| | | ATCCCCTTCCTGGCTGTCCCTGACCTCATGCGCTTCCCACCGTC |
| | | CCTTTTGGCCAACGACAGTGTCCTGGCTGCCATCAGGGATCACA |
| | | GCTCAGGAATGAAGCCTGAACAGAAGGAGGCCCTGGCAAAACG |
| | | ACTGCTGGCCCCTGAGCTGTTTGGAGAAGTGCCTGATTGGCCC |
| | | CAGGAGCTGCTGTGGGCAGCCCTGCCTCTGCTTCCCCATCTGC |
| | | CTCTGGAGAGCTTTCTCCAGCTCAGCCCTCACCAGATCCAGGCC |
| | | CTGGAGGATAGCTGGCCAGTAGCAGATCTTGGGCCGGGACACG |
| | | CCCGACATGTGCTTCGTAGCCTAGTAAACCAGAGCATGGAGGA |
| | | TGGGGAGGAGCAGGTGCTCAGGCTTGGGTCCCTCGCCTGTTTC |
| | | CTGAGTCCTGAGGAGCTACAGAGTCTGGTGCCCTTGAGTGATC |
| | | CAATGGGGCCTGTAGAACAGGGTCTGCTGGAATGTGCGGCCAA |
| | | TGGGACCCTCAGCCCAGAAGGACGGGTGGCATATGAACTTCTG |
| | | GGAGTGTTGCGTTCATCTGGAGGAACTGTCTTAAGCCCCCGAGA |
| | | GCTGAGGGTCTGGGCACCTCTCTTTCCCCAGCTGGGCCTCCGC |
| | | TTCCTGCAGGAGCTCTCAGAGACCCAGCTTAGAGCCATGCTTCC |
| | | TGCCCTACAGGGAGCCAGTGTCACACCTGCCCAGGCTGTTCTG |
| | | TTGTTTGGAAGGCTCCTTCCTAAGCATGATCTGTCCCTGGAGGA |
| | | ACTCTGCTCCCTGCACCCTCTCCTGCCAGGTCTCAGCCCCCAGA |
| | | CACTCCAGGCCATCCCTAAGAGAGTTCTGGTTGGTGCTTGTTCC |
| | | TGCCTGGGCCCTGAACTGTCAAGGCTTTCAGCTTGCCAGATTGC |
| | | AGCTCTGCTGCAGACCTTTCGGGTAAAAGATGGTGTTAAAAATA |
| | | TGGGTGCAGCAGGTGCCGGCTCAGCCGTGTGCATTCCTGGGCA |
| | | GCCCACCACTTGGCCAGACTGCCTGCTTCCCCTGCTCCCATTAA |
| | | AGCTGCTACAGCTGGACGCTGCAGCTCTTCTGGCAAACCGAAG |
| | | ACTCTATCGGCAGCTGCCTTGGTCTGAGCAACAGGCACAGTTTC |
| | | TCTGGAAGAAAATGCAAGTGCCTACCAACCTGAGCCTGAGGAAT |
| | | CTGCAGGCTCTGGGCAACTTGGCAGGAGGCATGACCTGCGAGT |
| | | TTCTGCAGCAGATCAGCTCAATGGTTGACTTTCTTGATGTGGTAC |
| | | ACATGCTCTACCAGCTGCCCACTGGTGTTCGAGAGAGCCTGCG |
| | | GGCCTGTATCTGGACAGAGCTACAGCGGAGGATGACAATGCCA |
| | | GAGCCAGAGCTGACCACCCTAGGGCCAGAACTGAGTGAACTTG |
| | | ACACAAAGCTACTCCTGGACTTGCCGATCCAGCTGATGGACAGA |
| | | TTGTCCAATGATTCCATTATGTTGGTGGTGGAGATGGTCCAAGG |
| | | CGCTCCAGAGCAGCTGCTGGCACTGACCCCACTCCACCAGACA |
| | | GCCTTGGCAGAGCGAGCACTTAAAAACCTGGCTCCAAAGGAGA |
| | | CCCCAATCTCCAAAGAAGTGCTGGAGACACTGGGCCCCTTGGTT |
| | | GGATTCCTGGGAATAGAGAGCACGCGACGGATCCCTTTACCCAT |
| | | TCTACTGTCTCATCTCAGTCAGCTGCAGGGCTTCTGCCTAGGAG |
| | | AGACATTTGCCACAGAGCTGGGATGGCTGCTGTTGCAGGAGCC |
| | | TGTTCTTGGAAAACCAGAATTGTGGAGCCAGGATGAAATAGAGC |
| | | AAGCTGGACGCCTAGTATTCACTCTGTCTGCTGAGGCTATTTCC |
| | | TCGATCCCCAGGGAGGCTTTGGGCCCAGAGACACTGGAGAGGC |
| | | TTCTGGGAAAGCATCAAAGCTGGGAGCAGAGCAGAGTGGGCCA |
| | | TCTGTGTGGGGAGTCACAGCTTGCCCACAAGAAAGCAGCTCTG |
| | | GTAGCTGGGATTGTGCATCCAGCTGCTGAGGGTCTCCAAGAGC |
| | | CTGTACCAAACTGTGCAGACATACGGGGAACCTTCCCAGCGGC |
| | | CTGGTCTGCGACACAAATCTCAGAGATGGAACTCTCAGACTTTG |
| | | AAGACTGCCTGTCACTATTTGCTGGAGATCCAGGACTTGGTCCT |
| | | GAGGAACTACGGGCAGCCATGGGCAAGGCCAAGCAGTTGTGG |
| | | GGTCCCCCTCGAGGATTCCGTCCTGAGCAGATCTTGCAGCTGG |
| | | GCCGTCTCCTGATAGGTCTAGGAGAACGGGAACTGCAGGAGCT |
| | | TACCTTGGTGGACTGGGGTGTGCTGAGCAGCCTGGGGCAAATA |
| | | GATGGCTGGAGTTCCATGCAGCTCCGAGCCGTGGTCTCCAGTT |
| | | TCCTAAGGCAGAGTGGTCGGCATGTGAGCCACCTGGACTTCATT |
| | | TATCTGACAGCACTGGGTTACACAGTCTGTGGATTGCGACCAGA |
| | | GGAGTTACAGCACATCAGCAGTTGGGAGTTTAGCCAAGCAGCTC |
| | | TCTTCCTGGGTAGCTTGCATCTCCCGTGCTCTGAGGAACAGCTG |
| | | GAAGTTCTGGCCTATCTCCTTGTGTTGCCTGGTGGCTTTGGCCC |
| | | AGTCAGTAACTGGGGGCCTGAGATCTTCACTGAAATTGGCACAA |
| | | TAGCAGCTGGCATCCCAGACCTGGCTCTTTCAGCATTACTGCGG |
| | | GGACAGATCCAAGGCCTGACTCCTCTTGCCATTTCTGTCATTCC |
| | | TGCTCCCAAGTTTGCAGTGGTCTTCAACCCCATCCAGTTATCTA |
| | | GTCTCACCAGGGGTCAGGCCGTAGCTGTTACTCCTGAACAGCT |
| | | GGCCTATCTGAGTCCTGAGCAGCGGCGAGCAGTTGCATGGGCC |
| | | CAACACGAAGGGAAGGAGATCCCAGAGCAGCTGGGTCGAAACT |
| | | CAGCCTGGGGTCTCTACGACTGGTTCCAAGCCTCCTGGGCCCT |
| | | GGCATTGCCCGTCAGCATTTTTGGCCACCTATTATGA |

### Expression of Stereocilin in Mammalian Cells

Mutations in STRC have been linked to sensorineural hearing loss. The compositions and methods described herein can be used to induce or increase the expression of WT stereocilin by administering to a subject or contacting a cell with a first nucleic acid vector that contains a polynucleotide encoding an N-terminal portion of a stereocilin protein and a second nucleic acid vector that contains a polynucleotide encoding a C-terminal portion of a stereocilin protein. In order to utilize nucleic acid vectors for therapeutic application in the treatment of sensorineural hearing loss, they can be directed to the interior of the cell, and, in particular, to specific cell types. A wide array of methods has been established for the delivery of proteins to mammalian cells and for the stable expression of genes encoding proteins in mammalian cells.

### Polynucleotides Encoding Stereocilin

One platform that can be used to achieve therapeutically effective intracellular concentrations of stereocilin in mammalian cells is via the stable expression of the gene encoding stereocilin (e.g., by integration into the nuclear or mitochondrial genome of a mammalian cell, or by episomal concatemer formation in the nucleus of a mammalian cell). The gene is a polynucleotide that encodes the primary amino acid sequence of the corresponding protein. In order to introduce exogenous genes into a mammalian cell, genes can be incorporated into a vector. Vectors can be introduced into a cell by a variety of methods, including transformation, transfection, transduction, direct uptake, projectile bombardment, and by encapsulation of the vector in a liposome. Examples of suitable methods of transfecting or transforming cells include calcium phosphate precipitation, electroporation, microinjection, infection, lipofection and direct uptake. Such methods are described in more detail, for example, in Green, et al., Molecular Cloning: A Laboratory Manual, Fourth Edition (Cold Spring Harbor University Press, New York 2014); and Ausubel, et al., Current Protocols in Molecular Biology (John Wiley & Sons, New York 2015), the disclosures of each of which are incorporated herein by reference.

STRC can also be introduced into a mammalian cell by targeting vectors containing portions of a gene encoding a stereocilin protein to cell membrane phospholipids. For example, vectors can be targeted to the phospholipids on the extracellular surface of the cell membrane by linking the vector molecule to a VSV-G protein, a viral protein with affinity for all cell membrane phospholipids. Such a construct can be produced using methods well known to those of skill in the field.

Recognition and binding of the polynucleotide encoding a stereocilin protein by mammalian RNA polymerase is important for gene expression. As such, one may include sequence elements within the polynucleotide that exhibit a high affinity for transcription factors that recruit RNA polymerase and promote the assembly of the transcription complex at the transcription initiation site. Such sequence elements include, e.g., a mammalian promoter, the sequence of which can be recognized and bound by specific transcription initiation factors and ultimately RNA polymerase. Examples of mammalian promoters have been described in Smith, et al., Mol. Sys. Biol., 3:73, online publication, the disclosure of which is incorporated herein by reference.

Polynucleotides suitable for use in the compositions and methods described herein include those that encode a stereocilin protein downstream of a mammalian promoter (e.g., a polynucleotide that encodes an N-terminal portion of a stereocilin protein downstream of a mammalian promoter). Promoters that are useful for the expression of a stereocilin protein in mammalian cells include OHC-specific promoters, such as an oncomodulin (OCM) promoter (e.g., a polynucleotide having at least 85% sequence identity (e.g., 86%, 87%, 88%, 89%, 90%, 91%, 92%, 93%, 94%, 95%, 96%, 97%, 98%, 99%, or more, sequence identity) to any one of the OCM promoter sequences listed in Table 3 (e.g., any one of SEQ ID NOs: 1-3)).

### Oncomodulin Promoters

The present inventors have discovered of a region of 1,140 base pairs (bp) located upstream of the OCM translation start site that is sufficient for driving gene expression in OHCs. The compositions and methods described herein can, thus, be used to express stereocilin in OHCs to treat subjects having or at risk of developing hearing loss (e.g., sensorineural hearing loss associated with a mutation in STRC, such as DFNB16). Since the OCM promoters described herein (e.g., an OCM promoter having at least 85% sequence identity to SEQ ID NO: 1) can be used to induce OH-specific gene expression, they can reduce or eliminate off-target expression in other inner ear cells (e.g., in cells other than OHCs), thereby improving the safety and efficacy of gene therapy by targeting STRC expression to the cells in which it is endogenously expressed and reducing toxicity associated with off-target expression.

The compositions and methods described herein include an OCM promoter listed in Table 3 (e.g., any one of SEQ ID NOs: 1-3) that is capable of expressing stereocilin specifically in OHCs, such as a polynucleotide sequence having at least 85% sequence identity (e.g., 86%, 87%, 88%, 89%, 90%, 91%, 92%, 93%, 94%, 95%, 96%, 97%, 98%, 99%, or more, sequence identity) to any one of SEQ ID NOs: 1-3.

Exemplary OCM promoter sequences are listed in Table 3.

TABLE 3
OCM promoter sequences

| SEQ | Description of promoter | Promoter sequence |
|---|---|---|
| ID NO: | sequence | |
| 1 | Murine OCM promoter | AGCAGGTTTGTTACAGAAACCTTAGTTAAGGTTTGTTGAGG |
| | sequence (1140 bp) | GTTTTTTTTCTCTCTCTCTCTCTTAATTGGCTGTCCCAATCC |
| | | ATCCTTCTATAAATAGAAAAGAGAGACAGGGAGTGTGTGTG |
| | | GTTTCATTACTAAGGTAAAGACACTTGAGCTACACACACTT |
| | | GATCCCTGAACATGAAATCTAAGAGGTTGAACGATCACAGT |
| | | TTCAGGACTATATAAGGTGGTGAAAGACCATCTGCTTCGTT |
| | | TTTCTGTTTGTTCCTACAACTCTTTCCCTCCGCTTGATTTTA |
| | | ACTCTAAATTGGTGAGTAGCTGGTGGGCTCACCAGACTCC |
| | | GAGATCCTCTTCTCTGCACGCACTGTATTAGACTTGGCACC |
| | | CGGGAGGATTTTCACCTCTGCTGCATGGGCTAATCTTCCA |
| | | CAAGGGATCTGTGGTATTGCAATCTCGGGTTGATGCATGA |
| | | CGGTGATGTTGTGTTTATAGCATGGCTAAGGTTTAGCTGCC |
| | | TATGATGATTGGTTAGGGAAGGATAATTTTTGCTAGAAGAT |
| | | TGGACTTTAGGGAAAAAAAACCCCACTTTTATTTGCTTTTAG |
| | | AATTTTAAAAGACTGGGCCATGTAGCTCAGGCTGGTTTGGA |
| | | GTTCATTATGTAGTCAAGGATGCTCTTGGACTCTTTAGCAT |
| | | CCTCCTCCTCCTCTTTTTCCTCCTCCTCCTTCTTGTTCTTCT |
| | | TCTTGTTCTTCCTCTTCCCCTTCTCTTCCCCCTTCTCTCCCT |
| | | CTTCCTCCTCTTCCTCCTCCTTCTTGTTCTTCTTCCTCTTCC |
| | | CCTTCTCCTCTCCCCCTTGTCCTCCTCTTCCTTCTCCTCCT |
| | | CCTCCTCTTCTTCTTTCTGAGTACCAAGATTGCAAGTGTGC |
| | | ACACGATGACCAGCTTGGTCTTTCTTTGTCTTTTTTTTTTAA |
| | | CTTCAATTTTGGAGTGAATTCAAGAGCAACCATGTAGTCAA |
| | | GAGGTGGCTGGAGTCTTTTCTGTATCTGGGTTTGGTTTAGT |
| | | ACTCTGCCCCATCACTTAACAGGTCCTTATGGCCACATCTT |
| | | AAAAAAATTCTAGAGATACACGGTGTCGGTGAGTGGCTGA |
| | | GAATGTGTGGTCTTCCCATTTCTCTGTCACCGTGGCTCACA |
| | | TCTTGTTTCCTCTGTTCGGCCAGGTAGAAA |
| 2 | Human OCM promoter | TTTTACCACAATAATTAAAAAGAACAGTCTAGCACAGTGCT |
| | sequence containing a | GGCCATATAAAGGCTCAATAAATGTTTGCTGAAAGTTAAAA |
| | polynucleotide located −2 kb | AAAAAAAAAAAAAAAAAAAAGCCAGGCGCAGTGGTTCATTC |
| | to +0.5 kb of the TSS of the | CTGTAATCCCAGCACTTTAGGAGGATGAGGTGGGAGAATT |
| | human OCM gene | ACTTGAGCCCAGGAGTTCGAGACCAGCCTAGGCAACATGG |
| | | CAAAACCCTGTCAAAACCCTGTCTCTCCAAAAAATATGCAT |
| | | ATTTAAAAAATTAGCCAGGCATGGTGGTGTGTGCCTGTAGT |
| | | ACCAGCTACTCGGGAGACTGAAGTGGGAGGATCGCTTGAG |
| | | CCTGGGAGGTCAAGGCTGCAATGAGCTGAGATCGTGCCAC |
| | | TGCACTCCAGCCGGGGCAACAGAGCAAGACCCTGTCACAA |
| | | CAGAAACAAAATCTTGAGGTGTCTAGTCCTGGCCTCAGCCT |
| | | CAGAATATTTGTTTCTGAACATGTTAGTTTTGGGGGTTGGG |
| | | GATGCTGGTTTGATTTCCTCCTTTTTGCCTTTTGAGTGTGTG |
| | | CAATTTATGGTATAGCTGGGAAACGTCAAAGTCAAGAGTTT |
| | | TGTAGGAAAGTCACGTCACTTAGCCCTGTCTCCTGTGCCG |
| | | GGTGAGACCTGTGTGTGCACTTGGTGACAATGGCTTTGAG |
| | | TCTGTCAACTCCAGACTGAGGTCAGCCTTACACACCCATAG |
| | | TTCCCAAAGCTGAAAACAGGCCTGCCTCCAACGGTACCTG |
| | | CTAATATCAGGGGAGCCTTTTCAGCTTACAGAGCACCCTGT |
| | | ATGTGTTTGTCTTAGTTCAGGCCACCATCTCCACCTTACCA |
| | | GGCATCTAGAACCTTCTCCACACTTTGCCAACAGGGTTCGT |
| | | TTGCAGAATTGAAATCTTAGTTAAGGTTTGTTGAAGTTTGTT |
| | | GTTGTTTTTTTTTTTTTTTTACAATTGGCTGTTCCCACCCACA |
| | | TTCCCTTGAGACATAAATAGAAAAAAAAAAAAAAAGAGGTTT |
| | | CATGAGTAAGACAAGACATTTGAGCTGCATCCACTTGATCC |
| | | TTGAAAAGGAAATCTAAGAGGTTGTAACTATCACTTTTTCTA |
| | | GCCTATATAAGGTAGGTCAGTAAGGTAGCAAAAACACATCT |
| | | GTTGTTTTGCTCCTTCAACTCTTTTTCCTGATTCTTCCTGGG |
| | | GGGAAACCGAAAACGGTGAGTAACTGGTGGACACATCAGA |
| | | CCCCAGACTCTTTTCTTCACTGCATGCATTCATATTAGGCT |
| | | CAGGTGCTTAGACTCCTGTTTTCCGGTGGCTCTGACACCT |
| | | GGAAGGATTTTAATCTCTGGGAGATGGGCTTTTCATCCATC |
| | | TGCTTCCCACCTTTCAGGACAGGTGCATGCCTTCTTCCACA |
| | | GAATGTCTGCAAGCAGCCCAAACTGTATCCTTTCCCACGTG |
| | | GAATTTGCAACATTGCATCTCTCGGGCTGCTGTAGGAAAAT |
| | | GCCAGTGCATGTGTAACATGGTTTACGGCTGCCTATGCAA |
| | | ATGACTGATTATGTCAGTATAATTTTTATAAGAAAACAATTG |
| | | AATCCTTCTTTGGGTCATTTTTTTTTTCCATTTTTGGCATGTA |
| | | TTCAAAAGAAGGCTCTGAGACAAAAAAGGCTGGGGTGTTTT |
| | | CCGTATCTGGTTTTAATTTGGATATTCTGTCCCGTCACTTAA |
| | | TACAAAACCATGCTTATCACATTTTAAAAATTCTAGACAGGC |
| | | CTGGCTCGGTGGCTTGCATCTGTCATCCCAGCACTTTGTG |
| | | AGGCCAAGGCAGGCAGATCACCTGAGGTCAGGAGCTCAA |
| | | GACCAGCCTGGCCAACATGGCAAAACCCCGTCTCTACTAA |
| | | AAACACAAAAATTAGCCAGGCATGGTAGTGCGCACCTGTA |
| | | ATCCCAGCTACTGGGAAGGCTTAGGCAGGAGAATCACTTG |
| | | AGCCCAGGAGGCGGAGGTTGCGGTGAGCCGAGATCACGC |
| | | TCTTGCACTCCAGCCTGGGTGACAGAGTGAGACTCCGTCT |
| | | TAATTTAAAAAAAAAAATAATCTAGACACACATACAGTTTCA |
| | | GTGGGCCTGGGAAGATGTGTTTCCCCTGGATGTGCACATT |
| | | CCTGTTTGTGGCTTATCGCCTCTCATTTATTCTGTGTGAGT |
| | | AGGTAGAAAATGAGCATCACGGACGTGCTCAGTGCTGACG |
| | | ACATTGCAGCAGCGCTCCAGGAATGCCGAGGTAGAGGGG |
| | | ACGTGAGGCGGGGGTGGGATTTCCTCACAGCTTTGCACCT |
| | | CCAGCGAGTCAACACAAAATCAAAATGTAGGCCAGGCGGC |
| | | CAGACGCAGTGGCTCACACCTGTAATCCCAGCACTTTGGG |
| | | AGGCCGAGGCGGGTGGATCACGAGGTCAGGAGTTCGAGA |
| | | CCAGCCTGGCCAAGATGGTGAAACCCCATCTCTACTAAAA |
| | | ATACAAAAAAATTAACCGGGCGTGGTGGTGGGTGCCTGTA |
| | | ATCCCAGCTACTCGGGAGGCTGAGGCAGAGAATTGCTTGA |
| | | ACCCGGGAGGCAGAAGTTGCAGTGAGCTGAGATCATGCCA |
| | | CTGCACTCCAGCCTGGGCA |
| 3 | Human OCM promoter | GTTCCCAAAGCTGAAAACAGGCCTGCCTCCAACGGTACCT |
| | sequence containing | GCTAATATCAGGGGAGCCTTTTCAGCTTACAGAGCACCCT |
| | regions from SEQ ID NO: 2 | GTATGTGTTTGTCTTAGTTCAGGCACCTTACCAGGCATCTA |
| | that are conserved across | GAACCTTCTCCACACTTTGCCAACAGGGTTCGTTTGCAGAA |
| | mammalian species | TTGAAATCTTAGTTAAGGTTTGTTGAAGTTTGTTGTTGTTTT |
| | | TTTTTTTTTTTTACAATTGGCTGTTCCCACCCACATTCCCTT |
| | | GAGACATAAATAGAAAAAAAAAAAAAAAGAGGTTTCATGAG |
| | | TAAGACAAGACATTTGAGCTGCATCCACTTGATCCTTGAAA |
| | | AGGAAATCTAAGAGGTTGTAACTATCACTTTTTCTAGCCTAT |
| | | ATAAGGTAGGTCAGTAAGGTAGCAAAAACACATCTGTTGTT |
| | | TTGCTCCTTCAACTCTTTTTCCTGATTCTTCCTGGGGGGAA |
| | | ACCGAAAACGGTGAGTAACTGGTGGACACATCAGACCCCA |
| | | GACTCTTTTCTTCACTGCATGCATTCATATTAGGCTCAGGT |
| | | GCTTAGACTCCTGTTTTCCGGTTTACGGCTGCCTATGCAAA |
| | | TGACTGATTATGTCAGTATAATTTTTATAAGAAAACAATTGA |
| | | ATCCTTCTTTGGGTCATTTTTTTTTTCCATTTTTGGCATGTAT |
| | | GTGCACATTCCTGTTTGTGGCTTATCGCCTCTCATTTATTCT |
| | | GTGTGAGTAGGTAGAAAATGAGCATCACGGACGTGCTCAG |
| | | TGCTGACGACATTGCAGCAGCGCTCCAGGAATGCCGAGGT |
| | | AGAGGGGACGTGAGGGGGGGGTGGGATTTCCTCACAGCT |
| | | TTGCACCTCCAGC |

The foregoing polynucleotides can be included in a nucleic acid vector and operably linked to a transgene to express the transgene specifically in OHCs. In the vectors described herein, the transgene can encode an N-terminal portion of a stereocilin protein. According to the methods described herein, a subject can be administered a composition containing one of the foregoing polynucleotides (e.g., any one the polynucleotide sequences listed in Table 3 (e.g., SEQ ID NOs: 1-3) or a polynucleotide sequence having at least 85% sequence identity thereto (e.g., 85%, 86%, 87%, 88%, 89%, 90%, 91%, 92%, 93%, 94%, 95%, 96%, 97%, 98%, 99%, or more, sequence identity any one of SEQ ID NOs: 1-3)) operably linked to a polynucleotide encoding, e.g., an N-terminal portion of a stereocilin protein (e.g., an N-terminal portion of SEQ ID NO: 4 or SEQ ID NO: 5) for the treatment of hearing loss.

Once a polynucleotide encoding stereocilin has been incorporated into the nuclear DNA of a mammalian cell, the transcription of this polynucleotide can be induced by methods known in the art. For example, expression can be induced by exposing the mammalian cell to an external chemical reagent, such as an agent that modulates the binding of a transcription factor and/or RNA polymerase to the mammalian promoter and thus regulates gene expression. The chemical reagent can serve to facilitate the binding of RNA polymerase and/or transcription factors to the mammalian promoter, e.g., by removing a repressor protein that has bound the promoter. Alternatively, the chemical reagent can serve to enhance the affinity of the mammalian promoter for RNA polymerase and/or transcription factors such that the rate of transcription of the gene located downstream of the promoter is increased in the presence of the chemical reagent. Examples of chemical reagents that potentiate polynucleotide transcription by the above mechanisms include tetracycline and doxycycline. These reagents are commercially available (Life Technologies, Carlsbad, CA) and can be administered to a mammalian cell in order to promote gene expression according to established protocols.

Other DNA sequence elements that may be included in polynucleotides for use in the compositions and methods described herein include enhancer sequences. Enhancers represent another class of regulatory elements that induce a conformational change in the polynucleotide containing the gene of interest such that the DNA adopts a three-dimensional orientation that is favorable for binding of transcription factors and RNA polymerase at the transcription initiation site. Thus, polynucleotides for use in the compositions and methods described herein include those that encode an STRC protein and additionally include a mammalian enhancer sequence. Many enhancer sequences are now known from mammalian genes, and examples include enhancers from the genes that encode mammalian globin, elastase, albumin, α-fetoprotein, and insulin. Enhancers for use in the compositions and methods described herein also include those that are derived from the genetic material of a virus capable of infecting a eukaryotic cell. Examples include the SV40 enhancer on the late side of the replication origin (bp 100-270), the cytomegalovirus early promoter enhancer, the polyoma enhancer on the late side of the replication origin, and adenovirus enhancers. Additional enhancer sequences that induce activation of eukaryotic gene transcription include the CMV enhancer and RSV enhancer. An enhancer may be spliced into a vector containing a polynucleotide encoding a protein of interest, for example, at a position 5′ or 3′ to this gene. In a preferred orientation, the enhancer is positioned at the 5′ side of the promoter, which in turn is located 5′ relative to the polynucleotide encoding a stereocilin protein.

The nucleic acid vectors described herein may include a Woodchuck Posttranscriptional Regulatory Element (WPRE). The WPRE acts at the mRNA level, by promoting nuclear export of transcripts and/or by increasing the efficiency of polyadenylation of the nascent transcript, thus increasing the total amount of mRNA in the cell. The addition of the WPRE to a vector can result in a substantial improvement in the level of transgene expression from several different promoters, both in vitro and in vivo.

In some embodiments, the nucleic acid vectors described herein include a reporter sequence, which can be useful in verifying stereocilin expression, for example, in cells and tissues (e.g., in OHCs). Reporter sequences that may be provided in a transgene include DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements that drive their expression, such as an OCM promoter, the reporter sequences provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA), and immunohistochemistry. For example, where the marker sequence is the LacZ gene, the presence of the vector carrying the signal is detected by assays for β-galactosidase activity. Where the transgene is green fluorescent protein or luciferase, the vector carrying the signal may be measured visually by color or light production in a luminometer.

### Dual Vector Expression Systems

### Overlapping Dual Vectors

One approach for expressing large proteins in mammalian cells involves the use of overlapping dual vectors. This approach is based on the use of two nucleic acid vectors, each of which contains a portion of a polynucleotide that encodes a protein of interest and has a defined region of sequence overlap with the other polynucleotide. Homologous recombination can occur at the region of overlap and lead to the formation of a single polynucleotide that encodes the full-length protein of interest (e.g., a stereocilin protein).

Overlapping dual vectors for use in the methods and compositions described herein contain at least 200 bases of overlapping sequence (e.g., at least 200 b, 300 b, 400 b, 500 b, 600 b, 700 b, 800 b, 900 b, 1.0 kilobase (kb), 1.1 kb, 1.2 kb, 1.3 kb, 1.4 kb, 1.5 kb or more of overlapping sequence). The nucleic acid vectors are designed such that the overlapping region is centered at or near a position within the stereocilin-encoding polynucleotide that corresponds to approximately half of the length of the stereocilin-encoding polynucleotide, with an equal amount of overlap on either side of the central position. The center of the overlapping region can also be chosen based on the size of the promoter and the locations of sequence elements of interest in the polynucleotide that encodes stereocilin. In some embodiments, the stereocilin-encoding polynucleotide is split in two halves of approximately equal length with some degree of overlap (e.g., 50 b, 100 b, 150 b, 200 b, 250 b, 300 b, 350 b, 400 b, 450 b, 500 b, 600 b, 700 b, 800 b, 900 b, 1 kb, 1.1 kb, 1.2 kb, 1.3 kb, 1.4 kb, 1.5 kb, or more), in which the 5′ half of the polynucleotide encodes an N-terminal portion of the stereocilin protein and the 3′ half of the polynucleotide encodes a C-terminal portion of the stereocilin protein. The nucleic acid vectors for use in the methods and compositions described herein are also designed such that approximately half of the stereocilin-encoding polynucleotide is contained within each vector (e.g., each vector contains a polynucleotide that encodes approximately half of the stereocilin protein).

In some embodiments, the first nucleic acid vector encodes an N-terminal portion of the stereocilin protein. In some embodiments, the second nucleic acid vector encodes a C-terminal portion of the stereocilin protein. In some embodiments, the stereocilin protein has the sequence of SEQ ID NO: 4 or at least 85% (e.g., at least 86%, 87%, 88%, 89%, 90%, 91%, 92%, 93%, 94%, 95%, 96%, 97%, 98%, 99%, or more) sequence identity thereto. In some embodiments, the stereocilin protein has the sequence of SEQ ID NO: 5 or at least 85% (e.g., at least 86%, 87%, 88%, 89%, 90%, 91%, 92%, 93%, 94%, 95%, 96%, 97%, 98%, 99%, or more) sequence identity thereto. In some embodiments, the polynucleotide that encodes a full-length human stereocilin protein has the sequence of SEQ ID NO: 6 or is a variant thereof having at least 85% (e.g., at least 86%, 87%, 88%, 89%, 90%, 91%, 92%, 93%, 94%, 95%, 96%, 97%, 98%, 99%, or more) sequence identity to SEQ ID NO: 6. In some embodiments, the polynucleotide that encodes a full-length murine stereocilin protein has the sequence of SEQ ID NO: 7 or is a variant thereof having at least 85% (e.g., at least 86%, 87%, 88%, 89%, 90%, 91%, 92%, 93%, 94%, 95%, 96%, 97%, 98%, 99%, or more) sequence identity to SEQ ID NO: 7.

One exemplary overlapping dual vector system includes a first nucleic acid vector containing an OCM promoter described hereinabove (e.g., an OCM promoter having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 1-3) operably linked to polynucleotide encoding an N-terminal portion of a stereocilin protein (e.g., an N-terminal portion of SEQ ID NO: 4 or SEQ ID NO: 5) including 500 b immediately 3′ of the position selected as the central position; and a second nucleic acid vector containing the C-terminal portion of the polynucleotide encoding the stereocilin protein, which includes 500 b immediately 5′ of the position selected as the central position, and a poly(A) sequence (e.g., a bovine growth hormone (bGH) poly(A) signal sequence). The nucleic acid vectors can optionally contain STRC untranslated regions (UTRs). In some embodiments, the OCM promoter is a polynucleotide having the sequence of SEQ ID NO: 1 or a variant having at least 85% (e.g., at least 86%, 87%, 88%, 89%, 90%, 91%, 92%, 93%, 94%, 95%, 96%, 97%, 98%, 99%, or more) sequence identity to SEQ ID NO: 1. In some embodiments, the OCM promoter is a polynucleotide having the sequence of SEQ ID NO: 2 or a variant having at least 85% (e.g., at least 86%, 87%, 88%, 89%, 90%, 91%, 92%, 93%, 94%, 95%, 96%, 97%, 98%, 99%, or more) sequence identity SEQ ID NO: 2. In some embodiments, the OCM promoter is a polynucleotide having the sequence of SEQ ID NO: 3 or a variant having at least 85% (e.g., at least 86%, 87%, 88%, 89%, 90%, 91%, 92%, 93%, 94%, 95%, 96%, 97%, 98%, 99%, or more) sequence identity SEQ ID NO: 3.

In some embodiments, the first member of the dual vector system includes the OCM promoter of SEQ ID NO:1 (also represented by nucleotides 225-1364 of SEQ ID NO: 43) operably linked to nucleotides that encode an N-terminal portion of a stereocilin protein. In certain embodiments, the nucleotide sequence that encodes an N-terminal portion of a stereocilin protein is nucleotides 1375-4574 of SEQ ID NO: 43. The nucleotide sequences that encode an N-terminal portion of a stereocilin protein can be partially or fully codon-optimized for expression. In particular embodiments, the first member of the dual vector system includes nucleotides 225-4574 of SEQ ID NO: 43 flanked on each of the 5′ and 3′ sides by an inverted terminal repeat. In some embodiments, the flanking inverted terminal repeats are any variant of AAV2 inverted terminal repeats that can be encapsidated by a plasmid that carries the AAV2 Rep gene. In certain embodiments, the 5′ flanking inverted terminal repeat has a sequence corresponding to nucleotides 1-130 of SEQ ID NO: 43 or a sequence having at least 80% sequence identity (at least 80%, 81%, 82%, 83%, 84%, 85%, 86%, 87%, 88%, 89%, 90%, 91%, 92%, 93%, 94%, 95%, 96%, 97%, 98%, or 99% sequence identity) thereto; and the 3′ flanking inverted terminal repeat has a sequence corresponding to nucleotides 4662-4791 of SEQ ID NO: 43 or a sequence having at least 80% sequence identity (at least 80%, 81%, 82%, 83%, 84%, 85%, 86%, 87%, 88%, 89%, 90%, 91%, 92%, 93%, 94%, 95%, 96%, 97%, 98%, or 99% sequence identity) thereto. It will be understood by those of skill in the art that, for any given pair of inverted terminal repeat sequences in a transfer plasmid that is used to create the viral vector (typically by transfecting cells with that plasmid together with other plasmids carrying the necessary AAV genes for viral vector formation) (e.g., SEQ ID NO: 43), that the corresponding sequence in the viral vector can be altered due to the ITRs adopting a “flip” or “flop” orientation during recombination. Thus, the sequence of the ITR in the transfer plasmid is not necessarily the same sequence that is found in the viral vector prepared therefrom. However, in some very specific embodiments, the first member of the dual vector system includes nucleotides 1-4791 of SEQ ID NO: 43.

In some embodiments, the second member of the dual vector system includes nucleotides that encode the C-terminal portion of the stereocilin protein immediately followed by a stop codon. In certain embodiments, the nucleotide sequence that encodes the C-terminal amino acids of the stereocilin protein is nucleotides 211-3440 of SEQ ID NO: 44. The nucleotide sequences that encode the C-terminal portion of the STRC protein can be partially or fully codon-optimized for expression. In some embodiments, the second member of the dual vector system includes a WPRE sequence corresponding to nucleotides 3452-3999 of SEQ ID NO: 44. In some embodiments, the second member of the dual vector system includes the poly(A) sequence corresponding to nucleotides 4012-4219 of SEQ ID NO: 44. In particular embodiments, the second member of the dual vector system includes nucleotides 211-4219 of SEQ ID NO: 44 flanked on each of the 5′ and 3′ sides by an inverted terminal repeat. In some embodiments, the flanking inverted terminal repeats are any variant of AAV2 inverted terminal repeats that can be encapsidated by a plasmid that carries the AAV2 Rep gene. In certain embodiments, the 5′ flanking inverted terminal repeat has a sequence corresponding to nucleotides 1-130 of SEQ ID NO: 44 or a sequence having at least 80% sequence identity (at least 80%, 81%, 82%, 83%, 84%, 85%, 86%, 87%, 88%, 89%, 90%, 91%, 92%, 93%, 94%, 95%, 96%, 97%, 98%, or 99% sequence identity) thereto; and the 3′ flanking inverted terminal repeat has a sequence corresponding to nucleotides 4307-4436 of SEQ ID NO: 44 or a sequence having at least 80% sequence identity (at least 80%, 81%, 82%, 83%, 84%, 85%, 86%, 87%, 88%, 89%, 90%, 91%, 92%, 93%, 94%, 95%, 96%, 97%, 98%, or 99% sequence identity) thereto. It will be understood by those of skill in the art that, for any given pair of inverted terminal repeat sequences in a transfer plasmid that is used to create the viral vector (typically by transfecting cells with that plasmid together with other plasmids carrying the necessary AAV genes for viral vector formation) (e.g., SEQ ID NO: 44), that the corresponding sequence in the viral vector can be altered due to the ITRs adopting a “flip” or “flop” orientation during recombination. Thus, the sequence of the ITR in the transfer plasmid is not necessarily the same sequence that is found in the viral vector prepared therefrom. However, in some very specific embodiments, the first member of the dual vector system includes nucleotides 1-4436 of SEQ ID NO: 44.

Transfer plasmids that may be used to produce nucleic acid vectors for use in the compositions and methods described herein are provided in Tables 4 and 5. A transfer plasmid (e.g., a plasmid containing a DNA sequence to be delivered by a nucleic acid vector, e.g., to be delivered by an AAV) may be co-delivered into producer cells with a helper plasmid (e.g., a plasmid providing proteins necessary for AAV manufacture) and a rep/cap plasmid (e.g., a plasmid that provides AAV capsid proteins and proteins that insert the transfer plasmid DNA sequence into the capsid shell) to produce a nucleic acid vector (e.g., an AAV vector) for administration. Nucleic acid vectors (e.g., a nucleic acid vector (e.g., an AAV vector) containing a polynucleotide encoding an N-terminal portion of a stereocilin protein and a nucleic acid vector (e.g., an AAV vector) containing a polynucleotide encoding a C-terminal portion a stereocilin protein) can be combined (e.g., in a single formulation) prior to administration.

Transfer plasmids that may be used to produce nucleic acid vectors (e.g., AAV vectors) for co-formulation or co-administration (e.g., administration simultaneously or sequentially) in a overlapping dual vector system are provided in Table 4 (SEQ ID NO: 43 and SEQ ID NO: 44).

TABLE 4
Transfer plasmids designed to produce overlapping dual vectors

| SEQ | | |
|---|---|---|
| ID | | |
| NO. | Description | Plasmid Sequence |
| 43 | Plasmid P959 | CTGCGCGCTCGCTCGCTCACTGAGGCCGCCCGGGCAAAGCCCGG |
| | 5′ ITR at nucleotide | GCGTCGGGCGACCTTTGGTCGCCCGGCCTCAGTGAGCGAGCGAG |
| | positions 1-130 | CGCGCAGAGAGGGAGTGGCCAACTCCATCACTAGGGGTTCCTTGT |
| | Murine OCM promoter at | AGTTAATGATTAACCCGCCATGCTACTTATCTACGTAGCCATGCTC |
| | nucleotide positions | TAGGAAGATCGGAATTCGCCCTTAAGCTAGCGGCGCGCCACCGGT |
| | 225-1364 | AGCAGGTTTGTTACAGAAACCTTAGTTAAGGTTTGTTGAGGGTTTT |
| | N-terminal STRC coding | TTTTCTCTCTCTCTCTCTTAATTGGCTGTCCCAATCCATCCTTCTAT |
| | sequence at nucleotide | AAATAGAAAAGAGAGACAGGGAGTGTGTGTGGTTTCATTACTAAG |
| | positions 1375-4574 | GTAAAGACACTTGAGCTACACACACTTGATCCCTGAACATGAAATC |
| | (including overlap at | TAAGAGGTTGAACGATCACAGTTTCAGGACTATATAAGGTGGTGAA |
| | nucleotide positions | AGACCATCTGCTTCGTTTTTCTGTTTGTTCCTACAACTCTTTCCCTC |
| | 4075-4574 with P724) | CGCTTGATTTTAACTCTAAATTGGTGAGTAGCTGGTGGGCTCACCA |
| | 3′ ITR at nucleotide | GACTCCGAGATCCTCTTCTCTGCACGCACTGTATTAGACTTGGCAC |
| | positions 4662-4791 | CCGGGAGGATTTTCACCTCTGCTGCATGGGCTAATCTTCCACAAG |
| | | GGATCTGTGGTATTGCAATCTCGGGTTGATGCATGACGGTGATGT |
| | | TGTGTTTATAGCATGGCTAAGGTTTAGCTGCCTATGATGATTGGTT |
| | | AGGGAAGGATAATTTTTGCTAGAAGATTGGACTTTAGGGAAAAAAA |
| | | ACCCCACTTTTATTTGCTTTTAGAATTTTAAAAGACTGGGCCATGTA |
| | | GCTCAGGCTGGTTTGGAGTTCATTATGTAGTCAAGGATGCTCTTGG |
| | | ACTCTTTAGCATCCTCCTCCTCCTCTTTTTCCTCCTCCTCCTTCTTG |
| | | TTCTTCTTCTTGTTCTTCCTCTTCCCCTTCTCTTCCCCCTTCTCTCC |
| | | CTCTTCCTCCTCTTCCTCCTCCTTCTTGTTCTTCTTCCTCTTCCCCT |
| | | TCTCCTCTCCCCCTTGTCCTCCTCTTCCTTCTCCTCCTCCTCCTCTT |
| | | CTTCTTTCTGAGTACCAAGATTGCAAGTGTGCACACGATGACCAGC |
| | | TTGGTCTTTCTTTGTCTTTTTTTTTTAACTTCAATTTTGGAGTGAATT |
| | | CAAGAGCAACCATGTAGTCAAGAGGTGGCTGGAGTCTTTTCTGTAT |
| | | CTGGGTTTGGTTTAGTACTCTGCCCCATCACTTAACAGGTCCTTAT |
| | | GGCCACATCTTAAAAAAATTCTAGAGATACACGGTGTCGGTGAGTG |
| | | GCTGAGAATGTGTGGTCTTCCCATTTCTCTGTCACCGTGGCTCACA |
| | | TCTTGTTTCCTCTGTTCGGCCAGGTAGAAAGGCGGCCGCCATGGC |
| | | TCTGAGCCTCCAGCCCCAGCTGCTCCTTCTCCTGTCGCTCCTGCC |
| | | GCAGGAAGTGACTTCAGCCCCTACTGGGCCTCAGTCTTTGGATGC |
| | | TGGTCTCTCCCTTCTGAAGTCATTCGTAGCCACTCTGGACCAAGCT |
| | | CCTCAGCGTTCCCTCAGCCAGTCACGGTTCTCTGCGTTCCTGGCC |
| | | AACATTTCTTCATCCTTCCAGCTTGGGAGGATGGGGGAGGGACCG |
| | | GTGGGAGAGCCCCCACCTCTCCAGCCCCCTGCACTTCGACTTCAT |
| | | GATTTCCTCGTGACACTGAGAGGTAGCCCAGACTGGGAGCCAATG |
| | | CTAGGGCTTCTGGGAGATGTGCTGGCACTCCTGGGACAGGAACA |
| | | GACTCCCCGGGACTTTTTGGTGCACCAGGCAGGTGTACTGGGTG |
| | | GACTTGTAGAGGCATTGTTGGGAGCGTTAGTTCCTGGAGGCCCCC |
| | | CTGCCCCCACTCGACCCCCATGCACCCGTGATGGCCCTTCTGACT |
| | | GTGTCCTGGCTGCTGATTGGTTGCCTTCTCTGATGTTGTTATTAGA |
| | | GGGTACACGCTGGCAGGCCCTGGTGCAGTTGCAGCCCAGTGTGG |
| | | ACCCAACCAATGCCACAGGTCTTGATGGTAGAGAGCCAGCTCCTC |
| | | ACTTTTTACAGGGTCTGCTGGGCTTGCTTACCCCAGCAGGAGAGT |
| | | TGGGCTCTGAGGAGGCTCTTTGGGGTGGTCTGCTGCGCACAGTG |
| | | GGGGCCCCCCTCTATGCTGCCTTCCAGGAGGGGCTACTGCGAGT |
| | | CACTCATTCTCTGCAAGATGAGGTCTTTTCTATTATGGGACAGCCA |
| | | GAGCCTGATGCCAGTGGGCAGTGCCAGGGAGGCAACCTTCAACA |
| | | GCTGCTTTTATGGGGCATGCGGAACAACCTTTCTTGGGACGCCCG |
| | | AGCACTGGGTTTTCTATCTGGATCACCACCTCCACCCCCTGCTCTC |
| | | CTGCACTGCCTGAGCAGAGGTGTGCCTCTGCCCAGGGCTTCCCA |
| | | GCCTGCGGCTCACATCAGCCCTCGACAGCGGCGAGCCATCTCTG |
| | | TGGAGGCCCTCTGCGAGAACCACTCAGGCCCAGAGCCACCCTAC |
| | | AGCATCTCCAACTTCTCCATCTACTTGCTCTGCCAGCACATCAAGC |
| | | CTGCCACCCCGCGGCCCCCTCCTACCACCCCACGGCCTCCTCCT |
| | | ACCACCCCACAGCCCCCTCCTACCACTACACAGCCCATTCCTGAC |
| | | ACTACACAGCCCCCTCCTGTCACCCCAAGGCCTCCTCCTACCACC |
| | | CCACAACCCCCTCCTAGCACAGCTGTCATCTGCCAGACAGCTGTA |
| | | TGGTACGCAGTCTCGTGGGCACCAGGTGCCCGAGGTTGGCTCCA |
| | | AGCCTGCCATGATCAGTTTCCTGATCAATTTCTGGATATGATCTGC |
| | | GGCAACCTCTCATTTTCAGCCCTGTCTGGCCCCAGTCGTCCTTTG |
| | | GTAAAGCAGCTCTGTGCTGGCTTGCTCCCACCCCCCACTAGCTGT |
| | | CCACCAGGCCTGATCCCTGTGCCCCTCACCCCAGAAATATTCTGG |
| | | GGCTGTTTCCTGGAGAATGAGACACTGTGGGCTGAACGGTTGTGT |
| | | GTGGAGGACAGTCTGCAGGCTGTGCCCCCGAGGAACCAGGCTTG |
| | | GGTTCAGCATGTGTGTCGGGGCCCCACCTTGGACGCCACTGATTT |
| | | TCCACCGTGCCGCGTTGGACCCTGTGGGGAACGCTGCCCAGATG |
| | | GGGGCAGCTTCCTGCTCATGGTCTGTGCCAATGACACTCTGTATG |
| | | AAGCCTTGGTTCCCTTCTGGGCTTGGCTAGCAGGCCAATGCAGAA |
| | | TTAGTCGTGGAGGAAATGATACTTGCTTTCTAGAAGGCATGCTGG |
| | | GCCCCTTGTTGCCCTCTCTGCCCCCTCTGGGACCATCCCCACTCT |
| | | GTCTGGCTCCTGGTCCTTTTCTGCTTGGCATGTTATCCCAGTTGCC |
| | | ACGCTGTCAGTCCTCCGTGCCAGCCCTCGCCCACCCCACGCGCC |
| | | TACATTACCTCCTGCGCCTACTGACCTTCCTTCTGGGTCCAGGGA |
| | | CTGGGGGTGCCGAGACGCAGGGGATGTTAGGTCAAGCCCTGCTG |
| | | CTCTCTAGTCTCCCAGACAACTGTTCATTCTGGGATGCCTTCCGCC |
| | | CAGAGGGCCGGAGAAGTGTACTGAGGACAGTCGGAGAGTACTTG |
| | | CAGCGGGAAGAGCCAACCCCACCAGGCTTAGACTCCTCCCTCAG |
| | | CCTCGGCTCTGGTATGAGCAAGATGGAGCTTCTGTCCTGCTTCAG |
| | | TCCTGTACTGTGGGATCTACTCCAGAGAGAGAAGAGCGTTTGGGC |
| | | CCTGAGGACCCTGGTGAAGGCCTACCTGCGCATGCCTCCAGAAG |
| | | ACCTTCAGCAGCTTGTGCTTTCAGCAGAGATGGAGGCTGCACAGG |
| | | GCTTCCTGACGCTCATGCTTCGTTCCTGGGCTAAGCTGAAGGTTC |
| | | AACCATCCGAGGAGCAGGCCATGGGCCGCCTGACAGCCTTGCTG |
| | | CTCCAGCGGTACCCACGCCTCACCTCCCAACTCTTTATCGACATGT |
| | | CACCGCTCATCCCCTTCCTGGCTGTCCCTGACCTCATGCGCTTCC |
| | | CACCGTCCCTTTTGGCCAACGACAGTGTCCTGGCTGCCATCAGGG |
| | | ATCACAGCTCAGGAATGAAGCCTGAACAGAAGGAGGCCCTGGCAA |
| | | AACGACTGCTGGCCCCTGAGCTGTTTGGAGAAGTGCCTGATTGGC |
| | | CCCAGGAGCTGCTGTGGGCAGCCCTGCCTCTGCTTCCCCATCTGC |
| | | CTCTGGAGAGCTTTCTCCAGCTCAGCCCTCACCAGATCCAGGCCC |
| | | TGGAGGATAGCTGGCCAGTAGCAGATCTTGGGCCGGGACACGCC |
| | | CGACATGTGCTTCGTAGCCTAGTAAACCAGAGCATGGAGGATGGG |
| | | GAGGAGCAGGTGCTCAGGCTTGGGTCCCTCGCCTGTTTCCTGAGT |
| | | CCTGAGGAGCTACAGAGTCTGGTGCCCTTGAGTGATCCAATGGGG |
| | | CCTGTAGAACAGGGTCTGCTGGAATGTGCGGCCAATGGGACCCTC |
| | | AGCCCAGAAGGACGGGTGGCATATGAACTTCTGGGAGTGTTGCGT |
| | | TCATCTGGAGGAACTGTCTTAAGCCCCCGAGAGCTGAGGGTCTGG |
| | | GCACCTCTCTTTCCCCAGCTGGGCCTCCGCTTCCTGCAGGAGCTC |
| | | TCAGAGACCCAGCTTAGAGCCATGCTTCCTGCCCTACAGGGAGCC |
| | | AGTGTCACACCCTCGAGTTAAGGGCGAATTCCCGATAAGGATCTT |
| | | CCTAGAGCATGGCTACGTAGATAAGTAGCATGGGGGGTTAATCAT |
| | | TAACTACAAGGAACCCCTAGTGATGGAGTTGGCCACTCCCTCTCT |
| | | GCGCGCTCGCTCGCTCACTGAGGCCGGGCGACCAAAGGTCGCCC |
| | | GACGCCCGGGCTTTGCCCGGGGGGCCTCAGTGAGCGAGCGAGC |
| | | GCGCAGCCTTAATTAACCTAATTCACTGGCCGTCGTTTTACAACGT |
| | | CGTGACTGGGAAAACCCTGGCGTTACCCAACTTAATCGCCTTGCA |
| | | GCACATCCCCCTTTCGCCAGCTGGCGTAATAGCGAAGAGGCCCG |
| | | CACCGATCGCCCTTCCCAACAGTTGCGCAGCCTGAATGGCGAATG |
| | | GGACGCGCCCTGTAGCGGCGCATTAAGCGCGGCGGGTGTGGTG |
| | | GTTACGCGCAGCGTGACCGCTACACTTGCCAGCGCCCTAGCGCC |
| | | CGCTCCTTTCGCTTTCTTCCCTTCCTTTCTCGCCACGTTCGCCGGC |
| | | TTTCCCCGTCAAGCTCTAAATCGGGGGCTCCCTTTAGGGTTCCGA |
| | | TTTAGTGCTTTACGGCACCTCGACCCCAAAAAACTTGATTAGGGTG |
| | | ATGGTTCACGTAGTGGGCCATCGCCCTGATAGACGGTTTTTCGCC |
| | | CTTTGACGTTGGAGTCCACGTTCTTTAATAGTGGACTCTTGTTCCA |
| | | AACTGGAACAACACTCAACCCTATCTCGGTCTATTCTTTTGATTTAT |
| | | AAGGGATTTTGCCGATTTCGGCCTATTGGTTAAAAAATGAGCTGAT |
| | | TTAACAAAAATTTAACGCGAATTTTAACAAAATATTAACGCTTACAA |
| | | TTTAGGTGGCACTTTTCGGGGAAATGTGCGCGGAACCCCTATTTG |
| | | TTTATTTTTCTAAATACATTCAAATATGTATCCGCTCATGAGACAATA |
| | | ACCCTGATAAATGCTTCAATAATATTGAAAAAGGAAGAGTATGAGC |
| | | CATATTCAACGGGAAACGTCGAGGCCGCGATTAAATTCCAACATG |
| | | GATGCTGATTTATATGGGTATAAATGGGCTCGCGATAATGTCGGG |
| | | CAATCAGGTGCGACAATCTATCGCTTGTATGGGAAGCCCGATGCG |
| | | CCAGAGTTGTTTCTGAAACATGGCAAAGGTAGCGTTGCCAATGAT |
| | | GTTACAGATGAGATGGTCAGACTAAACTGGCTGACGGAATTTATGC |
| | | CTCTTCCGACCATCAAGCATTTTATCCGTACTCCTGATGATGCATG |
| | | GTTACTCACCACTGCGATCCCCGGAAAAACAGCATTCCAGGTATTA |
| | | GAAGAATATCCTGATTCAGGTGAAAATATTGTTGATGCGCTGGCAG |
| | | TGTTCCTGCGCCGGTTGCATTCGATTCCTGTTTGTAATTGTCCTTTT |
| | | AACAGCGATCGCGTATTTCGTCTTGCTCAGGCGCAATCACGAATG |
| | | AATAACGGTTTGGTTGATGCGAGTGATTTTGATGACGAGCGTAATG |
| | | GCTGGCCTGTTGAACAAGTCTGGAAAGAAATGCATAAACTTTTGCC |
| | | ATTCTCACCGGATTCAGTCGTCACTCATGGTGATTTCTCACTTGAT |
| | | AACCTTATTTTTGACGAGGGGAAATTAATAGGTTGTATTGATGTTG |
| | | GACGAGTCGGAATCGCAGACCGATACCAGGATCTTGCCATCCTAT |
| | | GGAACTGCCTCGGTGAGTTTTCTCCTTCATTACAGAAACGGCTTTT |
| | | TCAAAAATATGGTATTGATAATCCTGATATGAATAAATTGCAGTTTC |
| | | ATTTGATGCTCGATGAGTTTTTCTAACTGTCAGACCAAGTTTACTCA |
| | | TATATACTTTAGATTGATTTAAAACTTCATTTTTAATTTAAAAGGATC |
| | | TAGGTGAAGATCCTTTTTGATAATCTCATGACCAAAATCCCTTAACG |
| | | TGAGTTTTCGTTCCACTGAGCGTCAGACCCCGTAGAAAAGATCAAA |
| | | GGATCTTCTTGAGATCCTTTTTTTCTGCGCGTAATCTGCTGCTTGC |
| | | AAACAAAAAAACCACCGCTACCAGCGGTGGTTTGTTTGCCGGATC |
| | | AAGAGCTACCAACTCTTTTTCCGAAGGTAACTGGCTTCAGCAGAGC |
| | | GCAGATACCAAATACTGTTCTTCTAGTGTAGCCGTAGTTAGGCCAC |
| | | CACTTCAAGAACTCTGTAGCACCGCCTACATACCTCGCTCTGCTAA |
| | | TCCTGTTACCAGTGGCTGCTGCCAGTGGCGATAAGTCGTGTCTTA |
| | | CCGGGTTGGACTCAAGACGATAGTTACCGGATAAGGCGCAGCGG |
| | | TCGGGCTGAACGGGGGGTTCGTGCACACAGCCCAGCTTGGAGCG |
| | | AACGACCTACACCGAACTGAGATACCTACAGCGTGAGCTATGAGA |
| | | AAGCGCCACGCTTCCCGAAGGGAGAAAGGCGGACAGGTATCCGG |
| | | TAAGCGGCAGGGTCGGAACAGGAGAGCGCACGAGGGAGCTTCCA |
| | | GGGGGAAACGCCTGGTATCTTTATAGTCCTGTCGGGTTTCGCCAC |
| | | CTCTGACTTGAGCGTCGATTTTTGTGATGCTCGTCAGGGGGGCGG |
| | | AGCCTATGGAAAAACGCCAGCAACGCGGCCTTTTTACGGTTCCTG |
| | | GCCTTTTGCTGGCCTTTTGCTCACATGTTCTTTCCTGCGTTATCCC |
| | | CTGATTCTGTGGATAACCGTATTACCGCCTTTGAGTGAGCTGATAC |
| | | CGCTCGCCGCAGCCGAACGACCGAGCGCAGCGAGTCAGTGAGCG |
| | | AGGAAGCGGAAGAGCGCCCAATACGCAAACCGCCTCTCCCCGCG |
| | | CGTTGGCCGATTCATTAATGCAGCTGGCACGACAGGTTTCCCGAC |
| | | TGGAAAGCGGGCAGTGAGCGCAACGCAATTAATGTGAGTTAGCTC |
| | | ACTCATTAGGCACCCCAGGCTTTACACTTTATGCTTCCGGCTCGTA |
| | | TGTTGTGTGGAATTGTGAGCGGATAACAATTTCACACAGGAAACAG |
| | | CTATGACCATGATTACGCCAGATTTAATTAAGGCCTTAATTAGG |
| 44 | Plasmid P724 | CTGCGCGCTCGCTCGCTCACTGAGGCCGCCCGGGCAAAGCCCG |
| | 5′ ITR at nucleotide | GGCGTCGGGCGACCTTTGGTCGCCCGGCCTCAGTGAGCGAGCG |
| | positions 1-130 | AGCGCGCAGAGAGGGAGTGGCCAACTCCATCACTAGGGGTTCCT |
| | C-terminal STRC coding | TGTAGTTAATGATTAACCCGCCATGCTACTTATCTACGTAGCCATG |
| | sequence at nucleotide | CTCTAGGAAGATCGGAATTCGCCCTTAAGCTAGCGCCTCGGCTCT |
| | positions 211-3440 | GGTATGAGCAAGATGGAGCTTCTGTCCTGCTTCAGTCCTGTACTG |
| | (including overlap at | TGGGATCTACTCCAGAGAGAGAAGAGCGTTTGGGCCCTGAGGAC |
| | nucleotide positions | CCTGGTGAAGGCCTACCTGCGCATGCCTCCAGAAGACCTTCAGC |
| | 211-710 with P959) | AGCTTGTGCTTTCAGCAGAGATGGAGGCTGCACAGGGCTTCCTG |
| | WPRE at nucleotide | ACGCTCATGCTTCGTTCCTGGGCTAAGCTGAAGGTTCAACCATCC |
| | positions 3452-3999 | GAGGAGCAGGCCATGGGCCGCCTGACAGCCTTGCTGCTCCAGC |
| | bGH poly(A) at nucleotide | GGTACCCACGCCTCACCTCCCAACTCTTTATCGACATGTCACCGC |
| | positions 4012-4219 | TCATCCCCTTCCTGGCTGTCCCTGACCTCATGCGCTTCCCACCGT |
| | 3′ ITR at nucleotide | CCCTTTTGGCCAACGACAGTGTCCTGGCTGCCATCAGGGATCACA |
| | positions 4307-4436 | GCTCAGGAATGAAGCCTGAACAGAAGGAGGCCCTGGCAAAACGA |
| | | CTGCTGGCCCCTGAGCTGTTTGGAGAAGTGCCTGATTGGCCCCA |
| | | GGAGCTGCTGTGGGCAGCCCTGCCTCTGCTTCCCCATCTGCCTC |
| | | TGGAGAGCTTTCTCCAGCTCAGCCCTCACCAGATCCAGGCCCTG |
| | | GAGGATAGCTGGCCAGTAGCAGATCTTGGGCCGGGACACGCCC |
| | | GACATGTGCTTCGTAGCCTAGTAAACCAGAGCATGGAGGATGGG |
| | | GAGGAGCAGGTGCTCAGGCTTGGGTCCCTCGCCTGTTTCCTGAG |
| | | TCCTGAGGAGCTACAGAGTCTGGTGCCCTTGAGTGATCCAATGG |
| | | GGCCTGTAGAACAGGGTCTGCTGGAATGTGCGGCCAATGGGACC |
| | | CTCAGCCCAGAAGGACGGGTGGCATATGAACTTCTGGGAGTGTT |
| | | GCGTTCATCTGGAGGAACTGTCTTAAGCCCCCGAGAGCTGAGGG |
| | | TCTGGGCACCTCTCTTTCCCCAGCTGGGCCTCCGCTTCCTGCAG |
| | | GAGCTCTCAGAGACCCAGCTTAGAGCCATGCTTCCTGCCCTACAG |
| | | GGAGCCAGTGTCACACCTGCCCAGGCTGTTCTGTTGTTTGGAAG |
| | | GCTCCTTCCTAAGCATGATCTGTCCCTGGAGGAACTCTGCTCCCT |
| | | GCACCCTCTCCTGCCAGGTCTCAGCCCCCAGACACTCCAGGCCA |
| | | TCCCTAAGAGAGTTCTGGTTGGTGCTTGTTCCTGCCTGGGCCCTG |
| | | AACTGTCAAGGCTTTCAGCTTGCCAGATTGCAGCTCTGCTGCAGA |
| | | CCTTTCGGGTAAAAGATGGTGTTAAAAATATGGGTGCAGCAGGTG |
| | | CCGGCTCAGCCGTGTGCATTCCTGGGCAGCCCACCACTTGGCCA |
| | | GACTGCCTGCTTCCCCTGCTCCCATTAAAGCTGCTACAGCTGGAC |
| | | GCTGCAGCTCTTCTGGCAAACCGAAGACTCTATCGGCAGCTGCCT |
| | | TGGTCTGAGCAACAGGCACAGTTTCTCTGGAAGAAAATGCAAGTG |
| | | CCTACCAACCTGAGCCTGAGGAATCTGCAGGCTCTGGGCAACTT |
| | | GGCAGGAGGCATGACCTGCGAGTTTCTGCAGCAGATCAGCTCAA |
| | | TGGTTGACTTTCTTGATGTGGTACACATGCTCTACCAGCTGCCCA |
| | | CTGGTGTTCGAGAGAGCCTGCGGGCCTGTATCTGGACAGAGCTA |
| | | CAGCGGAGGATGACAATGCCAGAGCCAGAGCTGACCACCCTAGG |
| | | GCCAGAACTGAGTGAACTTGACACAAAGCTACTCCTGGACTTGCC |
| | | GATCCAGCTGATGGACAGATTGTCCAATGATTCCATTATGTTGGT |
| | | GGTGGAGATGGTCCAAGGCGCTCCAGAGCAGCTGCTGGCACTGA |
| | | CCCCACTCCACCAGACAGCCTTGGCAGAGCGAGCACTTAAAAAC |
| | | CTGGCTCCAAAGGAGACCCCAATCTCCAAAGAAGTGCTGGAGAC |
| | | ACTGGGCCCCTTGGTTGGATTCCTGGGAATAGAGAGCACGCGAC |
| | | GGATCCCTTTACCCATTCTACTGTCTCATCTCAGTCAGCTGCAGG |
| | | GCTTCTGCCTAGGAGAGACATTTGCCACAGAGCTGGGATGGCTG |
| | | CTGTTGCAGGAGCCTGTTCTTGGAAAACCAGAATTGTGGAGCCAG |
| | | GATGAAATAGAGCAAGCTGGACGCCTAGTATTCACTCTGTCTGCT |
| | | GAGGCTATTTCCTCGATCCCCAGGGAGGCTTTGGGCCCAGAGAC |
| | | ACTGGAGAGGCTTCTGGGAAAGCATCAAAGCTGGGAGCAGAGCA |
| | | GAGTGGGCCATCTGTGTGGGGAGTCACAGCTTGCCCACAAGAAA |
| | | GCAGCTCTGGTAGCTGGGATTGTGCATCCAGCTGCTGAGGGTCT |
| | | CCAAGAGCCTGTACCAAACTGTGCAGACATACGGGGAACCTTCC |
| | | CAGCGGCCTGGTCTGCGACACAAATCTCAGAGATGGAACTCTCA |
| | | GACTTTGAAGACTGCCTGTCACTATTTGCTGGAGATCCAGGACTT |
| | | GGTCCTGAGGAACTACGGGCAGCCATGGGCAAGGCCAAGCAGTT |
| | | GTGGGGTCCCCCTCGAGGATTCCGTCCTGAGCAGATCTTGCAGC |
| | | TGGGCCGTCTCCTGATAGGTCTAGGAGAACGGGAACTGCAGGAG |
| | | CTTACCTTGGTGGACTGGGGTGTGCTGAGCAGCCTGGGGCAAAT |
| | | AGATGGCTGGAGTTCCATGCAGCTCCGAGCCGTGGTCTCCAGTT |
| | | TCCTAAGGCAGAGTGGTCGGCATGTGAGCCACCTGGACTTCATTT |
| | | ATCTGACAGCACTGGGTTACACAGTCTGTGGATTGCGACCAGAG |
| | | GAGTTACAGCACATCAGCAGTTGGGAGTTTAGCCAAGCAGCTCTC |
| | | TTCCTGGGTAGCTTGCATCTCCCGTGCTCTGAGGAACAGCTGGAA |
| | | GTTCTGGCCTATCTCCTTGTGTTGCCTGGTGGCTTTGGCCCAGTC |
| | | AGTAACTGGGGGCCTGAGATCTTCACTGAAATTGGCACAATAGCA |
| | | GCTGGCATCCCAGACCTGGCTCTTTCAGCATTACTGCGGGGACA |
| | | GATCCAAGGCCTGACTCCTCTTGCCATTTCTGTCATTCCTGCTCC |
| | | CAAGTTTGCAGTGGTCTTCAACCCCATCCAGTTATCTAGTCTCACC |
| | | AGGGGTCAGGCCGTAGCTGTTACTCCTGAACAGCTGGCCTATCT |
| | | GAGTCCTGAGCAGCGGCGAGCAGTTGCATGGGCCCAACACGAAG |
| | | GGAAGGAGATCCCAGAGCAGCTGGGTCGAAACTCAGCCTGGGGT |
| | | CTCTACGACTGGTTCCAAGCCTCCTGGGCCCTGGCATTGCCCGT |
| | | CAGCATTTTTGGCCACCTATTATGATAATAAGCTTGGATCCAATCA |
| | | ACCTCTGGATTACAAAATTTGTGAAAGATTGACTGGTATTCTTAAC |
| | | TATGTTGCTCCTTTTACGCTATGTGGATACGCTGCTTTAATGCCTT |
| | | TGTATCATGCTATTGCTTCCCGTATGGCTTTCATTTTCTCCTCCTT |
| | | GTATAAATCCTGGTTGCTGTCTCTTTATGAGGAGTTGTGGCCCGT |
| | | TGTCAGGCAACGTGGCGTGGTGTGCACTGTGTTTGCTGACGCAA |
| | | CCCCCACTGGTTGGGGCATTGCCACCACCTGTCAGCTCCTTTCC |
| | | GGGACTTTCGCTTTCCCCCTCCCTATTGCCACGGCGGAACTCATC |
| | | GCCGCCTGCCTTGCCCGCTGCTGGACAGGGGCTCGGCTGTTGG |
| | | GCACTGACAATTCCGTGGTGTTGTCGGGGAAATCATCGTCCTTTC |
| | | CTTGGCTGCTCGCCTGTGTTGCCACCTGGATTCTGCGCGGGACG |
| | | TCCTTCTGCTACGTCCCTTCGGCCCTCAATCCAGCGGACCTTCCT |
| | | TCCCGCGGCCTGCTGCCGGCTCTGCGGCCTCTTCCGCGTCTTCG |
| | | AGATCTGCCTCGACTGTGCCTTCTAGTTGCCAGCCATCTGTTGTT |
| | | TGCCCCTCCCCCGTGCCTTCCTTGACCCTGGAAGGTGCCACTCC |
| | | CACTGTCCTTTCCTAATAAAATGAGGAAATTGCATCGCATTGTCTG |
| | | AGTAGGTGTCATTCTATTCTGGGGGGTGGGGTGGGGCAGGACAG |
| | | CAAGGGGGAGGATTGGGAAGACAATAGCAGGCATGCTGGGGACT |
| | | CGAGTTAAGGGCGAATTCCCGATAAGGATCTTCCTAGAGCATGGC |
| | | TACGTAGATAAGTAGCATGGGGGGTTAATCATTAACTACAAGGAA |
| | | CCCCTAGTGATGGAGTTGGCCACTCCCTCTCTGCGCGCTCGCTC |
| | | GCTCACTGAGGCCGGGCGACCAAAGGTCGCCCGACGCCCGGGC |
| | | TTTGCCCGGGGGGCCTCAGTGAGCGAGCGAGCGCGCAGCCTTAA |
| | | TTAACCTAATTCACTGGCCGTCGTTTTACAACGTCGTGACTGGGA |
| | | AAACCCTGGCGTTACCCAACTTAATCGCCTTGCAGCACATCCCCC |
| | | TTTCGCCAGCTGGCGTAATAGCGAAGAGGCCCGCACCGATCGCC |
| | | CTTCCCAACAGTTGCGCAGCCTGAATGGCGAATGGGACGCGCCC |
| | | TGTAGCGGCGCATTAAGCGCGGCGGGTGTGGTGGTTACGCGCA |
| | | GCGTGACCGCTACACTTGCCAGCGCCCTAGCGCCCGCTCCTTTC |
| | | GCTTTCTTCCCTTCCTTTCTCGCCACGTTCGCCGGCTTTCCCCGT |
| | | CAAGCTCTAAATCGGGGGCTCCCTTTAGGGTTCCGATTTAGTGCT |
| | | TTACGGCACCTCGACCCCAAAAAACTTGATTAGGGTGATGGTTCA |
| | | CGTAGTGGGCCATCGCCCTGATAGACGGTTTTTCGCCCTTTGACG |
| | | TTGGAGTCCACGTTCTTTAATAGTGGACTCTTGTTCCAAACTGGAA |
| | | CAACACTCAACCCTATCTCGGTCTATTCTTTTGATTTATAAGGGAT |
| | | TTTGCCGATTTCGGCCTATTGGTTAAAAAATGAGCTGATTTAACAA |
| | | AAATTTAACGCGAATTTTAACAAAATATTAACGCTTACAATTTAGGT |
| | | GGCACTTTTCGGGGAAATGTGCGCGGAACCCCTATTTGTTTATTT |
| | | TTCTAAATACATTCAAATATGTATCCGCTCATGAGACAATAACCCT |
| | | GATAAATGCTTCAATAATATTGAAAAAGGAAGAGTATGAGCCATAT |
| | | TCAACGGGAAACGTCGAGGCCGCGATTAAATTCCAACATGGATGC |
| | | TGATTTATATGGGTATAAATGGGCTCGCGATAATGTCGGGCAATC |
| | | AGGTGCGACAATCTATCGCTTGTATGGGAAGCCCGATGCGCCAG |
| | | AGTTGTTTCTGAAACATGGCAAAGGTAGCGTTGCCAATGATGTTA |
| | | CAGATGAGATGGTCAGACTAAACTGGCTGACGGAATTTATGCCTC |
| | | TTCCGACCATCAAGCATTTTATCCGTACTCCTGATGATGCATGGTT |
| | | ACTCACCACTGCGATCCCCGGAAAAACAGCATTCCAGGTATTAGA |
| | | AGAATATCCTGATTCAGGTGAAAATATTGTTGATGCGCTGGCAGT |
| | | GTTCCTGCGCCGGTTGCATTCGATTCCTGTTTGTAATTGTCCTTTT |
| | | AACAGCGATCGCGTATTTCGTCTTGCTCAGGCGCAATCACGAATG |
| | | AATAACGGTTTGGTTGATGCGAGTGATTTTGATGACGAGCGTAAT |
| | | GGCTGGCCTGTTGAACAAGTCTGGAAAGAAATGCATAAACTTTTG |
| | | CCATTCTCACCGGATTCAGTCGTCACTCATGGTGATTTCTCACTTG |
| | | ATAACCTTATTTTTGACGAGGGGAAATTAATAGGTTGTATTGATGT |
| | | TGGACGAGTCGGAATCGCAGACCGATACCAGGATCTTGCCATCC |
| | | TATGGAACTGCCTCGGTGAGTTTTCTCCTTCATTACAGAAACGGC |
| | | TTTTTCAAAAATATGGTATTGATAATCCTGATATGAATAAATTGCAG |
| | | TTTCATTTGATGCTCGATGAGTTTTTCTAACTGTCAGACCAAGTTT |
| | | ACTCATATATACTTTAGATTGATTTAAAACTTCATTTTTAATTTAAAA |
| | | GGATCTAGGTGAAGATCCTTTTTGATAATCTCATGACCAAAATCCC |
| | | TTAACGTGAGTTTTCGTTCCACTGAGCGTCAGACCCCGTAGAAAA |
| | | GATCAAAGGATCTTCTTGAGATCCTTTTTTTCTGCGCGTAATCTGC |
| | | TGCTTGCAAACAAAAAAACCACCGCTACCAGCGGTGGTTTGTTTG |
| | | CCGGATCAAGAGCTACCAACTCTTTTTCCGAAGGTAACTGGCTTC |
| | | AGCAGAGCGCAGATACCAAATACTGTTCTTCTAGTGTAGCCGTAG |
| | | TTAGGCCACCACTTCAAGAACTCTGTAGCACCGCCTACATACCTC |
| | | GCTCTGCTAATCCTGTTACCAGTGGCTGCTGCCAGTGGCGATAAG |
| | | TCGTGTCTTACCGGGTTGGACTCAAGACGATAGTTACCGGATAAG |
| | | GCGCAGCGGTCGGGCTGAACGGGGGGTTCGTGCACACAGCCCA |
| | | GCTTGGAGCGAACGACCTACACCGAACTGAGATACCTACAGCGT |
| | | GAGCTATGAGAAAGCGCCACGCTTCCCGAAGGGAGAAAGGCGGA |
| | | CAGGTATCCGGTAAGCGGCAGGGTCGGAACAGGAGAGCGCACG |
| | | AGGGAGCTTCCAGGGGGAAACGCCTGGTATCTTTATAGTCCTGTC |
| | | GGGTTTCGCCACCTCTGACTTGAGCGTCGATTTTTGTGATGCTCG |
| | | TCAGGGGGGCGGAGCCTATGGAAAAACGCCAGCAACGCGGCCTT |
| | | TTTACGGTTCCTGGCCTTTTGCTGGCCTTTTGCTCACATGTTCTTT |
| | | CCTGCGTTATCCCCTGATTCTGTGGATAACCGTATTACCGCCTTT |
| | | GAGTGAGCTGATACCGCTCGCCGCAGCCGAACGACCGAGCGCA |
| | | GCGAGTCAGTGAGCGAGGAAGCGGAAGAGCGCCCAATACGCAAA |
| | | CCGCCTCTCCCCGCGCGTTGGCCGATTCATTAATGCAGCTGGCA |
| | | CGACAGGTTTCCCGACTGGAAAGCGGGCAGTGAGCGCAACGCAA |
| | | TTAATGTGAGTTAGCTCACTCATTAGGCACCCCAGGCTTTACACTT |
| | | TATGCTTCCGGCTCGTATGTTGTGTGGAATTGTGAGCGGATAACA |
| | | ATTTCACACAGGAAACAGCTATGACCATGATTACGCCAGATTTAAT |
| | | TAAGGCCTTAATTAGG |

### Trans-Splicing Dual Vectors

A second approach for expressing large proteins in mammalian cells involves the use of trans-splicing dual vectors. In this approach, two nucleic acid vectors are used that contain distinct nucleic acid sequences, and the polynucleotide encoding the N-terminal portion of the protein of interest and the polynucleotide encoding the C-terminal portion of the protein of interest do not overlap. Instead, the first nucleic acid vector includes a splice donor sequence 3′ of the polynucleotide encoding the N-terminal portion of the protein of interest, and the second nucleic acid vector includes a splice acceptor sequence 5′ of the polynucleotide encoding the C-terminal portion of the protein of interest. When the first and second nucleic acids are present in the same cell, their ITRs can concatenate, forming a single nucleic acid structure in which the concatenated ITRs are positioned between the splice donor and splice acceptor. Trans-splicing then occurs during transcription, producing a nucleic acid molecule in which the polynucleotides encoding the N-terminal and C-terminal portions of the protein of interest are contiguous, thereby forming the full-length coding sequence.

Trans-splicing dual vectors for use in the methods and compositions described herein are designed such that approximately half of the stereocilin coding sequence is contained within each vector (e.g., each vector contains a polynucleotide that encodes approximately half of the stereocilin protein, as is discussed above). The determination of how to split the polynucleotide sequence between the two nucleic acid vectors is made based on the size of the promoter and the locations of sequence elements of interest in the polynucleotide that encodes the stereocilin protein (e.g., exons of the STRC gene). The first vector in the trans-splicing dual vector system can contain a promoter sequence 5′ of a polynucleotide encoding an N-terminal portion of a stereocilin protein. The nucleic acid vectors can optionally contain STRC UTRs (e.g., both the 5′ and 3′ STRC UTRs, e.g., full-length UTRs). One exemplary trans-splicing dual vector system for use in the compositions and methods described herein includes a first nucleic acid vector containing an OCM promoter (e.g., any one of SEQ ID NOs: 1-3) operably linked to a polynucleotide encoding an N-terminal portion of a stereocilin protein (e.g., an N-terminal portion of a human stereocilin protein, e.g., an N-terminal portion of SEQ ID NO: 4) and a splice donor sequence 3′ of the polynucleotide sequence; and a second nucleic acid vector containing a splice acceptor sequence 5′ of a polynucleotide encoding a C-terminal portion of the stereocilin protein (e.g., a C-terminal portion of human stereocilin, e.g., a C-terminal portion of SEQ ID NO: 4) and a poly(A) sequence. An alternative trans-splicing dual vector system includes a first nucleic acid vector containing an OCM promoter (e.g., any one of SEQ ID NOs: 1-3) operably linked to a polynucleotide encoding an N-terminal portion of the stereocilin protein (e.g., an N-terminal portion of a murine stereocilin protein, e.g., an N-terminal portion of SEQ ID NO: 5) and a splice donor sequence 3′ of the polynucleotide sequence; and a second nucleic acid vector containing a splice acceptor sequence 5′ of a polynucleotide encoding a C-terminal portion of the stereocilin protein (e.g., a C-terminal portion of a murine stereocilin protein, e.g., a C-terminal portion of SEQ ID NO: 5) and a poly(A) sequence. In some embodiments, the OCM promoter is a polynucleotide having the sequence of SEQ ID NO: 1 or a variant having at least 85% (e.g., at least 86%, 87%, 88%, 89%, 90%, 91%, 92%, 93%, 94%, 95%, 96%, 97%, 98%, 99%, or more) sequence identity to SEQ ID NO: 1. In some embodiments, the OCM promoter is a polynucleotide having the sequence of SEQ ID NO: 2 or a variant having at least 85% (e.g., at least 86%, 87%, 88%, 89%, 90%, 91%, 92%, 93%, 94%, 95%, 96%, 97%, 98%, 99%, or more) sequence identity to SEQ ID NO: 2. In some embodiments, the OCM promoter is a polynucleotide having the sequence of SEQ ID NO: 3 or a variant having at least 85% (e.g., at least 86%, 87%, 88%, 89%, 90%, 91%, 92%, 93%, 94%, 95%, 96%, 97%, 98%, 99%, or more) sequence identity to SEQ ID NO: 3. These nucleic acid vectors can also contain full-length 5′ and/or 3′ STRC UTRs in the first and second nucleic acid vectors, respectively (e.g., the first nucleic acid vector can contain the 5′ human STRC UTR in dual vector systems encoding human stereocilin, or the 5′ mouse UTR in dual vector systems encoding mouse stereocilin; and the second nucleic acid vector can contain the 3′ human STRC UTR in dual vector systems encoding human stereocilin, or the 3′ mouse STRC UTR in dual vector systems encoding mouse stereocilin). To accommodate an STRC UTR, the stereocilin coding sequence can be divided at such a position as to accommodate the length of the promoter sequence and the sequence encoding the N-terminal portion of stereocilin.

In some embodiments, the polynucleotide that encodes a full-length human stereocilin protein has the sequence of SEQ ID NO: 6 or is a variant thereof having at least 85% (e.g., at least 86%, 87%, 88%, 89%, 90%, 91%, 92%, 93%, 94%, 95%, 96%, 97%, 98%, 99%, or more) sequence identity to SEQ ID NO: 6. In some embodiments, the polynucleotide that encodes a full-length murine stereocilin protein has the sequence of SEQ ID NO: 7 or is a variant thereof having at least 85% (e.g., at least 86%, 87%, 88%, 89%, 90%, 91%, 92%, 93%, 94%, 95%, 96%, 97%, 98%, 99%, or more) sequence identity SEQ ID NO: 7.

### Dual Hybrid Vectors

A third approach for expressing large proteins in mammalian cells involves the use of dual hybrid vectors. This approach combines elements of the overlapping dual vector strategy and the trans-splicing strategy in that it features both an overlapping region at which homologous recombination can occur and splice donor and splice acceptor sequences. In dual hybrid vector systems, the overlapping region is a recombinogenic region that is contained in both the first and second nucleic acid vectors, rather than a portion of the polynucleotide sequence encoding the protein of interest—the polynucleotide encoding the N-terminal portion of the protein of interest and the polynucleotide encoding the C-terminal portion of the protein of interest do not overlap in this approach. The recombinogenic region is 3′ of the splice donor sequence in the first nucleic acid vector and 5′ of the splice acceptor sequence in the second nucleic acid sequence. The first and second nucleic acid sequences can then join to form a single sequence based on one of two mechanisms: 1) recombination at the overlapping region, or 2) concatemerization of the ITRs. The remaining recombinogenic region(s) and/or the concatemerized ITRs can be removed by splicing, leading to the formation of a contiguous polynucleotide sequence that encodes the full-length protein of interest. Recombinogenic regions, splice donor sequences, and splice acceptor sequences that can be used in the compositions and methods described herein include those well-known to one of skill in the art. Exemplary recombinogenic regions include the F1 phage AK gene and alkaline phosphatase (AP) gene fragments as described in U.S. Pat. Nos. 10,494,645 and 8,236,557, which are incorporated herein by reference. In some embodiments, the AP gene fragment has the sequence of:

(SEQ ID NO: 47)
CCCCGGGTGCGCGGCGTCGGTGGTGCCGGGGGGGGCGCCAGGTCGCAGG
CGGTGTAGGGCTCCAGGCAGGCGGCGAAGGCCATGACGTGCGCTATGAA
GGTCTGCTCCTGCACGCCGTGAACCAGGTGCGCCTGCGGGCCGCGCGCG
AACACCGCCACGTCCTCGCCTGCGTGGGTCTCTTCGTCCAGGGGCACTG
CTGACTGCTGCCGATACTCGGGGCTCCCGCTCTCGCTCTCGGTAACATC
CGGCCGGGCGCCGTCCTTGAGCACATAGCCTGGACCGTTTCCGTATAGG
AGGACCGTGTAGGCCTTCCTGTCCCGGGCCTTGCCAGCGGCCAGCCCGA
TGAAGGAGCTCCCTCGCAGGGGGTAGCCTCCGAAGGAGAAGACGTGGGA
GTGGTCGGCAGTGACGAGGCTCAGCGTGTCCTCCTCGCTGGTGAGCTGG
CCCGCCCTCTCAATGGCGTCGTCGAACATGATCGTCTCAGTCAGTGCCC
GGTAAGCCCTGCTTTCATGATGACCATGGTCGATGCGACCACCCTCCAC
GAAGAGGAAGAAGCCGCGGGGGTGTCTGCTCAGCAGGCGCAGGGCAGCC
TCTGTCATCTCCATCAGGGAGGGGTCCAGTGTGGAGTCTCGGTGGATCT
CGTATTTCATGTCTCCAGGCTCAAAGAGACCCATGAGATGGGTCACAGA
CGGGTCCAGGGAAGCCTGCATGAGCTCAGTGCGGTTCCACACGTACCGG
GCACCCTGGCGTTCGCCGAGCCATTCCTGCACCAGATTCTTCCCGTCCA
GCCTGGTCCCACCTTGGCTGTAGTCATCTGGGTACTCAGGGTCTGGGGT
TCCCATGCGAAACATGTACTTTCGGCCTCCA.

In some embodiments, the AP gene fragment has the sequence of:

(SEQ ID NO: 48)
CCCCGGGTGCGCGGCGTCGGTGGTGCCGGGGGGGGCGCCAGGTCGCAGG
CGGTGTAGGGCTCCAGGCAGGCGGCGAAGGCCATGACGTGCGCTATGAA
GGTCTGCTCCTGCACGCCGTGAACCAGGTGCGCCTGCGGGCCGCGCGCG
AACACCGCCACGTCCTCGCCTGCGTGGGTCTCTTCGTCCAGGGGCACTG
CTGACTGCTGCCGATACTCGGGGCTCCCGCTCTCGCTCTCGGTAACATC
CGGCCGGGCGCCGTCCTTGAGCACATAGCCTGGACCGTTTCCGTATAGG
AGGACCGTGTAGGCCTTCCTGTCCCGGGCCTTGCCAGCGGCCAGCCCGA
TGAAGGAGCTCCCTCGCAGGGGGTAGCCTCCGAAGGAGAAGACGTGGGA
GTGGTCGGCAGTGACGAGGCTCAGCGTGTCCTCCTCG CTGGTGA.

In some embodiments, the AP gene fragment has the sequence of:

(SEQ ID NO: 49)
GCTGGCCCGCCCTCTCAATGGCGTCGTCGAACATGATCGTCTCAGTCAG
TGCCCGGTAAGCCCTGCTTTCATGATGACCATGGTCGATGCGACCACCC
TCCACGAAGAGGAAGAAGCCGCGGGGGTGTCTGCTCAGCAGGCGCAGGG
CAGCCTCTGTCATCTCCATCAGGGAGGGGTCCAGTGTGGAGTCTCGGTG
GATCTCGTATTTCATGTCTCCAGGCTCAAAGAGACCCATGAGATGGGTC
ACAGACGGGTCCAGGGAAGCCTGCATGAGCTCAGTGCGGTTCCACACGT
ACCGGGCACCCTGGCGTTCGCCGAGCCATTCCTGCACCAGATTCTTCCC
GTCCAGCCTGGTCCCACCTTGGCTGTAGTCATCTGGGTACTCAGGGTCT
GGGGTTCCCATGCGAAACATGTACTTTCGGCCTCCA.

In some embodiments, the AP gene fragment has the sequence of:

(SEQ ID NO: 50)
CCCCGGGTGCGCGGCGTCGGTGGTGCCGGGGGGGGCGCCAGGTCGCAGG
CGGTGTAGGGCTCCAGGCAGGCGGCGAAGGCCATGACGTGCGCTATGAA
GGTCTGCTCCTGCACGCCGTGAACCAGGTGCGCCTGCGGGCCGCGCGCG
AACACCGCCACGTCCTCGCCTGCGTGGGTCTCTTCGTCCAGGGGCACTG
CTGACTGCTGCCGATACTCGGGGCTCCCGCTCTCGCTCTCGGTAACATC
CGGCCGGGCGCCGTCCTTGAGCACATAGCCTGGACCGTTTC

In some embodiments, the AP gene fragment has the sequence of:

(SEQ ID NO: 51)
CGTATAGGAGGACCGTGTAGGCCTTCCTGTCCCGGGCCTTGCCAGCGGC
CAGCCCGATGAAGGAGCTCCCTCGCAGGGGGTAGCCTCCGAAGGAGAAG
ACGTGGGAGTGGTCGGCAGTGACGAGGCTCAGCGTGTCCTCCTCGCTGG
TGAGCTGGCCCGCCCTCTCAATGGCGTCGTCGAACATGATCGTCTCAGT
CAGTGCCCGGTAAGCCCTGCTTTCATGATGACCATGGTCGATGCGACCA
CCCTCCACGAAGAGGAAGAAGCCGCGGGGGTGTCTGCTCAGCAGG.

In some embodiments, the AP gene fragment has the sequence of:

(SEQ ID NO: 52)
CGCAGGGCAGCCTCTGTCATCTCCATCAGGGAGGGGTCCAGTGTGGAGT
CTCGGTGGATCTCGTATTTCATGTCTCCAGGCTCAAAGAGACCCATGAG
ATGGGTCACAGACGGGTCCAGGGAAGCCTGCATGAGCTCAGTGCGGTTC
CACACGTACCGGGCACCCTGGCGTTCGCCGAGCCATTCCTGCACCAGAT
TCTTCCCGTCCAGCCTGGTCCCACCTTGGCTGTAGTCATCTGGGTACTC
AGGGTCTGGGGTTCCCATGCGAAACATGTACTTTCGGCCTCCA.

Dual hybrid vectors for use in the methods and compositions described herein are designed such that approximately half of the stereocilin coding sequence is contained within each vector (e.g., each vector contains a polynucleotide that encodes approximately half of the stereocilin protein). The determination of how to split the polynucleotide sequence between the two nucleic acid vectors is made based on the size of the promoter and the locations of sequence elements of interest in the polynucleotide that encodes the stereocilin protein (e.g., exons of the STRC gene). The first vector in the trans-splicing dual vector system can contain a promoter sequence 5′ of a polynucleotide encoding an N-terminal portion of a stereocilin protein. The nucleic acid vectors can optionally contain STRC UTRs (e.g., full-length 5′ and 3′ UTRs).

One exemplary dual hybrid vector system includes a first nucleic acid vector containing an OCM promoter (e.g., any one of SEQ ID NOs: 1-3) operably linked to a polynucleotide encoding an N-terminal portion of a stereocilin protein (e.g., an N-terminal portion of human stereocilin, e.g., an N-terminal portion of SEQ ID NO: 4), a splice donor sequence 3′ of the polynucleotide sequence, and a recombinogenic region 3′ of the splice donor sequence; and a second nucleic acid vector containing a recombinogenic region (e.g., the same recombinogenic region that is included in the first vector), a splice acceptor sequence 3′ of the recombinogenic region, a polynucleotide 3′ of the splice acceptor sequence that encodes a C-terminal portion of the stereocilin protein (e.g., a C-terminal portion of human stereocilin, e.g., a C-terminal portion of SEQ ID NO: 4), and a poly(A) sequence. In some embodiments, the OCM promoter is a polynucleotide having the sequence of SEQ ID NO: 1 or a variant having at least 85% (e.g., at least 86%, 87%, 88%, 89%, 90%, 91%, 92%, 93%, 94%, 95%, 96%, 97%, 98%, 99%, or more) sequence identity to SEQ ID NO: 1. In some embodiments, the OCM promoter is a polynucleotide having the sequence of SEQ ID NO: 2 or a variant having at least 85% (e.g., at least 86%, 87%, 88%, 89%, 90%, 91%, 92%, 93%, 94%, 95%, 96%, 97%, 98%, 99%, or more) sequence identity to SEQ ID NO: 2. In some embodiments, the OCM promoter is a polynucleotide having the sequence of SEQ ID NO: 3 or a variant having at least 85% (e.g., at least 86%, 87%, 88%, 89%, 90%, 91%, 92%, 93%, 94%, 95%, 96%, 97%, 98%, 99%, or more) sequence identity to SEQ ID NO: 3. The first and second nucleic acid vectors can also contain the full length 5′ and/or 3′ STRC UTRs, respectively (e.g., the human STRC 5′ UTR can be included in the first nucleic acid vector, and the human STRC 3′ UTR can be included in the second nucleic acid vector). Another exemplary dual hybrid vector system that includes an OCM promoter includes a first nucleic acid vector containing an OCM promoter (e.g., any one of SEQ ID NOs: 1-3) operably linked to polynucleotide encoding an N-terminal portion of a stereocilin protein (e.g., am N-terminal portion of murine stereocilin, e.g., an N-terminal portion of SEQ ID NO: 5), a splice donor sequence 3′ of the polynucleotide sequence, and a recombinogenic region 3′ of the splice donor sequence; and a second nucleic acid vector containing a recombinogenic region (e.g., the same recombinogenic region that is included in the first vector), a splice acceptor sequence 3′ of the recombinogenic region, a polynucleotide 3′ of the splice acceptor sequence that encodes a C-terminal portion of the stereocilin protein (e.g., a C-terminal portion of murine stereocilin, e.g., a C-terminal portion of SEQ ID NO: 5), and a poly(A) sequence. The first and second nucleic acid vectors can also contain the full length 5′ and/or 3′ STRC UTRs, respectively (e.g., the mouse STRC 5′ UTR can be included in the first nucleic acid vector, and the mouse STRC 3′ UTR can be included in the second nucleic acid vector). To accommodate an STRC UTR, the stereocilin coding sequence can be divided at a different position than it would be in a dual hybrid vector system that does not include an STRC UTR.

In some embodiments, the polynucleotide that encodes a full-length human stereocilin protein has the sequence of SEQ ID NO: 6 or is a variant thereof having at least 85% (e.g., at least 86%, 87%, 88%, 89%, 90%, 91%, 92%, 93%, 94%, 95%, 96%, 97%, 98%, 99%, or more) sequence identity to SEQ ID NO: 6. In some embodiments, the polynucleotide that encodes a full-length murine stereocilin protein has the sequence of SEQ ID NO: 7 or is a variant thereof having at least 85% (e.g., at least 86%, 87%, 88%, 89%, 90%, 91%, 92%, 93%, 94%, 95%, 96%, 97%, 98%, 99%, or more) sequence identity SEQ ID NO: 7.

The dual hybrid vectors used in the methods and compositions described herein can optionally include a degradation signal sequence in both the first and second nucleic acid vectors. The degradation signal sequence can be included to prevent or reduce the expression of portions of the stereocilin protein from polynucleotides that failed to recombine and/or undergo splicing. The degradation signal sequence is positioned 3′ of the recombinogenic region in the first nucleic acid vector, and is positioned between the recombinogenic region and the splice acceptor in the second nucleic acid vector. Suitable degradation signal sequences that can be used in the compositions and methods described herein are known in the art and are described, for example, in International Application Publication No. WO 2016/139321, which is incorporated herein by reference.

In some embodiments, the first member of the dual vector system includes the OCM promoter of SEQ ID NO:1 (also represented by nucleotides 225-1364 of SEQ ID NO: 45) operably linked to nucleotides that encode an N-terminal portion of a stereocilin protein. In certain embodiments, the nucleotide sequence that encodes an N-terminal portion of a stereocilin protein is nucleotides 1378-4077 of SEQ ID NO: 45. The nucleotide sequences that encode an N-terminal portion of a stereocilin protein can be partially or fully codon-optimized for expression. In some embodiments, the first member of the dual vector system includes the splice donor sequence corresponding to nucleotides 4078-4161 of SEQ ID NO: 45. In some embodiments, the first member of the dual vector system includes the AP head sequence corresponding to nucleotides 4168-4454 of SEQ ID NO: 45. In particular embodiments, the first member of the dual vector system includes nucleotides 225-4454 of SEQ ID NO: 45 flanked on each of the 5′ and 3′ sides by an inverted terminal repeat. In some embodiments, the flanking inverted terminal repeats are any variant of AAV2 inverted terminal repeats that can be encapsidated by a plasmid that carries the AAV2 Rep gene. In certain embodiments, the 5′ flanking inverted terminal repeat has a sequence corresponding to nucleotides 1-130 of SEQ ID NO: 45 or a sequence having at least 80% sequence identity (at least 80%, 81%, 82%, 83%, 84%, 85%, 86%, 87%, 88%, 89%, 90%, 91%, 92%, 93%, 94%, 95%, 96%, 97%, 98%, or 99% sequence identity) thereto; and the 3′ flanking inverted terminal repeat has a sequence corresponding to nucleotides 4548-4677 of SEQ ID NO: 45 or a sequence having at least 80% sequence identity (at least 80%, 81%, 82%, 83%, 84%, 85%, 86%, 87%, 88%, 89%, 90%, 91%, 92%, 93%, 94%, 95%, 96%, 97%, 98%, or 99% sequence identity) thereto. It will be understood by those of skill in the art that, for any given pair of inverted terminal repeat sequences in a transfer plasmid that is used to create the viral vector (typically by transfecting cells with that plasmid together with other plasmids carrying the necessary AAV genes for viral vector formation) (e.g., SEQ ID NO: 45), that the corresponding sequence in the viral vector can be altered due to the ITRs adopting a “flip” or “flop” orientation during recombination. Thus, the sequence of the ITR in the transfer plasmid is not necessarily the same sequence that is found in the viral vector prepared therefrom. However, in some very specific embodiments, the first member of the dual vector system includes nucleotides 1-4677 of SEQ ID NO: 45.

In some embodiments, the second member of the dual vector system includes nucleotides that encode the C-terminal portion of the stereocilin protein immediately followed by a stop codon. In certain embodiments, the nucleotide sequence that encodes the C-terminal portion of the stereocilin protein is nucleotides 615-3344 of SEQ ID NO: 46. The nucleotide sequences that encode the C-terminal portion of the stereocilin protein can be partially or fully codon-optimized for expression. In some embodiments, the second member of the dual vector system includes the splice acceptor sequence corresponding to nucleotides 566-614 of SEQ ID NO: 46. In some embodiments, the second member of the dual vector system includes the AP head sequence corresponding to nucleotides 257-543 of SEQ ID NO: 46. In some embodiments, the second member of the dual vector system includes the poly(A) sequence corresponding to nucleotides 3376-3597 of SEQ ID NO: 46. In particular embodiments, the second member of the dual vector system includes nucleotides 257-3597 of SEQ ID NO: 46 flanked on each of the 5′ and 3′ sides by an inverted terminal repeat. In some embodiments, the flanking inverted terminal repeats are any variant of AAV2 inverted terminal repeats that can be encapsidated by a plasmid that carries the AAV2 Rep gene. In certain embodiments, the 5′ flanking inverted terminal repeat has a sequence corresponding to nucleotides 12-141 of SEQ ID NO: 46 or a sequence having at least 80% sequence identity (at least 80%, 81%, 82%, 83%, 84%, 85%, 86%, 87%, 88%, 89%, 90%, 91%, 92%, 93%, 94%, 95%, 96%, 97%, 98%, or 99% sequence identity) thereto; and the 3′ flanking inverted terminal repeat has a sequence corresponding to nucleotides 3685-3814 of SEQ ID NO: 46 or a sequence having at least 80% sequence identity (at least 80%, 81%, 82%, 83%, 84%, 85%, 86%, 87%, 88%, 89%, 90%, 91%, 92%, 93%, 94%, 95%, 96%, 97%, 98%, or 99% sequence identity) thereto. It will be understood by those of skill in the art that, for any given pair of inverted terminal repeat sequences in a transfer plasmid that is used to create the viral vector (typically by transfecting cells with that plasmid together with other plasmids carrying the necessary AAV genes for viral vector formation) (e.g., SEQ ID NO: 46), that the corresponding sequence in the viral vector can be altered due to the ITRs adopting a “flip” or “flop” orientation during recombination. Thus, the sequence of the ITR in the transfer plasmid is not necessarily the same sequence that is found in the viral vector prepared therefrom. However, in some very specific embodiments, the first member of the dual vector system includes nucleotides 12-3814 of SEQ ID NO: 46.

Transfer plasmids that may be used to produce nucleic acid vectors (e.g., AAV vectors) for co-formulation or co-administration (e.g., administration simultaneously or sequentially) in a dual hybrid vector system are provided in Table 5 (SEQ ID NO: 45 and SEQ ID NO: 46).

TABLE 5
Transfer plasmids designed to produce dual hybrid vectors

| SEQ | | |
|---|---|---|
| ID | | |
| NO. | Description | Plasmid Sequence |
| 45 | Plasmid P960 | CTGCGCGCTCGCTCGCTCACTGAGGCCGCCCGGGCAAAGCCCG |
| | 5′ ITR at nucleotide | GGCGTCGGGCGACCTTTGGTCGCCCGGCCTCAGTGAGCGAGCG |
| | positions 1-130 | AGCGCGCAGAGAGGGAGTGGCCAACTCCATCACTAGGGGTTCCT |
| | Murine OCM promoter at | TGTAGTTAATGATTAACCCGCCATGCTACTTATCTACGTAGCCATG |
| | nucleotide positions | CTCTAGGAAGATCGGAATTCGCCCTTAAGCTAGCGGCGCGCCAC |
| | 225-1364 | CGGTAGCAGGTTTGTTACAGAAACCTTAGTTAAGGTTTGTTGAGG |
| | N-terminal STRC coding | GTTTTTTTTCTCTCTCTCTCTCTTAATTGGCTGTCCCAATCCATCCT |
| | sequence at nucleotide | TCTATAAATAGAAAAGAGAGACAGGGAGTGTGTGTGGTTTCATTA |
| | positions 1378-4077 | CTAAGGTAAAGACACTTGAGCTACACACACTTGATCCCTGAACAT |
| | AP splice donor at | GAAATCTAAGAGGTTGAACGATCACAGTTTCAGGACTATATAAGG |
| | nucleotide positions | TGGTGAAAGACCATCTGCTTCGTTTTTCTGTTTGTTCCTACAACTC |
| | 4078-4161 | TTTCCCTCCGCTTGATTTTAACTCTAAATTGGTGAGTAGCTGGTGG |
| | AP head sequence at | GCTCACCAGACTCCGAGATCCTCTTCTCTGCACGCACTGTATTAG |
| | nucleotide positions | ACTTGGCACCCGGGAGGATTTTCACCTCTGCTGCATGGGCTAATC |
| | 4168-4454 | TTCCACAAGGGATCTGTGGTATTGCAATCTCGGGTTGATGCATGA |
| | 3′ ITR at nucleotide | CGGTGATGTTGTGTTTATAGCATGGCTAAGGTTTAGCTGCCTATG |
| | positions 4548-4677 | ATGATTGGTTAGGGAAGGATAATTTTTGCTAGAAGATTGGACTTTA |
| | | GGGAAAAAAAACCCCACTTTTATTTGCTTTTAGAATTTTAAAAGACT |
| | | GGGCCATGTAGCTCAGGCTGGTTTGGAGTTCATTATGTAGTCAAG |
| | | GATGCTCTTGGACTCTTTAGCATCCTCCTCCTCCTCTTTTTCCTCC |
| | | TCCTCCTTCTTGTTCTTCTTCTTGTTCTTCCTCTTCCCCTTCTCTTC |
| | | CCCCTTCTCTCCCTCTTCCTCCTCTTCCTCCTCCTTCTTGTTCTTC |
| | | TTCCTCTTCCCCTTCTCCTCTCCCCCTTGTCCTCCTCTTCCTTCTC |
| | | CTCCTCCTCCTCTTCTTCTTTCTGAGTACCAAGATTGCAAGTGTGC |
| | | ACACGATGACCAGCTTGGTCTTTCTTTGTCTTTTTTTTTTAACTTCA |
| | | ATTTTGGAGTGAATTCAAGAGCAACCATGTAGTCAAGAGGTGGCT |
| | | GGAGTCTTTTCTGTATCTGGGTTTGGTTTAGTACTCTGCCCCATCA |
| | | CTTAACAGGTCCTTATGGCCACATCTTAAAAAAATTCTAGAGATAC |
| | | ACGGTGTCGGTGAGTGGCTGAGAATGTGTGGTCTTCCCATTTCTC |
| | | TGTCACCGTGGCTCACATCTTGTTTCCTCTGTTCGGCCAGGTAGA |
| | | AAGGCGGCCGCCACCATGGCTCTGAGCCTCCAGCCCCAGCTGCT |
| | | CCTTCTCCTGTCGCTCCTGCCGCAGGAAGTGACTTCAGCCCCTAC |
| | | TGGGCCTCAGTCTTTGGATGCTGGTCTCTCCCTTCTGAAGTCATT |
| | | CGTAGCCACTCTGGACCAAGCTCCTCAGCGTTCCCTCAGCCAGT |
| | | CACGGTTCTCTGCGTTCCTGGCCAACATTTCTTCATCCTTCCAGCT |
| | | TGGGAGGATGGGGGAGGGACCGGTGGGAGAGCCCCCACCTCTC |
| | | CAGCCCCCTGCACTTCGACTTCATGATTTCCTCGTGACACTGAGA |
| | | GGTAGCCCAGACTGGGAGCCAATGCTAGGGCTTCTGGGAGATGT |
| | | GCTGGCACTCCTGGGACAGGAACAGACTCCCCGGGACTTTTTGG |
| | | TGCACCAGGCAGGTGTACTGGGTGGACTTGTAGAGGCATTGTTG |
| | | GGAGCGTTAGTTCCTGGAGGCCCCCCTGCCCCCACTCGACCCCC |
| | | ATGCACCCGTGATGGCCCTTCTGACTGTGTCCTGGCTGCTGATTG |
| | | GTTGCCTTCTCTGATGTTGTTATTAGAGGGTACACGCTGGCAGGC |
| | | CCTGGTGCAGTTGCAGCCCAGTGTGGACCCAACCAATGCCACAG |
| | | GTCTTGATGGTAGAGAGCCAGCTCCTCACTTTTTACAGGGTCTGC |
| | | TGGGCTTGCTTACCCCAGCAGGAGAGTTGGGCTCTGAGGAGGCT |
| | | CTTTGGGGTGGTCTGCTGCGCACAGTGGGGGCCCCCCTCTATGC |
| | | TGCCTTCCAGGAGGGGCTACTGCGAGTCACTCATTCTCTGCAAGA |
| | | TGAGGTCTTTTCTATTATGGGACAGCCAGAGCCTGATGCCAGTGG |
| | | GCAGTGCCAGGGAGGCAACCTTCAACAGCTGCTTTTATGGGGCA |
| | | TGCGGAACAACCTTTCTTGGGACGCCCGAGCACTGGGTTTTCTAT |
| | | CTGGATCACCACCTCCACCCCCTGCTCTCCTGCACTGCCTGAGCA |
| | | GAGGTGTGCCTCTGCCCAGGGCTTCCCAGCCTGCGGCTCACATC |
| | | AGCCCTCGACAGCGGCGAGCCATCTCTGTGGAGGCCCTCTGCGA |
| | | GAACCACTCAGGCCCAGAGCCACCCTACAGCATCTCCAACTTCTC |
| | | CATCTACTTGCTCTGCCAGCACATCAAGCCTGCCACCCCGCGGC |
| | | CCCCTCCTACCACCCCACGGCCTCCTCCTACCACCCCACAGCCC |
| | | CCTCCTACCACTACACAGCCCATTCCTGACACTACACAGCCCCCT |
| | | CCTGTCACCCCAAGGCCTCCTCCTACCACCCCACAACCCCCTCCT |
| | | AGCACAGCTGTCATCTGCCAGACAGCTGTATGGTACGCAGTCTCG |
| | | TGGGCACCAGGTGCCCGAGGTTGGCTCCAAGCCTGCCATGATCA |
| | | GTTTCCTGATCAATTTCTGGATATGATCTGCGGCAACCTCTCATTT |
| | | TCAGCCCTGTCTGGCCCCAGTCGTCCTTTGGTAAAGCAGCTCTGT |
| | | GCTGGCTTGCTCCCACCCCCCACTAGCTGTCCACCAGGCCTGAT |
| | | CCCTGTGCCCCTCACCCCAGAAATATTCTGGGGCTGTTTCCTGGA |
| | | GAATGAGACACTGTGGGCTGAACGGTTGTGTGTGGAGGACAGTC |
| | | TGCAGGCTGTGCCCCCGAGGAACCAGGCTTGGGTTCAGCATGTG |
| | | TGTCGGGGCCCCACCTTGGACGCCACTGATTTTCCACCGTGCCG |
| | | CGTTGGACCCTGTGGGGAACGCTGCCCAGATGGGGGCAGCTTCC |
| | | TGCTCATGGTCTGTGCCAATGACACTCTGTATGAAGCCTTGGTTC |
| | | CCTTCTGGGCTTGGCTAGCAGGCCAATGCAGAATTAGTCGTGGA |
| | | GGAAATGATACTTGCTTTCTAGAAGGCATGCTGGGCCCCTTGTTG |
| | | CCCTCTCTGCCCCCTCTGGGACCATCCCCACTCTGTCTGGCTCCT |
| | | GGTCCTTTTCTGCTTGGCATGTTATCCCAGTTGCCACGCTGTCAG |
| | | TCCTCCGTGCCAGCCCTCGCCCACCCCACGCGCCTACATTACCT |
| | | CCTGCGCCTACTGACCTTCCTTCTGGGTCCAGGGACTGGGGGTG |
| | | CCGAGACGCAGGGGATGTTAGGTCAAGCCCTGCTGCTCTCTAGT |
| | | CTCCCAGACAACTGTTCATTCTGGGATGCCTTCCGCCCAGAGGG |
| | | CCGGAGAAGTGTACTGAGGACAGTCGGAGAGTACTTGCAGCGGG |
| | | AAGAGCCAACCCCACCAGGCTTAGACTCCTCCCTCAGCCTCGGC |
| | | TCTGGTATGAGCAAGATGGAGCTTCTGTCCTGCTTCAGTCCTGTA |
| | | CTGTGGGATCTACTCCAGAGAGAGAAGAGCGTTTGGGCCCTGAG |
| | | GACCCTGGTGAAGGCCTACCTGCGCATGCCTCCAGAAGACCTTC |
| | | AGCAGCTTGTGCTTTCAGCAGAGATGGAGGCTGCACAGGGCTTC |
| | | CTGACGCTCATGCTTCGTTCCTGGGCTAAGCTGAAGGTTCAACCA |
| | | TCCGAGGAGCAGGCCATGGGCCGCCTGACAGCCTTGCTGCTCCA |
| | | GCGGTACCCACGCCTCACCTCCCAACTCTTTATCGACATGTCACC |
| | | GCTCATCCCCTTCCTGGCTGTCCCTGACCTCATGCGCTTCCCACC |
| | | GTCCCTTTTGGCCAACGACAGTGTCCTGGCTGCCATCAGGGATCA |
| | | CAGCTCAGGAATGAAGCCTGAACAGAAGGAGGCCCTGGCAAAAC |
| | | GACTGCTGGCCCCTGAGCTGTTTGGAGAAGTGCCTGATTGGCCC |
| | | CAGGTAAGTATCAAGGTTACAAGACAGGTTTAAGGAGACCAATAG |
| | | AAACTGGGCTTGTCGAGACAGAGAAGACTCTTGCGTTTCTGAGCT |
| | | AGCCCCCGGGTGCGCGGCGTCGGTGGTGCCGGGGGGGGCGC |
| | | CAGGTCGCAGGCGGTGTAGGGCTCCAGGCAGGCGGCGAAGGCC |
| | | ATGACGTGCGCTATGAAGGTCTGCTCCTGCACGCCGTGAACCAG |
| | | GTGCGCCTGCGGGCCGCGCGCGAACACCGCCACGTCCTCGCCT |
| | | GCGTGGGTCTCTTCGTCCAGGGGCACTGCTGACTGCTGCCGATA |
| | | CTCGGGGCTCCCGCTCTCGCTCTCGGTAACATCCGGCCGGGCGC |
| | | CGTCCTTGAGCACATAGCCTGGACCGTTTCGTCGACCTCGAGTTA |
| | | AGGGCGAATTCCCGATAAGGATCTTCCTAGAGCATGGCTACGTAG |
| | | ATAAGTAGCATGGGGGGTTAATCATTAACTACAAGGAACCCCTAG |
| | | TGATGGAGTTGGCCACTCCCTCTCTGCGCGCTCGCTCGCTCACT |
| | | GAGGCCGGGCGACCAAAGGTCGCCCGACGCCCGGGCTTTGCCC |
| | | GGGCGGCCTCAGTGAGCGAGCGAGCGCGCAGCCTTAATTAACCT |
| | | AATTCACTGGCCGTCGTTTTACAACGTCGTGACTGGGAAAACCCT |
| | | GGCGTTACCCAACTTAATCGCCTTGCAGCACATCCCCCTTTCGCC |
| | | AGCTGGCGTAATAGCGAAGAGGCCCGCACCGATCGCCCTTCCCA |
| | | ACAGTTGCGCAGCCTGAATGGCGAATGGGACGCGCCCTGTAGCG |
| | | GCGCATTAAGCGCGGCGGGTGTGGTGGTTACGCGCAGCGTGAC |
| | | CGCTACACTTGCCAGCGCCCTAGCGCCCGCTCCTTTCGCTTTCTT |
| | | CCCTTCCTTTCTCGCCACGTTCGCCGGCTTTCCCCGTCAAGCTCT |
| | | AAATCGGGGGCTCCCTTTAGGGTTCCGATTTAGTGCTTTACGGCA |
| | | CCTCGACCCCAAAAAACTTGATTAGGGTGATGGTTCACGTAGTGG |
| | | GCCATCGCCCTGATAGACGGTTTTTCGCCCTTTGACGTTGGAGTC |
| | | CACGTTCTTTAATAGTGGACTCTTGTTCCAAACTGGAACAACACTC |
| | | AACCCTATCTCGGTCTATTCTTTTGATTTATAAGGGATTTTGCCGA |
| | | TTTCGGCCTATTGGTTAAAAAATGAGCTGATTTAACAAAAATTTAA |
| | | CGCGAATTTTAACAAAATATTAACGCTTACAATTTAGGTGGCACTT |
| | | TTCGGGGAAATGTGCGCGGAACCCCTATTTGTTTATTTTTCTAAAT |
| | | ACATTCAAATATGTATCCGCTCATGAGACAATAACCCTGATAAATG |
| | | CTTCAATAATATTGAAAAAGGAAGAGTATGAGCCATATTCAACGGG |
| | | AAACGTCGAGGCCGCGATTAAATTCCAACATGGATGCTGATTTAT |
| | | ATGGGTATAAATGGGCTCGCGATAATGTCGGGCAATCAGGTGCG |
| | | ACAATCTATCGCTTGTATGGGAAGCCCGATGCGCCAGAGTTGTTT |
| | | CTGAAACATGGCAAAGGTAGCGTTGCCAATGATGTTACAGATGAG |
| | | ATGGTCAGACTAAACTGGCTGACGGAATTTATGCCTCTTCCGACC |
| | | ATCAAGCATTTTATCCGTACTCCTGATGATGCATGGTTACTCACCA |
| | | CTGCGATCCCCGGAAAAACAGCATTCCAGGTATTAGAAGAATATC |
| | | CTGATTCAGGTGAAAATATTGTTGATGCGCTGGCAGTGTTCCTGC |
| | | GCCGGTTGCATTCGATTCCTGTTTGTAATTGTCCTTTTAACAGCGA |
| | | TCGCGTATTTCGTCTTGCTCAGGCGCAATCACGAATGAATAACGG |
| | | TTTGGTTGATGCGAGTGATTTTGATGACGAGCGTAATGGCTGGCC |
| | | TGTTGAACAAGTCTGGAAAGAAATGCATAAACTTTTGCCATTCTCA |
| | | CCGGATTCAGTCGTCACTCATGGTGATTTCTCACTTGATAACCTTA |
| | | TTTTTGACGAGGGGAAATTAATAGGTTGTATTGATGTTGGACGAGT |
| | | CGGAATCGCAGACCGATACCAGGATCTTGCCATCCTATGGAACTG |
| | | CCTCGGTGAGTTTTCTCCTTCATTACAGAAACGGCTTTTTCAAAAA |
| | | TATGGTATTGATAATCCTGATATGAATAAATTGCAGTTTCATTTGAT |
| | | GCTCGATGAGTTTTTCTAACTGTCAGACCAAGTTTACTCATATATA |
| | | CTTTAGATTGATTTAAAACTTCATTTTTAATTTAAAAGGATCTAGGT |
| | | GAAGATCCTTTTTGATAATCTCATGACCAAAATCCCTTAACGTGAG |
| | | TTTTCGTTCCACTGAGCGTCAGACCCCGTAGAAAAGATCAAAGGA |
| | | TCTTCTTGAGATCCTTTTTTTCTGCGCGTAATCTGCTGCTTGCAAA |
| | | CAAAAAAACCACCGCTACCAGCGGTGGTTTGTTTGCCGGATCAAG |
| | | AGCTACCAACTCTTTTTCCGAAGGTAACTGGCTTCAGCAGAGCGC |
| | | AGATACCAAATACTGTTCTTCTAGTGTAGCCGTAGTTAGGCCACC |
| | | ACTTCAAGAACTCTGTAGCACCGCCTACATACCTCGCTCTGCTAA |
| | | TCCTGTTACCAGTGGCTGCTGCCAGTGGCGATAAGTCGTGTCTTA |
| | | CCGGGTTGGACTCAAGACGATAGTTACCGGATAAGGCGCAGCGG |
| | | TCGGGCTGAACGGGGGGTTCGTGCACACAGCCCAGCTTGGAGC |
| | | GAACGACCTACACCGAACTGAGATACCTACAGCGTGAGCTATGAG |
| | | AAAGCGCCACGCTTCCCGAAGGGAGAAAGGCGGACAGGTATCCG |
| | | GTAAGCGGCAGGGTCGGAACAGGAGAGCGCACGAGGGAGCTTC |
| | | CAGGGGGAAACGCCTGGTATCTTTATAGTCCTGTCGGGTTTCGCC |
| | | ACCTCTGACTTGAGCGTCGATTTTTGTGATGCTCGTCAGGGGGGC |
| | | GGAGCCTATGGAAAAACGCCAGCAACGCGGCCTTTTTACGGTTC |
| | | CTGGCCTTTTGCTGGCCTTTTGCTCACATGTTCTTTCCTGCGTTAT |
| | | CCCCTGATTCTGTGGATAACCGTATTACCGCCTTTGAGTGAGCTG |
| | | ATACCGCTCGCCGCAGCCGAACGACCGAGCGCAGCGAGTCAGT |
| | | GAGCGAGGAAGCGGAAGAGCGCCCAATACGCAAACCGCCTCTCC |
| | | CCGCGCGTTGGCCGATTCATTAATGCAGCTGGCACGACAGGTTT |
| | | CCCGACTGGAAAGCGGGCAGTGAGCGCAACGCAATTAATGTGAG |
| | | TTAGCTCACTCATTAGGCACCCCAGGCTTTACACTTTATGCTTCCG |
| | | GCTCGTATGTTGTGTGGAATTGTGAGCGGATAACAATTTCACACA |
| | | GGAAACAGCTATGACCATGATTACGCCAGATTTAATTAAGGCCTTA |
| | | ATTAGG |
| 46 | Plasmid P726 | CCTTAATTAGGCTGCGCGCTCGCTCGCTCACTGAGGCCGCCCGG |
| | 5′ ITR at nucleotide | GCAAAGCCCGGGCGTCGGGCGACCTTTGGTCGCCCGGCCTCAGT |
| | positions 12-141 | GAGCGAGCGAGCGCGCAGAGAGGGAGTGGCCAACTCCATCACTA |
| | AP head sequence at | GGGGTTCCTTGTAGTTAATGATTAACCCGCCATGCTACTTATCTAC |
| | nucleotide positions | GTAGCCATGCTCTAGGAAGATCGGAATTCGCCCTTAAGCTAGCGG |
| | 257-543 | CGCGCCCAATTGGCTTCGAATTCTAGCGGCCGCCCCCGGGTGCG |
| | AP splice acceptor at | CGGCGTCGGTGGTGCCGGGGGGGGCGCCAGGTCGCAGGCGGT |
| | nucleotide positions | GTAGGGCTCCAGGCAGGCGGCGAAGGCCATGACGTGCGCTATGA |
| | 566-614 | AGGTCTGCTCCTGCACGCCGTGAACCAGGTGCGCCTGCGGGCCG |
| | C-terminal STRC coding | CGCGCGAACACCGCCACGTCCTCGCCTGCGTGGGTCTCTTCGTC |
| | sequence at nucleotide | CAGGGGCACTGCTGACTGCTGCCGATACTCGGGGCTCCCGCTCT |
| | positions 615-3344 | CGCTCTCGGTAACATCCGGCCGGGCGCCGTCCTTGAGCACATAG |
| | bGH polyA sequence at | CCTGGACCGTTTCCTTAAGCGACGCATGCTCGCGATAGGCACCTA |
| | nucleotide positions | TTGGTCTTACTGACATCCACTTTGCCTTTCTCTCCACAGGAGCTGC |
| | 3376-3597 | TGTGGGCAGCCCTGCCTCTGCTTCCCCATCTGCCTCTGGAGAGCT |
| | 3′ ITR at nucleotide | TTCTCCAGCTCAGCCCTCACCAGATCCAGGCCCTGGAGGATAGCT |
| | positions 3685-3814 | GGCCAGTAGCAGATCTTGGGCCGGGACACGCCCGACATGTGCTT |
| | | CGTAGCCTAGTAAACCAGAGCATGGAGGATGGGGAGGAGCAGGT |
| | | GCTCAGGCTTGGGTCCCTCGCCTGTTTCCTGAGTCCTGAGGAGCT |
| | | ACAGAGTCTGGTGCCCTTGAGTGATCCAATGGGGCCTGTAGAACA |
| | | GGGTCTGCTGGAATGTGCGGCCAATGGGACCCTCAGCCCAGAAG |
| | | GACGGGTGGCATATGAACTTCTGGGAGTGTTGCGTTCATCTGGAG |
| | | GAACTGTCTTAAGCCCCCGAGAGCTGAGGGTCTGGGCACCTCTCT |
| | | TTCCCCAGCTGGGCCTCCGCTTCCTGCAGGAGCTCTCAGAGACCC |
| | | AGCTTAGAGCCATGCTTCCTGCCCTACAGGGAGCCAGTGTCACAC |
| | | CTGCCCAGGCTGTTCTGTTGTTTGGAAGGCTCCTTCCTAAGCATGA |
| | | TCTGTCCCTGGAGGAACTCTGCTCCCTGCACCCTCTCCTGCCAGG |
| | | TCTCAGCCCCCAGACACTCCAGGCCATCCCTAAGAGAGTTCTGGT |
| | | TGGTGCTTGTTCCTGCCTGGGCCCTGAACTGTCAAGGCTTTCAGC |
| | | TTGCCAGATTGCAGCTCTGCTGCAGACCTTTCGGGTAAAAGATGG |
| | | TGTTAAAAATATGGGTGCAGCAGGTGCCGGCTCAGCCGTGTGCAT |
| | | TCCTGGGCAGCCCACCACTTGGCCAGACTGCCTGCTTCCCCTGCT |
| | | CCCATTAAAGCTGCTACAGCTGGACGCTGCAGCTCTTCTGGCAAA |
| | | CCGAAGACTCTATCGGCAGCTGCCTTGGTCTGAGCAACAGGCACA |
| | | GTTTCTCTGGAAGAAAATGCAAGTGCCTACCAACCTGAGCCTGAG |
| | | GAATCTGCAGGCTCTGGGCAACTTGGCAGGAGGCATGACCTGCG |
| | | AGTTTCTGCAGCAGATCAGCTCAATGGTTGACTTTCTTGATGTGGT |
| | | ACACATGCTCTACCAGCTGCCCACTGGTGTTCGAGAGAGCCTGCG |
| | | GGCCTGTATCTGGACAGAGCTACAGCGGAGGATGACAATGCCAGA |
| | | GCCAGAGCTGACCACCCTAGGGCCAGAACTGAGTGAACTTGACAC |
| | | AAAGCTACTCCTGGACTTGCCGATCCAGCTGATGGACAGATTGTC |
| | | CAATGATTCCATTATGTTGGTGGTGGAGATGGTCCAAGGCGCTCC |
| | | AGAGCAGCTGCTGGCACTGACCCCACTCCACCAGACAGCCTTGG |
| | | CAGAGCGAGCACTTAAAAACCTGGCTCCAAAGGAGACCCCAATCT |
| | | CCAAAGAAGTGCTGGAGACACTGGGCCCCTTGGTTGGATTCCTGG |
| | | GAATAGAGAGCACGCGACGGATCCCTTTACCCATTCTACTGTCTCA |
| | | TCTCAGTCAGCTGCAGGGCTTCTGCCTAGGAGAGACATTTGCCAC |
| | | AGAGCTGGGATGGCTGCTGTTGCAGGAGCCTGTTCTTGGAAAACC |
| | | AGAATTGTGGAGCCAGGATGAAATAGAGCAAGCTGGACGCCTAGT |
| | | ATTCACTCTGTCTGCTGAGGCTATTTCCTCGATCCCCAGGGAGGC |
| | | TTTGGGCCCAGAGACACTGGAGAGGCTTCTGGGAAAGCATCAAAG |
| | | CTGGGAGCAGAGCAGAGTGGGCCATCTGTGTGGGGAGTCACAGC |
| | | TTGCCCACAAGAAAGCAGCTCTGGTAGCTGGGATTGTGCATCCAG |
| | | CTGCTGAGGGTCTCCAAGAGCCTGTACCAAACTGTGCAGACATAC |
| | | GGGGAACCTTCCCAGCGGCCTGGTCTGCGACACAAATCTCAGAGA |
| | | TGGAACTCTCAGACTTTGAAGACTGCCTGTCACTATTTGCTGGAGA |
| | | TCCAGGACTTGGTCCTGAGGAACTACGGGCAGCCATGGGCAAGG |
| | | CCAAGCAGTTGTGGGGTCCCCCTCGAGGATTCCGTCCTGAGCAGA |
| | | TCTTGCAGCTGGGCCGTCTCCTGATAGGTCTAGGAGAACGGGAAC |
| | | TGCAGGAGCTTACCTTGGTGGACTGGGGTGTGCTGAGCAGCCTG |
| | | GGGCAAATAGATGGCTGGAGTTCCATGCAGCTCCGAGCCGTGGT |
| | | CTCCAGTTTCCTAAGGCAGAGTGGTCGGCATGTGAGCCACCTGGA |
| | | CTTCATTTATCTGACAGCACTGGGTTACACAGTCTGTGGATTGCGA |
| | | CCAGAGGAGTTACAGCACATCAGCAGTTGGGAGTTTAGCCAAGCA |
| | | GCTCTCTTCCTGGGTAGCTTGCATCTCCCGTGCTCTGAGGAACAG |
| | | CTGGAAGTTCTGGCCTATCTCCTTGTGTTGCCTGGTGGCTTTGGC |
| | | CCAGTCAGTAACTGGGGGCCTGAGATCTTCACTGAAATTGGCACA |
| | | ATAGCAGCTGGCATCCCAGACCTGGCTCTTTCAGCATTACTGCGG |
| | | GGACAGATCCAAGGCCTGACTCCTCTTGCCATTTCTGTCATTCCTG |
| | | CTCCCAAGTTTGCAGTGGTCTTCAACCCCATCCAGTTATCTAGTCT |
| | | CACCAGGGGTCAGGCCGTAGCTGTTACTCCTGAACAGCTGGCCTA |
| | | TCTGAGTCCTGAGCAGCGGCGAGCAGTTGCATGGGCCCAACACG |
| | | AAGGGAAGGAGATCCCAGAGCAGCTGGGTCGAAACTCAGCCTGG |
| | | GGTCTCTACGACTGGTTCCAAGCCTCCTGGGCCCTGGCATTGCCC |
| | | GTCAGCATTTTTGGCCACCTATTATGAGCGGCCGCGGTACCAAGG |
| | | GCGGATCCTGCATAGAGCTCGCTGATCAGCCTCGACTGTGCCTTC |
| | | TAGTTGCCAGCCATCTGTTGTTTGCCCCTCCCCCGTGCCTTCCTTG |
| | | ACCCTGGAAGGTGCCACTCCCACTGTCCTTTCCTAATAAAATGAGG |
| | | AAATTGCATCGCATTGTCTGAGTAGGTGTCATTCTATTCTGGGGGG |
| | | TGGGGTGGGGCAGGACAGCAAGGGGGAGGATTGGGAAGACAATA |
| | | GCAGGCATCTCGAGTTAAGGGCGAATTCCCGATAAGGATCTTCCT |
| | | AGAGCATGGCTACGTAGATAAGTAGCATGGGGGGTTAATCATTAA |
| | | CTACAAGGAACCCCTAGTGATGGAGTTGGCCACTCCCTCTCTGCG |
| | | CGCTCGCTCGCTCACTGAGGCCGGGCGACCAAAGGTCGCCCGAC |
| | | GCCCGGGCTTTGCCCGGGGGCCTCAGTGAGCGAGCGAGCGCG |
| | | CAGCCTTAATTAACCTAATTCACTGGCCGTCGTTTTACAACGTCGT |
| | | GACTGGGAAAACCCTGGCGTTACCCAACTTAATCGCCTTGCAGCA |
| | | CATCCCCCTTTCGCCAGCTGGCGTAATAGCGAAGAGGCCCGCACC |
| | | GATCGCCCTTCCCAACAGTTGCGCAGCCTGAATGGCGAATGGGAC |
| | | GCGCCCTGTAGCGGCGCATTAAGCGCGGCGGGTGTGGTGGTTAC |
| | | GCGCAGCGTGACCGCTACACTTGCCAGCGCCCTAGCGCCCGCTC |
| | | CTTTCGCTTTCTTCCCTTCCTTTCTCGCCACGTTCGCCGGCTTTCC |
| | | CCGTCAAGCTCTAAATCGGGGGCTCCCTTTAGGGTTCCGATTTAG |
| | | TGCTTTACGGCACCTCGACCCCAAAAAACTTGATTAGGGTGATGGT |
| | | TCACGTAGTGGGCCATCGCCCTGATAGACGGTTTTTCGCCCTTTG |
| | | ACGTTGGAGTCCACGTTCTTTAATAGTGGACTCTTGTTCCAAACTG |
| | | GAACAACACTCAACCCTATCTCGGTCTATTCTTTTGATTTATAAGGG |
| | | ATTTTGCCGATTTCGGCCTATTGGTTAAAAAATGAGCTGATTTAACA |
| | | AAAATTTAACGCGAATTTTAACAAAATATTAACGCTTACAATTTAGG |
| | | TGGCACTTTTCGGGGAAATGTGCGCGGAACCCCTATTTGTTTATTT |
| | | TTCTAAATACATTCAAATATGTATCCGCTCATGAGACAATAACCCTG |
| | | ATAAATGCTTCAATAATATTGAAAAAGGAAGAGTATGAGCCATATTC |
| | | AACGGGAAACGTCGAGGCCGCGATTAAATTCCAACATGGATGCTG |
| | | ATTTATATGGGTATAAATGGGCTCGCGATAATGTCGGGCAATCAGG |
| | | TGCGACAATCTATCGCTTGTATGGGAAGCCCGATGCGCCAGAGTT |
| | | GTTTCTGAAACATGGCAAAGGTAGCGTTGCCAATGATGTTACAGAT |
| | | GAGATGGTCAGACTAAACTGGCTGACGGAATTTATGCCTCTTCCG |
| | | ACCATCAAGCATTTTATCCGTACTCCTGATGATGCATGGTTACTCA |
| | | CCACTGCGATCCCCGGAAAAACAGCATTCCAGGTATTAGAAGAAT |
| | | ATCCTGATTCAGGTGAAAATATTGTTGATGCGCTGGCAGTGTTCCT |
| | | GCGCCGGTTGCATTCGATTCCTGTTTGTAATTGTCCTTTTAACAGC |
| | | GATCGCGTATTTCGTCTTGCTCAGGCGCAATCACGAATGAATAACG |
| | | GTTTGGTTGATGCGAGTGATTTTGATGACGAGCGTAATGGCTGGC |
| | | CTGTTGAACAAGTCTGGAAAGAAATGCATAAACTTTTGCCATTCTC |
| | | ACCGGATTCAGTCGTCACTCATGGTGATTTCTCACTTGATAACCTT |
| | | ATTTTTGACGAGGGGAAATTAATAGGTTGTATTGATGTTGGACGAG |
| | | TCGGAATCGCAGACCGATACCAGGATCTTGCCATCCTATGGAACT |
| | | GCCTCGGTGAGTTTTCTCCTTCATTACAGAAACGGCTTTTTCAAAA |
| | | ATATGGTATTGATAATCCTGATATGAATAAATTGCAGTTTCATTTGA |
| | | TGCTCGATGAGTTTTTCTAACTGTCAGACCAAGTTTACTCATATATA |
| | | CTTTAGATTGATTTAAAACTTCATTTTTAATTTAAAAGGATCTAGGTG |
| | | AAGATCCTTTTTGATAATCTCATGACCAAAATCCCTTAACGTGAGTT |
| | | TTCGTTCCACTGAGCGTCAGACCCCGTAGAAAAGATCAAAGGATC |
| | | TTCTTGAGATCCTTTTTTTCTGCGCGTAATCTGCTGCTTGCAAACAA |
| | | AAAAACCACCGCTACCAGCGGTGGTTTGTTTGCCGGATCAAGAGC |
| | | TACCAACTCTTTTTCCGAAGGTAACTGGCTTCAGCAGAGCGCAGAT |
| | | ACCAAATACTGTTCTTCTAGTGTAGCCGTAGTTAGGCCACCACTTC |
| | | AAGAACTCTGTAGCACCGCCTACATACCTCGCTCTGCTAATCCTGT |
| | | TACCAGTGGCTGCTGCCAGTGGCGATAAGTCGTGTCTTACCGGGT |
| | | TGGACTCAAGACGATAGTTACCGGATAAGGCGCAGCGGTCGGGC |
| | | TGAACGGGGGGTTCGTGCACACAGCCCAGCTTGGAGCGAACGAC |
| | | CTACACCGAACTGAGATACCTACAGCGTGAGCTATGAGAAAGCGC |
| | | CACGCTTCCCGAAGGGAGAAAGGCGGACAGGTATCCGGTAAGCG |
| | | GCAGGGTCGGAACAGGAGAGCGCACGAGGGAGCTTCCAGGGGG |
| | | AAACGCCTGGTATCTTTATAGTCCTGTCGGGTTTCGCCACCTCTGA |
| | | CTTGAGCGTCGATTTTTGTGATGCTCGTCAGGGGGGCGGAGCCTA |
| | | TGGAAAAACGCCAGCAACGCGGCCTTTTTACGGTTCCTGGCCTTT |
| | | TGCTGGCCTTTTGCTCACATGTTCTTTCCTGCGTTATCCCCTGATT |
| | | CTGTGGATAACCGTATTACCGCCTTTGAGTGAGCTGATACCGCTC |
| | | GCCGCAGCCGAACGACCGAGCGCAGCGAGTCAGTGAGCGAGGAA |
| | | GCGGAAGAGCGCCCAATACGCAAACCGCCTCTCCCCGCGCGTTG |
| | | GCCGATTCATTAATGCAGCTGGCACGACAGGTTTCCCGACTGGAA |
| | | AGCGGGCAGTGAGCGCAACGCAATTAATGTGAGTTAGCTCACTCA |
| | | TTAGGCACCCCAGGCTTTACACTTTATGCTTCCGGCTCGTATGTTG |
| | | TGTGGAATTGTGAGCGGATAACAATTTCACACAGGAAACAGCTATG |
| | | ACCATGATTACGCCAGATTTAATTAAGG |

Other exemplary pairs of overlapping, trans-splicing, and dual hybrid vectors are described in Table 6 below.

TABLE 6
Representative pairs of overlapping, trans-splicing, and hybrid dual vectors for use in
the methods and compositions described herein

| Vector | | |
|---|---|---|
| Pair | | |
| Number | Vector Type | Vector Pair |
| 1 | Overlapping | First nucleic acid vector contains: an OCM promoter (e.g., SEQ ID NO: 1) |
| | | operably linked to a polynucleotide encoding an N-terminal portion of a |
| | | human stereocilin protein (an N-terminal portion of SEQ ID NO: 4), in |
| | | which the polynucleotide encoding the N-terminal portion of the human |
| | | stereocilin protein includes the 500 bp 3′ of the position selected as the |
| | | central point of the overlapping region of STRC |
| | | Second nucleic acid vector contains: a polynucleotide encoding a C- |
| | | terminal portion of the human stereocilin protein and a poly(A) sequence, |
| | | in which the polynucleotide encoding the C-terminal portion of the human |
| | | stereocilin protein includes the 500 bp 5′ of the position selected as the |
| | | central point of the overlapping region of STRC |
| 2 | Trans- | First nucleic acid vector contains: an OCM promoter (e.g., SEQ ID NO: 1) |
| | splicing | operably linked to a polynucleotide encoding an N-terminal portion of a |
| | | human stereocilin protein (an N-terminal portion of SEQ ID NO: 4) and a |
| | | splice donor sequence 3′ of the polynucleotide |
| | | Second nucleic acid vector contains: a splice acceptor sequence 5′ of a |
| | | polynucleotide encoding a C-terminal portion of the human stereocilin |
| | | protein and a poly(A) sequence |
| 3 | Hybrid | First nucleic acid vector contains: an OCM promoter (e.g., SEQ ID NO: 1) |
| | | operably linked to a polynucleotide encoding an N-terminal portion of a |
| | | human stereocilin protein (e.g., an N-terminal portion of SEQ ID NO: 4), a |
| | | splice donor sequence 3′ of the polynucleotide, and a recombinogenic |
| | | region 3′ of the splice donor sequence |
| | | Second nucleic acid vector contains: a recombinogenic region, a splice |
| | | acceptor sequence 3′ of the recombinogenic region, a polynucleotide |
| | | encoding a C-terminal portion of the human stereocilin protein 3′ of the |
| | | splice acceptor sequence, and a poly(A) sequence |

### Intein Expression Systems

Another gene therapy approach for expressing large proteins in mammalian cells involves the use of inteins. An intein, also known as a “protein intron,” is a portion of a protein that is typically 100-900 amino acid residues long and is capable of self-excision and ligation of the N- and C-terminal residues of the flanking protein fragments (“exteins”). Inteins can be divided into three different classes, including maxi-intein, mini-intein, and split intein. Maxi-inteins refer to N- and C-terminal splicing regions of a protein interrupted by a homing endonuclease domain (HEG). HEGs refer to a class of endonucleases encoded as stand-alone genes within introns, as protein fusions with other proteins, or as self-splicing inteins. HEGs generally hydrolyze very few and select DNA regions. Once a HEG hydrolyzes a piece of DNA, the gene encoding the HEG typically incorporates itself into the cleavage site, thereby increasing its allele frequency. “Mini-inteins” refer to N- and C-terminal splicing domains lacking the HEG domain. “Split inteins” refer to inteins that are transcribed and translated as two separate polypeptides that are joined with an extein. Alanine inteins are another class of inteins that have a splicing junction of an alanine instead of a cysteine or serine.

The splicing domain of inteins contains two subdomains, namely the N- and C-terminal splicing domains, which contain conserved motifs with conserved residues that mediate the splicing activity. The N-terminal splicing domain contains A, N2, B, and N4 structural motifs, whereas the C-terminal splicing domain contains F and G motifs. The A-motif contains Cys/Ser or Thr as conserved residues; the B motif includes His and Thr residues; F motif contains Asp and His residues; G motifs carry two conserved residues, which include a penultimate His and a terminal Asn. C, D, E, and H motifs are generally related to the HEG domain in maxi-inteins.

Intein splicing falls within three distinct strategies: 1) class 1 (or classical/canonical) intein splicing which involves (a) a (N-S/N-O) acyl shift that transforms the peptide bond of an N-terminal splice junction to a thio(ester) linkage, (b) transesterification reaction that forms a branched intermediate, (c) Asn cyclization, which removes the branched intermediate by cleaving the C-terminal splice junction, and (d) a second (S-N/O-N) acyl shift that ligates the flanking extein segments through amide bond formation; 2) class 2 inteins (also known as Alanine-inteins) bypass step (a) of the classical splicing reaction; and 3) class 3 mechanism which involves the formation of two branched intermediates.

Among the various intein systems described above, the split intein trans-splicing approach has been demonstrated to successfully overcome the size limitations of traditional gene therapy vectors (e.g., AAV: −5 kb maximal size limit). For example, Subramanyam et al. (PNAS 110:15461-6 (2013)) have employed the split intein system to reconstitute the a1 C-subunit of L-type calcium channel in cardiomyocytes from two separate halves. Similarly, Truong et al. (Nucleic Acids Res. 43:6450-8 (2015)) have shown successful reconstitution of two halves of the Cas9 protein using a split intein system. Accordingly, the present disclosure provides split intein trans-splicing systems for the packaging and delivery of a stereocilin coding sequence that is operably linked to an OCM promoter. This method allows for two separate polynucleotides, each containing approximately one half of the STRC gene and including a polynucleotide sequence encoding an N-intein fragment or a C-intein fragment, to be expressed from two separate expression vectors (e.g., any one of the nucleic acid vectors disclosed herein) and post-translationally reconstituted to produce a full-length stereocilin protein. Such systems may be incorporated into nucleic acid expression vectors disclosed herein, such as, e.g., rAAV vectors.

In one example, the present disclosure provides a two-vector split intein system containing: a) a first nucleic acid vector containing a polynucleotide that includes a sequence encoding an N-terminal portion of a human stereocilin protein (e.g., an N-terminal portion of SEQ ID NO: 4), in which the sequence encoding an N-terminal portion of a stereocilin protein includes at its 3′ end a polynucleotide sequence encoding an N-intein; b) a second vector containing a polynucleotide that includes a sequence encoding a C-terminal portion of a human stereocilin protein (e.g., a C-terminal portion of SEQ ID NO: 4), in which the sequence encoding a C-terminal portion of an stereocilin protein includes at its 5′ end a polynucleotide sequence encoding a C-intein.

In another example, the present disclosure provides a two-vector split intein system containing: a) a first vector containing a polynucleotide that includes a sequence encoding an N-terminal portion of a murine stereocilin protein (e.g., an N-terminal portion of SEQ ID NO: 5), in which the sequence encoding an N-terminal portion of a stereocilin protein includes at its 3′ end a polynucleotide sequence encoding an N-intein; b) a second vector containing a polynucleotide that includes a sequence encoding a C-terminal portion of a murine stereocilin protein (e.g., a C-terminal portion of SEQ ID NO: 5), in which the sequence encoding a C-terminal portion of a stereocilin protein includes at its 5′ end a nucleic acid sequence encoding a C-intein.

In some embodiments, both the first vector and the second vector further include a promoter sequence, such as an OCM promoter sequence (e.g., an OCM promoter sequence of any one of SEQ ID NOs: 1-3) operably linked to the 5′ end of a polynucleotide encoding the first fusion protein (an N-terminal portion of a stereocilin protein fused to an N-intein) and/or to the 5′ end of the polynucleotide encoding the second fusion protein (a C-terminal portion of a stereocilin protein fused to a C-intein). In some embodiments, the OCM promoter has the sequence of SEQ ID NO: 1 or a variant thereof having at least 85% (e.g., at least 86%, 87%, 88%, 89%, 90%, 91%, 92%, 93%, 94%, 95%, 96%, 97%, 98%, 99%, or more) sequence identity to SEQ ID NO: 1. In some embodiments, the OCM promoter has the sequence of SEQ ID NO: 2 or a variant thereof having at least 85% (e.g., at least 86%, 87%, 88%, 89%, 90%, 91%, 92%, 93%, 94%, 95%, 96%, 97%, 98%, 99%, or more) sequence identity to SEQ ID NO: 2. In some embodiments, the OCM promoter has the sequence of SEQ ID NO: 3 or a variant thereof having at least 85% (e.g., at least 86%, 87%, 88%, 89%, 90%, 91%, 92%, 93%, 94%, 95%, 96%, 97%, 98%, 99%, or more) sequence identity to SEQ ID NO: 3.

In some embodiments, the N-intein and the C-intein are derived from the same intein or split intein gene. Alternatively, the N-intein and the C-intein sequences derive from two different intein genes that can perform protein trans-splicing to reconstitute a full-length stereocilin protein. In some embodiments, the same gene is from the same organism or from different organisms. Commonly used split inteins derive from the DnaEgene from various organisms. In some embodiments, the polynucleotide encoding a stereocilin protein is split into two portions, each corresponding to approximately half of the total coding sequence of the full-length gene, namely a N-terminal portion and a C-terminal portion. The polynucleotide encoding the N-terminal portion of stereocilin is fused in frame at its 3′ end with the polynucleotide encoding the N-intein, whereas the polynucleotide encoding the C-terminal portion of stereocilin is fused in frame at its 5′ end with the polynucleotide encoding the C-intein.

In some embodiments, the first vector and the second vector, when introduced into a cell (e.g., a cell of a subject, such as a subject with sensorineural hearing loss, e.g., DFNB16) produce a first fusion protein and a second fusion protein. In some embodiments, the first fusion protein contains the N-terminal portion of the stereocilin protein fused at its C-terminus with the N-intein. In some embodiments, the second fusion protein contains the C-terminal portion of the stereocilin protein fused at its N-terminus with the C-intein. In some embodiments, the N-intein of the first fusion protein and the C-intein of the second fusion protein selectively bind to produce a third fusion protein containing from N-terminus to C-terminus: an N-terminal portion of the stereocilin protein, an N-intein bound at its C-terminus to the C-intein, and the C-terminal portion of the stereocilin protein. In some embodiments, the N-intein bound to the C-intein is capable of performing a trans-splicing reaction that excises the N-intein and the C-intein and ligates of the C-terminus of the N-terminal portion and the N-terminus of the C-terminal portion of the stereocilin protein.

The split intein system described herein may include split inteins that are encoded by one gene that is subsequently engineered using routine methods to encode two separate intein fragments (e.g., a split intein). In some embodiments, the split inteins are encoded by two separate genes.

Split inteins of the disclosed compositions and methods may be derived from the DnaEgene (e.g., DNA polymerase III subunit alpha) from cyanobacteria, such as, e.g., Nostoc punctiforme (Npu), Synechocystis sp. PCC6803 (Ssp), Fischerella sp. PCC9605 (Fsp), Scytonema tolypothrichoides (Sto), Cyanobacteria bacterium SW_9_47_5, Nodularia spumigena (Nsp), Nostoc flagelliforme (Nfl), Crocosphaera watsonii (Cwa) WH8502, Chroococcidiopsis cubana (Ccu) CCALA043, Trichodesmium erythraeum (Ter), Rhodothermus marinus (Rma), Saccharomyces cerevisiae (Sce), Saccharomyces castellii (Sca), Saccharomyces unisporus (Sun), Zygosaccharomyces bisporus (Zbi), Torulaspora pretoriensis (Tpr), Mycobacteria tuberculosis (Mtu), Mycobacterium leprae (Mle), Mycobacterium smegmatis (Msm), Pyrococcus abyssi (Pab), Pyrococcus horikoshii (Pho), Coxiella burnetti (Cbu), Coxiella neoformans (Cne), Coxiella gattii (Cga), Histoplasma capsulatum (Hca), and Porphyra purpurea chloroplast (Ppu), among others. In some embodiments, the split intein is derived from multiple sequence alignment studies of DnaE for identifying a consensus design (e.g., Cfa) to engineer a split intein with desirable stability and activity (e.g., the split inteins are Cfa inteins). Other split intein systems suitable for use with the presently disclosed compositions and methods include those described in International Patent Application Publication Nos. WO 2017/132580, WO 2020/079034, WO 2018/071868, WO 2020/249723, WO 2021/099607, WO 2021/040703, WO 2013/045632, WO 2020/146627, and WO 2021/047558, and U.S. Pat. Nos. 10,066,027, 10,526,401, and 8,394,604, each of which is incorporated herein by reference herein as it relates to split intein systems.

In some embodiments, the first vector and the second vector further include a 5′ inverted terminal repeat (ITR) at its 5′ end and a 3′ ITR and its 3′ end. In some embodiments, the 5′ ITR and the 3′ ITR are AAV ITRs. In some embodiments, the AAV ITRs are AAV2 ITRs.

In some embodiments, the two-vector split intein system of the disclosure includes: a) a first vector containing from 5′ to 3′: i) optionally, a 5′ ITR (e.g., AAV2 5′ ITR); ii) a polynucleotide containing an OCM promoter (e.g., an OCM promoter of any one of SEQ ID NOs: 1-3); iii) a polynucleotide encoding an N-terminal portion of a stereocilin protein (e.g., an N-terminal portion of the stereocilin protein of SEQ ID NO: 4 or SEQ ID NO: 5); iv) a polynucleotide encoding an N-intein; (v) optionally, a poly(A) sequence; and (vi) optionally, a 3′ ITR (e.g., AAV2 3′ ITR); and b) a second vector containing from 5′ to 3′: i) optionally, a 5′ ITR (e.g., AAV2 5′ ITR); ii) a polynucleotide containing an OCM promoter (e.g., an OCM promoter of any one of SEQ ID NOs: 1-3); iii) a polynucleotide encoding a C-intein; iv) a polynucleotide encoding a C-terminal portion of the stereocilin protein (e.g., a C-terminal portion of the STRC protein of SEQ ID NO: 4 or SEQ ID NO: 5); (v) optionally, a poly(A) sequence; and (vi) optionally, a 3′ ITR (e.g., AAV2 3′ ITR).

In some embodiments, the two-vector split intein system of the disclosure includes a polynucleotide encoding an N-intein peptide having an amino acid sequence of SEQ ID NO: 8 or having at least 85% (e.g., at least 86%, 87%, 88%, 89%, 90%, 91%, 92%, 93%, 94%, 95%, 96%, 97%, 98%, 99%, or more) sequence identity to SEQ ID NO: 8, as is shown below.

(SEQ ID NO: 8)
CLSYDTEILTVEYGFLPIGKIVEERIECTVYTVDKNGFVYTQPIAQWHN
RGEQEVFEYCLEDGSIIRATKDHKFMTTDGQMLPIDEIFERGL

In some embodiments, the two-vector split intein system of the disclosure includes a polynucleotide encoding a C-intein peptide having an amino acid sequence of SEQ ID NO: 9 or having at least 85% (e.g., at least 86%, 87%, 88%, 89%, 90%, 91%, 92%, 93%, 94%, 95%, 96%, 97%, 98%, 99%, or more) sequence identity to SEQ ID NO: 9, as is shown below.

(SEQ ID NO: 9)
VKIISRKSLGTQNVYDIGVEKDHNFLLKNGLVASN

In some embodiments, the two-vector split intein system of the disclosure includes a polynucleotide encoding an N-intein peptide having an amino acid sequence of SEQ ID NO: 10 or having at least 85% (e.g., at least 86%, 87%, 88%, 89%, 90%, 91%, 92%, 93%, 94%, 95%, 96%, 97%, 98%, 99%, or more) sequence identity to SEQ ID NO: 10, as is shown below.

(SEQ ID NO: 10)
CLSYDTEILTVEYGFLPIGKIVEERIECTVYTVDKNGFVYTQPIAQWHN
RGEQEVFEYCLEDGSIIRATKDHKFMTTDGQMLPIDEIFERGLDLKQVD
GLP

In some embodiments, the two-vector split intein system of the disclosure includes a polynucleotide encoding a C-intein peptide having an amino acid sequence of SEQ ID NO: 11 or having at least 85% (e.g., at least 86%, 87%, 88%, 89%, 90%, 91%, 92%, 93%, 94%, 95%, 96%, 97%, 98%, 99%, or more) sequence identity to SEQ ID NO: 11, as is shown below.

(SEQ ID NO: 11)
MVKIISRKSLGTQNVYDIGVEKDHNFLLKNGLVASN

In some embodiments, the two-vector split intein system of the disclosure includes a polynucleotide encoding a C-intein peptide having an amino acid sequence of SEQ ID NO: 12 or having at least 85% (e.g., at least 86%, 87%, 88%, 89%, 90%, 91%, 92%, 93%, 94%, 95%, 96%, 97%, 98%, 99%, or more) sequence identity to SEQ ID NO: 12, as is shown below.

(SEQ ID NO: 12)
VKIISRKSLGTQNVYDIGVGEPHNFLLKNGLVASN

In some embodiments, the two-vector split intein system includes a first vector including a polynucleotide encoding an N-intein peptide having an amino acid sequence of SEQ ID NO: 8 or SEQ ID NO: 10 (e.g., positioned 3′ of a polynucleotide encoding an N-terminal portion of a stereocilin protein) and a second vector including a polynucleotide encoding a C-intein polypeptide having an amino acid sequence of SEQ ID NO: 9, SEQ ID NO: 11, or SEQ ID NO: 12 (e.g., positioned 5′ of a polynucleotide encoding a C-terminal portion of a stereocilin protein). In some embodiments, the two-vector split intein system includes a first vector including a polynucleotide encoding an N-intein peptide having an amino acid sequence of SEQ ID NO: 8 and a second vector including a polynucleotide encoding a C-intein polypeptide having an amino acid sequence of SEQ ID NO: 9. In some embodiments, the two-vector split intein system includes a first vector including a polynucleotide encoding an N-intein peptide having an amino acid sequence of SEQ ID NO: 8 and a second vector including a polynucleotide encoding a C-intein polypeptide having an amino acid sequence of SEQ ID NO: 11. In some embodiments, the two-vector split intein system includes a first vector including a polynucleotide encoding an N-intein peptide having an amino acid sequence of SEQ ID NO: 8 and a second vector including a polynucleotide encoding a C-intein polypeptide having an amino acid sequence of SEQ ID NO: 12. In some embodiments, the two-vector split intein system includes a first vector including a polynucleotide encoding an N-intein peptide having an amino acid sequence of SEQ ID NO: 10 and a second vector including a polynucleotide encoding a C-intein polypeptide having an amino acid sequence of SEQ ID NO: 9. In some embodiments, the two-vector split intein system includes a first vector including a polynucleotide encoding an N-intein peptide having an amino acid sequence of SEQ ID NO: 10 and a second vector including a polynucleotide encoding a C-intein polypeptide having an amino acid sequence of SEQ ID NO: 11. In some embodiments, the two-vector split intein system includes a first vector including a polynucleotide encoding an N-intein peptide having an amino acid sequence of SEQ ID NO: 10 and a second vector including a polynucleotide encoding a C-intein polypeptide having an amino acid sequence of SEQ ID NO: 12.

In some embodiments, the two-vector split intein system of the disclosure includes a polynucleotide encoding an N-intein peptide having an amino acid sequence of SEQ ID NO: 13 or having at least 85% (e.g., at least 86%, 87%, 88%, 89%, 90%, 91%, 92%, 93%, 94%, 95%, 96%, 97%, 98%, 99%, or more) sequence identity to SEQ ID NO: 13, as is shown below.

(SEQ ID NO: 13)
CLSYETEILTVEYGLLPIGKIVEKRIECTVYSVDNNGNIYTQPVAQWHD
RGEQEVFEYCLEDGSLIRATKDHKFMTVDGQMLPIDEIFERELDLMRVD
NLPN

In some embodiments, the two-vector split intein system of the disclosure includes a polynucleotide encoding a C-intein peptide having an amino acid sequence of SEQ ID NO: 14 or having at least 85% (e.g., at least 86%, 87%, 88%, 89%, 90%, 91%, 92%, 93%, 94%, 95%, 96%, 97%, 98%, 99%, or more) sequence identity to SEQ ID NO: 14, as is shown below.

(SEQ ID NO: 14)
MIKIATRKYLGKQNVYDIGVERDHNFALKNGFIASN

In some embodiments, the two-vector split intein system includes a first vector including a polynucleotide encoding an N-intein peptide having an amino acid sequence of SEQ ID NO: 13 (e.g., positioned 3′ of a polynucleotide encoding an N-terminal portion of a stereocilin protein) and a second vector including a polynucleotide encoding a C-intein polypeptide having an amino acid sequence of SEQ ID NO: 14 (e.g., positioned 5′ of a polynucleotide encoding a C-terminal portion of a stereocilin protein).

In some embodiments, the two-vector split intein system of the disclosure includes a polynucleotide encoding an N-intein peptide having an amino acid sequence of SEQ ID NO: 15 or having at least 85% (e.g., at least 86%, 87%, 88%, 89%, 90%, 91%, 92%, 93%, 94%, 95%, 96%, 97%, 98%, 99%, or more) sequence identity to SEQ ID NO: 15, as is shown below.

(SEQ ID NO: 15)
CLSYDTEILTVEYGFLPIGKIVEERIECTVYTVDKNGFVYTQPIAQWHN
RGEQEVFEYCLEDGSIIRATKDHKFMTTDGQMLPIDEIFERGLDLKQVD
GLP

In some embodiments, the two-vector split intein system of the disclosure includes a polynucleotide encoding a C-intein peptide having an amino acid sequence of SEQ ID NO: 16 or having at least 85% (e.g., at least 86%, 87%, 88%, 89%, 90%, 91%, 92%, 93%, 94%, 95%, 96%, 97%, 98%, 99%, or more) sequence identity to SEQ ID NO: 16, as is shown below.

(SEQ ID NO: 16)
MKRTADGSEFESPKKKRKVKIISRKSLGTQNVYDIGVEKDHNFLLKNGL
VASN

In some embodiments, the two-vector split intein system includes a first vector including a polynucleotide encoding an N-intein peptide having an amino acid sequence of SEQ ID NO: 15 (e.g., positioned 3′ of a polynucleotide encoding an N-terminal portion of a stereocilin protein) and a second vector including a polynucleotide encoding a C-intein polypeptide having an amino acid sequence of SEQ ID NO: 16 (e.g., positioned 5′ of a polynucleotide encoding a C-terminal portion of a stereocilin protein).

In some embodiments, the two-vector split intein system of the disclosure includes a polynucleotide encoding an N-intein peptide having an amino acid sequence of CFSGDTLVALTD (SEQ ID NO: 17). In some embodiments, the two-vector split intein system of the disclosure includes a polynucleotide encoding an N-intein peptide having an amino acid sequence of CLAGDTLITLA (SEQ ID NO: 18). In some embodiments, the two-vector split intein system of the disclosure includes a polynucleotide encoding an N-intein peptide having an amino acid sequence of CLQNGTRLLR (SEQ ID NO: 19). In some embodiments, the two-vector split intein system of the disclosure includes a polynucleotide encoding an N-intein peptide having an amino acid sequence of CLTGDSQVLTR (SEQ ID NO: 20). In some embodiments, the two-vector split intein system of the disclosure includes a polynucleotide encoding an N-intein peptide having an amino acid sequence of CLTYETEIMTV (SEQ ID NO: 21). In some embodiments, the two-vector split intein system of the disclosure includes a polynucleotide encoding an N-intein peptide having an amino acid sequence of CLSGNTKVRFRY (SEQ ID NO: 22). In some embodiments, the two-vector split intein system of the disclosure includes a polynucleotide encoding an N-intein peptide having an amino acid sequence that has least 85% (e.g., at least 86%, 87%, 88%, 89%, 90%, 91%, 92%, 93%, 94%, 95%, 96%, 97%, 98%, 99%, or more) sequence identity to any one of SEQ ID NOs: 17-22.

In some embodiments, the two-vector split intein system of the disclosure includes a polynucleotide encoding a C-intein peptide having an amino acid sequence of GVFVHN (SEQ ID NO: 23). In some embodiments, the two-vector split intein system of the disclosure includes a polynucleotide encoding a C-intein peptide having an amino acid sequence of GLLVHN (SEQ ID NO: 24). In some embodiments, the two-vector split intein system of the disclosure includes a polynucleotide encoding a C-intein peptide having an amino acid sequence of GLIASN (SEQ ID NO: 25). In some embodiments, the two-vector split intein system of the disclosure includes a polynucleotide encoding a C-intein peptide having an amino acid sequence of GLVVHN (SEQ ID NO: 26). In some embodiments, the two-vector split intein system of the disclosure includes a polynucleotide encoding a C-intein peptide having an amino acid sequence that has least 85% (e.g., at least 86%, 87%, 88%, 89%, 90%, 91%, 92%, 93%, 94%, 95%, 96%, 97%, 98%, 99%, or more) sequence identity to any one of SEQ ID NOs: 23-26.

In some embodiments, the two-vector split intein system includes a first vector including a polynucleotide encoding an N-intein peptide having an amino acid sequence of SEQ ID NO: 17 (e.g., positioned 3′ of a polynucleotide encoding an N-terminal portion of a stereocilin protein) and a second vector including a polynucleotide encoding a C-intein polypeptide having an amino acid sequence of SEQ ID NO: 23 (e.g., positioned 5′ of a polynucleotide encoding a C-terminal portion of a stereocilin protein). In some embodiments, the two-vector split intein system includes a first vector including a polynucleotide encoding an N-intein peptide having an amino acid sequence of SEQ ID NO: 20 (e.g., positioned 3′ of a polynucleotide encoding an N-terminal portion of a stereocilin protein) and a second vector including a polynucleotide encoding a C-intein polypeptide having an amino acid sequence of SEQ ID NO: 24 (e.g., positioned 5′ of a polynucleotide encoding a C-terminal portion of a stereocilin protein). In some embodiments, the two-vector split intein system includes a first vector including a polynucleotide encoding an N-intein peptide having an amino acid sequence of SEQ ID NO: 21 (e.g., positioned 3′ of a polynucleotide encoding an N-terminal portion of a stereocilin protein) and a second vector including a polynucleotide encoding a C-intein polypeptide having an amino acid sequence of SEQ ID NO: 25 (e.g., positioned 5′ of a polynucleotide encoding a C-terminal portion of a stereocilin protein). In some embodiments, the two-vector split intein system includes a first vector including a polynucleotide encoding an N-intein peptide having an amino acid sequence of SEQ ID NO: 22 (e.g., positioned 3′ of a polynucleotide encoding an N-terminal portion of a stereocilin protein) and a second vector including a polynucleotide encoding a C-intein polypeptide having an amino acid sequence of SEQ ID NO: 26 (e.g., positioned 5′ of a polynucleotide encoding a C-terminal portion of a stereocilin protein).

In some embodiments, the two-vector split intein system of the disclosure collectively includes one or more polynucleotides encoding an N-intein and C-intein pair described in Table 7 or one or more polynucleotides encoding an N-intein and C-intein pair having at least 85% sequence identity (e.g., at least 86%, 87%, 88%, 89%, 90%, 91%, 92%, 93%, 94%, 95%, 96%, 97%, 98%, 99%, or more) sequence identity to an N-intein and C-intein pair described in Table 7, as is shown below. In some embodiments, the two-vector split intein system includes a first vector including a polynucleotide encoding an N-intein peptide having an amino acid sequence listed in Table 7 (e.g., positioned 3′ of a polynucleotide encoding an N-terminal portion of a stereocilin protein) and a second vector including a polynucleotide encoding a C-intein polypeptide having an amino acid sequence listed in the same row of Table 7 as the N-intein amino acid sequence (e.g., positioned 5′ of a polynucleotide encoding a C-terminal portion of a stereocilin protein).

TABLE 7
Representative split intein sequence pairs

| | N-intein | SEQ | C-intein | SEQ |
|---|---|---|---|---|
| Intein | amino acid sequence | ID NO | amino acid sequence | ID NO |
| Npu- | CLSYETEILTVEYGLLPIGKIVEKRIECT | 27 | IKIATRKYLGKQNVYDIGVERDH | 28 |
| DnaE | VYSVDNNGNIYTQPVAQWHDRGEQE | | NFALKNGFIASN | |
| | VFEYCLEDGSLIRATKDHKFMTVDGQ | | | |
| | MLPIDEIFERELDLMRVDNLPN | | | |
| Rma- | CLAGDTLITLADGRRVPIRELVSQQNF | 29 | AAACPELRQLAQSDVYWDPIV | 30 |
| DnaB | SVWALNPQTYRLERARVSRAFCTGIK | | SIEPDGVEEVFDLTVPGPHNFV | |
| | PVYRLTTRLGRSIRATANHRFLTPQG | | ANDIIAHN | |
| | WKRVDELQPGDYLALPRRIPTASTPTL | | | |
| mNpu- | CLSYDTEILTVEYGILPIGKIVEKRIECT | 31 | VKVIGRRSLGVQRIFDIGLPQY | 32 |
| DnaE | VYSVDNNGNIYTQPVAQWHDRGEQE | | HNFLLANGAIAAN | |
| | VFEYCLEDGSLIRATKDHKFMTVDGQ | | | |
| | MMPIDEIFERELDLMRVDNLPN | | | |
| Cfa | CLSYDTEILTVEYGFLPIGKIVEERIECT | 33 | VKIISRKSLGTQNVYDIGVEKDH | 34 |
| | VYTVDKNGFVYTQPIAQWHNRGEQE | | NFLLKNGLVASN | |
| | VFEYCLEDGSIIRATKDHKFMTTDGQ | | | |
| | MLPIDEIFERGLDLKQVDGLP | | | |
| DnaE | CLSYDTEILTVEYGFLPIGKIVEERIECT | 35 | KRTADGSEFESPKKKRKVKIIS | 36 |
| | VYTVDKNGFVYTQPIAQWHNRGEQE | | RKSLGTQNVYDIGVEKDHNFLL | |
| | VFEYCLEDGSIIRATKDHKFMTTDGQ | | KNGLVASN | |
| | MLPIDEIFERGLDLKQVDGLP | | | |
| Ssp | CLSFGTEILTVEYGPLPIGKIVSEEINCS | 37 | VKVIGRRSLGVQRIFDIGLPQD | 38 |
| DnaE | VYSVDPEGRVYTQAIAQWHDRGEQE | | HNFLLANGAIAANC | |
| | VLEYELEDGSVIRATSDHRFLTTDYQL | | | |
| | LAIEEIFARQLDLLTLENIKQTEEALDN | | | |
| | HRLPFPLLDAGTIK | | | |
| DnaE | CLSYDTEILTVEYGFLPIGKIVEERIECT | 39 | GLPVKIISRKSLGTQNVYDIGVE | 40 |
| | VYTVDKNGFVYTQPIAQWHNRGEQE | | KDHNFLLKNGLVASN | |
| | VFEYCLEDGSIIRATKDHKFMTTDGQ | | | |
| | MLPIDEIFERGLDLQVD | | | |

The Npu N-intein of SEQ ID NO: 27 may be encoded by a polynucleotide having the DNA sequence of SEQ ID NO: 41, as is shown below.

(SEQ ID NO: 41)
TGCCTGAGCTACGAGACCGAGATCCTGACCGTGGAGTACGGCCTGCTGC
CCATCGGCAAGATCGTGGAGAAGAGAATCGAGTGCACCGTGTACAGCGT
GGACAACAACGGCAACATCTACACCCAGCCCGTGGCCCAGTGGCACGAC
AGAGGCGAGCAGGAGGTGTTCGAGTACTGCCTGGAGGACGGCAGCCTGA
TCAGAGCCACCAAGGACCACAAGTTCATGACCGTGGACGGCCAGATGCT
GCCCATCGACGAGATCTTCGAGAGAGAGCTGGACCTGATGAGAGTGGAC
AACCTGCCCAAC

The Npu C-intein of SEQ ID NO: 28 may be encoded by a polynucleotide having the DNA sequence of SEQ ID NO: 42, as is shown below.

(SEQ ID NO: 42)
ATCAAGATCGCCACAAGAAAGTACCTGGGCAAGCAGAACGTGTACGACA
TCGGCGTGGAGAGAGACCACAACTTCGCCCTGAAGAACGGCTTCATCGC
CAGCAAT

A split intein of the disclosure (i.e., the N-intein and C-intein) can include nucleophile amino acid at or near its N- or C-terminus that is capable of performing the trans-splicing reaction. In some embodiments, the nucleophile amino acid is selected from serine, threonine, cysteine, or alanine.

In some embodiments, the first vector and/or the second vector further include one or more additional regulatory sequences, such as, e.g., a Woodchuck Hepatitis Virus Posttranscriptional Regulatory Element (WPRE), an enhancer sequence, a poly(A) sequence, a terminator sequence, or a degradation signal, among others.

In some embodiments, the split intein system described herein includes a ligand-dependent intein, which performs protein splicing upon contact with a ligand (e.g., small molecules such as 4-hydroxytamoxifen, peptides, proteins, polynucleotides, amino acids, nucleotides, etc.). Various ligand-dependent inteins are described in US 2014/0065711, the disclosure of which is incorporated by reference herein as it relates to ligand-dependent inteins.

The present disclosure provides vectors containing one or more degradation signals within the intein (e.g., N-intein or C-intein) polypeptide(s) that mediate protein degradation by ubiquitin-proteasome system and/or autophagy-lysosome pathways. Such sequences may be incorporated into the vector systems of the disclosure to avoid or reduce accumulation of excised intein proteins within target cells.

Exemplary degradation signals include N-degrons and C-degrons, which are peptide sequences containing motifs containing lysine residues capable of polyubiquitylation and subsequent targeting for degradation. In some embodiments, degrons are degradation signals located within a protein sequence (e.g., an intein sequence) that is not at the N-terminus nor the C-terminus of the protein sequence. In some embodiments, the N-intein protein includes one or more (e.g., 2, 3, 4, 5, or more) degrons. In some embodiments, the C-intein protein includes one or more (e.g., 2, 3, 4, 5, or more) degrons. In some embodiments, the degron is a CL1 degron, which is a C-terminal destabilizing peptide that shares structural similarity with misfolded proteins and is recognized by the ubiquitination system. In some embodiments, the degron is a PB29, SMN, CIITA, or ODC degron. Such degradation signals are described in WO 2016/13932, which is incorporated by reference herein as it relates to degradation signals. Another example of a degradation signal includes the E. coli dihydrofolate reductase (ecDHFR)-derived degron, as is described in WO 2020/079034 (incorporated by reference herein). Additional degradation signals include FKBP12 degradation domains (Banaszynski et al., Cell 126:995-1004, 2006), PEST degradation domains (Rechsteiner and Rogers, Trends Biochem Sci. 21:267-271, 1996), UbR tag ubiquitination signals (Chassin et al., Nat Commun. 10:2013, 2019), and destabilized mutations of human ELRBD (Miyazaki et al., J. Am. Chem. Soc., 134:3942-3945, 2012).

### Vectors for the Expression of Stereocilin

In addition to achieving high rates of transcription and translation, stable expression of an exogenous gene in a mammalian cell can be achieved by integration of the polynucleotide containing the gene into the nuclear genome of the mammalian cell. A variety of vectors for the delivery and integration of polynucleotides encoding stereocilin into the nuclear DNA of a mammalian cell have been developed. Examples of expression vectors are described in, e.g., Gellissen, Production of Recombinant Proteins: Novel Microbial and Eukaryotic Expression Systems (John Wiley & Sons, Marblehead, M A, 2006). Expression vectors for use in the compositions and methods described herein contain an OCM promoter (e.g., a polynucleotide having at least 85% sequence identity (e.g., 86%, 87%, 88%, 89%, 90%, 91%, 92%, 93%, 94%, 95%, 96%, 97%, 98%, 99%, or more, sequence identity) to any one of the promoter sequences listed in Table 3 (e.g., any one of SEQ ID NOs: 1-3)) operably linked to a polynucleotide sequence that encodes a portion of a stereocilin protein (e.g., a portion of SEQ ID NO: 4 or SEQ ID NO: 5), as well as, e.g., additional sequence elements used for the expression of these agents and/or the integration of these polynucleotide sequences into the genome of a mammalian cell. Vectors that can contain an OCM-specific promoter operably linked to a transgene encoding a portion of a stereocilin protein include plasmids (e.g., circular DNA molecules that can autonomously replicate inside a cell), cosmids (e.g., pWE or sCos vectors), artificial chromosomes (e.g., a human artificial chromosome (HAC), a yeast artificial chromosome (YAC), a bacterial artificial chromosome (BAC), or a P1-derived artificial chromosome (PAC)), and viral vectors. Certain vectors that can be used for the expression of stereocilin include plasmids that contain regulatory sequences, such as enhancer regions, which direct gene transcription. Other useful vectors for expression of a stereocilin protein contain polynucleotide sequences that enhance the rate of translation of this gene or improve the stability or nuclear export of the mRNA that results from gene transcription. These sequence elements include, e.g., 5′ and 3′ untranslated regions, an internal ribosomal entry site (IRES), and polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

### Viral Vectors for Polynucleotide Delivery

Viral genomes provide a rich source of vectors that can be used for the efficient delivery of STRC into the genome of a target cell (e.g., a mammalian cell, such as a human cell). Viral genomes are particularly useful vectors for gene delivery because the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors include a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology, Third Edition (Lippincott-Raven, Philadelphia, 1996)). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, U.S. Pat. No. 5,801,030, the disclosure of which is incorporated herein by reference as it pertains to viral vectors for use in gene therapy.

### AAV Vectors for Polynucleotide Delivery

In some embodiments, the polynucleotides of the compositions and methods described herein are incorporated into rAAV vectors and/or virions in order to facilitate their introduction into a cell. rAAV vectors useful in the compositions and methods described herein are recombinant polynucleotide constructs that include (1) an OCM promoter described herein (e.g., a polynucleotide having at least 85% sequence identity (e.g., 86%, 87%, 88%, 89%, 90%, 91%, 92%, 93%, 94%, 95%, 96%, 97%, 98%, 99%, or more, sequence identity) to any one of the promoter sequences listed in Table 3 (e.g., any one of SEQ ID NOs: 1-3)), (2) a heterologous sequence to be expressed (e.g., a polynucleotide encoding an N-terminal portion or C-terminal portion of a stereocilin protein), and (3) viral sequences that facilitate integration and expression of the heterologous genes. The viral sequences may include those sequences of AAV that are required in cis for replication and packaging (e.g., functional ITRs) of the DNA into a virion. In typical applications, the transgene encodes a wild-type form of a protein (e.g., stereocilin) that is mutated in subjects with forms of hereditary hearing loss that may be useful for improving hearing in subjects carrying mutations that have been associated with hearing loss or deafness (e.g., DFNB16). Such rAAV vectors may also contain marker or reporter genes. Useful rAAV vectors have one or more of the AAV WT genes deleted in whole or in part but retain functional flanking ITR sequences. The AAV ITRs may be of any serotype suitable for a particular application. For use in the methods and compositions described herein, the ITRs can be AAV2 ITRs. Methods for using rAAV vectors are described, for example, in Tal et al., J. Biomed. Sci. 7:279 (2000), and Monahan and Samulski, Gene Delivery 7:24 (2000), the disclosures of each of which are incorporated herein by reference as they pertain to AAV vectors for gene delivery.

The polynucleotides and vectors described herein (e.g., an OCM promoter operably linked to a polynucleotide encoding a N-terminal, or, in some embodiments, a C-terminal portion of the stereocilin protein) can be incorporated into a rAAV virion in order to facilitate introduction of the polynucleotide or vector into a cell. The capsid proteins of AAV compose the exterior, non-nucleic acid portion of the virion and are encoded by the AAV cap gene. The cap gene encodes three viral coat proteins, VP1, VP2 and VP3, which are required for virion assembly. The construction of rAAV virions has been described, for instance, in U.S. Pat. Nos. 5,173,414; 5,139,941; 5,863,541; 5,869,305; 6,057,152; and 6,376,237; as well as in Rabinowitz et al., J. Virol. 76:791 (2002) and Bowles et al., J. Virol. 77:423 (2003), the disclosures of each of which are incorporated herein by reference as they pertain to AAV vectors for gene delivery.

rAAV virions useful in conjunction with the compositions and methods described herein include those derived from a variety of AAV serotypes including AAV 1, 2, 3, 4, 5, 6, 7, 8, 9, 10, rh10, rh39, rh43, rh74, Anc80, Anc80L65, DJ/8, DJ/9, 7m8, PHP.B, PHP.eb, and PHP.S. For targeting hair cells, AAV1, AAV2, AAV2quad(Y-F), AAV6, AAV8, AAV9, Anc80, Anc80L65, DJ/9, 7m8, and PHP.B may be particularly useful. Serotypes evolved for transduction of the retina may also be used in the methods and compositions described herein. Construction and use of AAV vectors and AAV proteins of different serotypes are described, for instance, in Chao et al., Mol. Ther. 2:619 (2000); Davidson et al., Proc. Natl. Acad. Sci. USA 97:3428 (2000); Xiao et al., J. Virol. 72:2224 (1998); Halbert et al., J. Virol. 74:1524 (2000); Halbert et al., J. Virol. 75:6615 (2001); and Auricchio et al., Hum. Molec. Genet. 10:3075 (2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype (e.g., AAV9) pseudotyped with a capsid gene derived from a serotype other than the given serotype (e.g., AAV1, AAV2, AAV2quad(Y-F), AAV3, AAV4, AAV5, AAV6, AAV7, AAV8, etc.). Techniques involving the construction and use of pseudotyped rAAV virions are known in the art and are described, for instance, in Duan et al., J. Virol. 75:7662 (2001); Halbert et al., J. Virol. 74:1524 (2000); Zolotukhin et al., Methods, 28:158 (2002); and Auricchio et al., Hum. Molec. Genet. 10:3075 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635 (2000). Other rAAV virions that can be used in methods described herein include those capsid hybrids that are generated by molecular breeding of viruses as well as by exon shuffling. See, e.g., Soong et al., Nat. Genet., 25:436 (2000) and Kolman and Stemmer, Nat. Biotechnol. 19:423 (2001).

### Pharmaceutical Compositions

The nucleic acid vectors described herein may be incorporated into a vehicle for administration into a patient, such as a human patient suffering from sensorineural hearing loss. Pharmaceutical compositions containing vectors, such as viral vectors, that contain a polynucleotide encoding a portion of a stereocilin protein can be prepared using methods known in the art. For example, such compositions can be prepared using, e.g., physiologically acceptable carriers, excipients, or stabilizers (Remington: The Science and Practice of Pharmacology 22nd edition, Allen, L. Ed. (2013); incorporated herein by reference), and in a desired form, e.g., in the form of lyophilized formulations or aqueous solutions.

Mixtures of nucleic acid vectors (e.g., viral vectors) described herein may be prepared in water suitably mixed with one or more excipients, carriers, or diluents. Dispersions may also be prepared in glycerol, liquid polyethylene glycols, and mixtures thereof and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (described in U.S. Pat. No. 5,466,468, the disclosure of which is incorporated herein by reference). In any case the formulation may be sterile and may be fluid to the extent that easy syringability exists. Formulations may be stable under the conditions of manufacture and storage and may be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For example, a solution containing a pharmaceutical composition described herein may be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the subject being treated. For local administration to the inner ear, the composition may be formulated to contain a synthetic perilymph solution. An exemplary synthetic perilymph solution includes 20-200 mM NaCl, 1-5 mM KCl, 0.1-10 mM CaCl₂), 1-10 mM glucose, and 2-50 mM HEPEs, with a pH between about 6 and 9 and an osmolality of about 300 mOsm/kg. The person responsible for administration will, in any event, determine the appropriate dose for the individual subject. Moreover, for human administration, preparations may meet sterility, pyrogenicity, general safety, and purity standards as required by FDA Office of Biologics standards.

### Methods of Treatment

The compositions described herein may be administered to a subject having or at risk of developing sensorineural hearing loss by a variety of routes, such as local administration to the inner ear (e.g., administration into the perilymph or endolymph, such as to or through the oval window, round window, or semicircular canal (e.g., horizontal canal), or by transtympanic or intratympanic injection, e.g., administration to an OHC), intravenous, parenteral, intradermal, transdermal, intramuscular, intranasal, subcutaneous, percutaneous, intratracheal, intraperitoneal, intraarterial, intravascular, inhalation, perfusion, lavage, and oral administration. The most suitable route for administration in any given case will depend on the particular composition administered, the patient, pharmaceutical formulation methods, administration methods (e.g., administration time and administration route), the patient's age, body weight, sex, severity of the disease being treated, the patient's diet, and the patient's excretion rate. Compositions may be administered once, or more than once (e.g., once annually, twice annually, three times annually, bi-monthly, or monthly). In some embodiments, the first and second nucleic acid vectors are administered simultaneously (e.g., in one composition). In some embodiments, the first and second nucleic acid vectors are administered sequentially (e.g., the second nucleic acid vector is administered immediately after the first nucleic acid vector, or 5 minutes, 10 minutes, 15 minutes, 20 minutes, 25 minutes, 30 minutes, 45 minutes, 1 hour, 2 hours, 8 hours, 12 hours, 1 day, 2 days, 7 days, two weeks, 1 month or more after the first nucleic acid vector). The first and second nucleic acid vector can have the same capsid or different capsids (e.g., AAV capsids).

Subjects that may be treated as described herein are subjects having or at risk of developing sensorineural hearing loss. The compositions and methods described herein can be used to treat subjects having a mutation in STRC (e.g., a mutation that reduces STRC function or expression, or an STRC mutation associated with sensorineural hearing loss, such as subjects having DFNB16), subjects having a family history of autosomal recessive sensorineural hearing loss or deafness (e.g., a family history of STRC-related hearing loss), or subjects whose STRC mutational status and/or STRC activity level is unknown. The methods described herein may include a step of screening a subject for a mutation in STRC prior to treatment with or administration of the compositions described herein. A subject can be screened for an STRC mutation using standard methods known to those of skill in the art (e.g., genetic testing). The methods described herein may also include a step of assessing hearing in a subject prior to treatment with or administration of the compositions described herein. Hearing can be assessed using standard tests, such as audiometry, auditory brainstem response (ABR), electrocochleography (ECOG), and otoacoustic emissions. The compositions and methods described herein may also be administered as a preventative treatment to patients at risk of developing hearing loss or auditory neuropathy, e.g., patients who have a family history of inherited hearing loss or patients carrying an STRC mutation who do not yet exhibit hearing loss or impairment.

Treatment may include administration of a composition containing the nucleic acid vectors (e.g., AAV viral vectors) described herein in various unit doses. Each unit dose will ordinarily contain a predetermined quantity of the therapeutic composition. The quantity to be administered, and the particular route of administration and formulation, are within the skill of those in the clinical arts. A unit dose need not be administered as a single injection but may comprise continuous infusion over a set period of time. Dosing may be performed using a syringe pump to control infusion rate in order to minimize damage to the inner ear (e.g., the cochlea). In cases in which the nucleic acid vectors are AAV vectors (e.g., AAV1, AAV2, AAV2quad(Y-F), AAV3, AAV4, AAV5, AAV6, AAV7, AAV8, AAV9, AAV10, rh10, rh39, rh43, rh74, Anc80, Anc80L65, DJ/8, DJ/9, 7m8, PHP.B, PHP.eb, or PHP.S vectors), the viral vectors may be administered to the patient at a dose of, for example, from about 1×10⁹vector genomes (VG)/mL to about 1×10¹⁶ VG/mL (e.g., 1×10⁹ VG/mL, 2×10⁹ VG/mL, 3×10⁹ VG/mL, 4×10⁹ VG/mL, 5×10⁹ VG/mL, 6×10⁹ VG/mL, 7×10⁹ VG/mL, 8×10⁹ VG/mL, 9×10⁹ VG/mL, 1×10¹⁰ VG/mL, 2×10¹⁰ VG/mL, 3×10¹⁰ VG/mL, 4×10¹⁰ VG/mL, 5×10¹⁰ VG/mL, 6×10¹⁰ VG/mL, 7×10¹⁰ VG/mL, 8×10¹⁰ VG/mL, 9×10¹⁰ VG/mL, 1×10¹¹ VG/mL, 2×10¹¹ VG/mL, 3×10¹¹ VG/mL, 4×10¹¹ VG/mL, 5×10¹¹ VG/mL, 6×10¹¹ VG/mL, 7×10¹¹ VG/mL, 8×10¹¹ VG/mL, 9×10¹¹ VG/mL, 1×10¹² VG/mL, 2×10¹² VG/mL, 3×10¹² VG/mL, 4×10¹² VG/mL, 5×10¹² VG/mL, 6×10¹² VG/mL, 7×10¹² VG/mL, 8×10¹² VG/mL, 9×10¹² VG/mL, 1×10¹³ VG/mL, 2×10¹³ VG/mL, 3×10¹³ VG/mL, 4×10¹³ VG/mL, 5×10¹³ VG/mL, 6×10¹³ VG/mL, 7×10¹³ VG/mL, 8×10¹³ VG/mL, 9×10¹³ VG/mL, 1×10¹⁴ VG/mL, 2×10¹⁴ VG/mL, 3×10¹⁴ VG/mL, 4×10¹⁴ VG/mL, 5×10¹⁴ VG/mL, 6×10¹⁴ VG/mL, 7×10¹⁴ VG/mL, 8×10¹⁴ VG/mL, 9×10¹⁴ VG/mL, 1×10¹⁵ VG/mL, 2×10¹⁵ VG/mL, 3×10¹⁵ VG/mL, 4×10¹⁵ VG/mL, 5×10¹⁵ VG/mL, 6×10¹⁵ VG/mL, 7×10¹⁵ VG/mL, 8×10¹⁵ VG/mL, 9×10¹⁵ VG/mL, or 1×10¹⁶ VG/mL) in a volume of 1 μL to 200 μL (e.g., 1, 2, 3, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or 200 μL). The AAV vectors may be administered to the subject at a dose of about 1×10⁷ VG/ear to about 2×10¹⁵ VG/ear (e.g., 1×10⁷ VG/ear, 2×10⁷ VG/ear, 3×10⁷ VG/ear, 4×10⁷ VG/ear, 5×10⁷ VG/ear, 6×10⁷ VG/ear, 7×10⁷ VG/ear, 8×10⁷ VG/ear, 9×10⁷ VG/ear, 1×10⁸ VG/ear, 2×10⁸ VG/ear, 3×10⁸ VG/ear, 4×10⁸ VG/ear, 5×10⁸ VG/ear, 6×10⁸ VG/ear, 7×10⁸ VG/ear, 8×10⁸ VG/ear, 9×10⁸ VG/ear, 1×10⁹ VG/ear, 2×10⁹ VG/ear, 3×10⁹ VG/ear, 4×10⁹ VG/ear, 5×10⁹ VG/ear, 6×10⁹ VG/ear, 7×10⁹ VG/ear, 8×10⁹ VG/ear, 9×10⁹ VG/ear, 1×10¹⁰VG/ear, 2×10¹⁰VG/ear, 3×10¹⁰ VG/ear, 4×10¹⁰ VG/ear, 5×10¹⁰VG/ear, 6×10¹⁰ VG/ear, 7×10¹⁰ VG/ear, 8×10¹⁰ VG/ear, 9×10¹⁰ VG/ear, 1×10¹¹ VG/ear, 2×10¹¹ VG/ear, 3×10¹ VG/ear, 4×10¹¹ VG/ear, 5×10¹¹ VG/ear, 6×10¹¹ VG/ear, 7×10¹¹ VG/ear, 8×10¹¹ VG/ear, 9×10¹¹ VG/ear, 1×10¹² VG/ear, 2×10¹² VG/ear, 3×10¹² VG/ear, 4×10¹² VG/ear, 5×10¹² VG/ear, 6×10¹² VG/ear, 7×10¹² VG/ear, 8×10¹² VG/ear, 9×10¹² VG/ear, 1×10¹³ VG/ear, 2×10¹³ VG/ear, 3×10¹³ VG/ear, 4×10¹³ VG/ear, 5×10¹³ VG/ear, 6×10¹³ VG/ear, 7×10¹³ VG/ear, 8×10¹³ VG/ear, 9×10¹³ VG/ear, 1×10¹⁴ VG/ear, 2×10¹⁴ VG/ear, 3×10¹⁴ VG/ear, 4×10¹⁴ VG/ear, 5×10¹⁴ VG/ear, 6×10¹⁴ VG/ear, 7×10¹⁴ VG/ear, 8×10¹⁴ VG/ear, 9×10¹⁴ VG/ear, 1×10¹⁵ VG/ear, or 2×10¹⁵ VG/ear).

The compositions described herein are administered in an amount sufficient to improve hearing, increase expression of a stereocilin protein (e.g., a WT stereocilin protein, such as a stereocilin protein having the sequence of SEQ ID NO: 4 or SEQ ID NO: 5, e.g., expression in a cochlear hair cell, e.g., an outer hair cell), increase stereocilin function, improve OHC structure, improve OHC function, prevent or reduce OHC damage or death, improve OHC hair bundle attachment to the tectorial membrane, or increase or improve OHC survival. Hearing may be evaluated using standard hearing tests (e.g., audiometry, ABR, electrocochleography (ECOG), and otoacoustic emissions) and may be improved by 5% or more (e.g., 5%, 10%, 15%, 20%, 30%, 40%, 50%, 60%, 70%, 80%, 90%, 100%, 125%, 150%, 200% or more) compared to hearing measurements obtained prior to treatment. In some embodiments, the compositions are administered in an amount sufficient to improve the subject's ability to understand speech. The compositions described herein may also be administered in an amount sufficient to slow or prevent the development or progression of sensorineural hearing loss (e.g., in subjects who carry a genetic mutation in the STRC gene that is associated with hearing loss or have a family history of hearing loss (e.g., autosomal recessive hearing loss) but do not exhibit hearing impairment, or in subjects exhibiting mild to moderate hearing loss). Stereocilin expression may be evaluated using immunohistochemistry, western blot analysis, quantitative real-time PCR, or other methods known in the art for detection protein or mRNA, and may be increased by 5% or more (e.g., 5%, 10%, 15%, 20%, 30%, 40%, 50%, 60%, 70%, 80%, 90%, 100%, 125%, 150%, 200% or more) compared to stereocilin expression prior to administration of the compositions described herein. OHC function or function of the stereocilin protein encoded by the nucleic acid vectors administered to the subject may be evaluated indirectly based on hearing tests, and may be increased by 5% or more (e.g., 5%, 10%, 15%, 20%, 30%, 40%, 50%, 60%, 70%, 80%, 90%, 100%, 125%, 150%, 200% or more) compared to OHC function or function of the protein prior to administration of the compositions described herein. These effects may occur, for example, within 1 week, 2 weeks, 3 weeks, 4 weeks, 5 weeks, 6 weeks, 7 weeks, 8 weeks, 9 weeks, 10 weeks, 15 weeks, 20 weeks, 25 weeks, or more, following administration of the compositions described herein. The patient may be evaluated 1 month, 2 months, 3 months, 4 months, 5 months, 6 months, or more following administration of the composition depending on the dose and route of administration used for treatment. Depending on the outcome of the evaluation, the patient may receive additional treatments.

### Kits

The compositions described herein can be provided in a kit for use in treating a subject with sensorineural hearing loss, such as sensorineural hearing loss associated with a mutation in the STRC gene. Compositions may include the polynucleotides described herein (e.g., a polynucleotide having at least 85% sequence identity (e.g., 86%, 87%, 88%, 89%, 90%, 91%, 92%, 93%, 94%, 95%, 96%, 97%, 98%, 99%, or more, sequence identity) to any one of the OCM promoter sequences listed in Table 3 (e.g., any one of SEQ ID NOs: 1-3) operably linked to a polynucleotide encoding an N-terminal portion of the stereocilin protein, and a polynucleotide encoding a C-terminal portion of the stereocilin protein) and nucleic acid vector systems (e.g., two-vector systems described herein) containing such polynucleotides. The nucleic acid vectors may be packaged in an AAV virus capsid (e.g., AAV1, AAV2, AAV2quad(Y-F), AAV6, AAV8, AAV9, Anc80, Anc80L65, DJ/9, 7m8, or PHP.B). The kit can further include a package insert that instructs a user of the kit, such as a physician, to perform the methods described herein. The kit may optionally include a syringe or other device for administering the composition.

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure.

### Example 1. OCM Promoter Sequence Induces Transgene Expression in OHCs in Murine Cochlea In Vivo

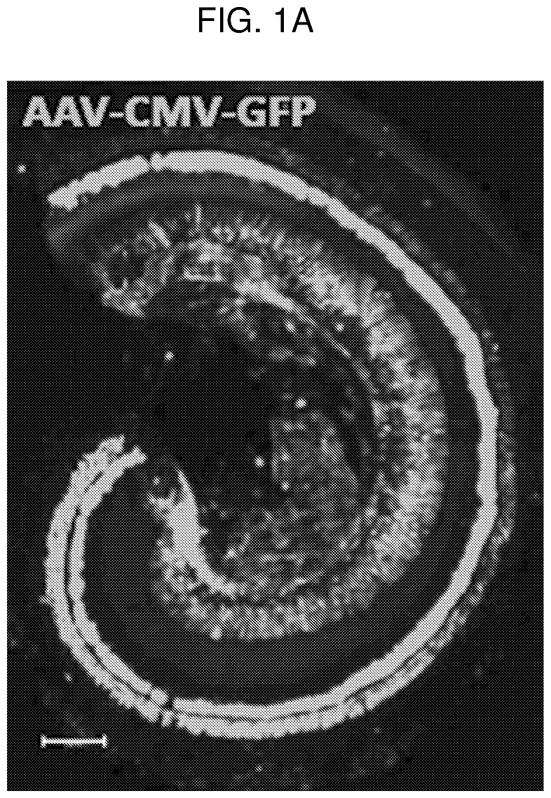

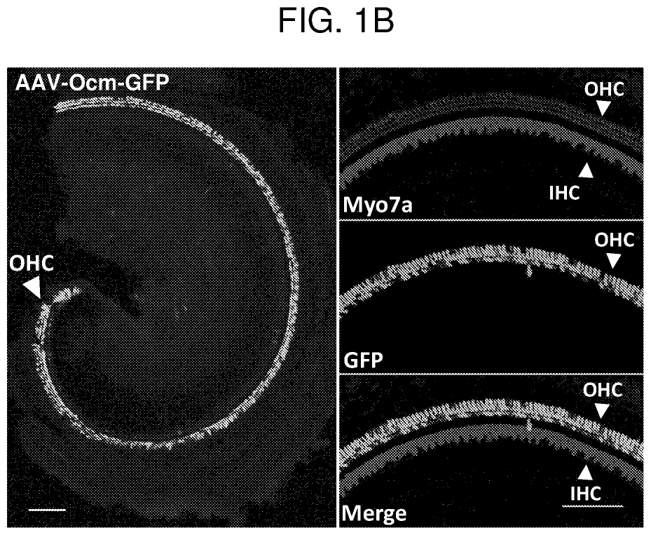

To determine the efficacy of the constructed OCM promoter (SEQ ID NO: 1) in inducing transgene expression in OHCs in vivo, mouse cochlea was transduced with either an AAV vector expressing GFP under the control of the cytomegalovirus (CMV) promoter, or an AAV vector expressing GFP under control of the OCM promoter. Specifically, AAV-OCM-GFP virus was infused via the posterior semicircular canal to two-day-old CBA/CaJ mice at a dose of 7.7E+9 vector genomes per ear. Mice recovered from surgery and were euthanized and perfused with 10% normal buffered formalin 19 days later. The inner ear temporal bone was harvested and decalcified in 8% EDTA for three days. The cochlea was dissected from the de-calcified temporal bone, immunostained with Myosin 7a (Myo7a) antibody to label all hair cells, and mounted on a slide for confocal imaging. Native GFP fluorescence is shown. Using a ubiquitous promoter, AAV-CMV-GFP induced GFP expression in many cell types within the cochlea including inner hair cells, outer hair cells, spiral ganglion neurons, mesenchymal cells, and glia (FIG. 1A). Using the outer hair cell-specific promoter, AAV-OCM (SEQ ID NO: 1)-GFP induced GFP expression exclusively in outer hair cells (FIG. 1B).

### Example 2. OCM Promoter-Driven GFP Expression is Enriched in Outer Hair Cells in the Organ of Corti of Non-Human Primates

To test the specificity of the OCM promoter of SEQ ID NO: 1 in non-human primates in vivo, non-human primate (Macaca fascicularis) ears were injected with an AAV vector including nuclear targeted H2B-eGFP operably linked to the OCM promoter of SEQ ID NO: 1. Adult non-human primates were injected with 40 μl of vector (3.41×10¹³ vg/ml) for the AAV vector expressing H2B-eGFP under control of the OCM promoter of SEQ ID NO: 1 via the round window membrane, a fenestration in the lateral semicircular canal allowed for efflux of perilymph during the procedure.

After four weeks in life animals were sacrificed and fixed in 10% NBF via cardiac perfusion, their temporal bones were harvested and kept in 10% NBF for additional 4-10 days. Ears were decalcified in formic acid (immunocal) for 6 days and paraffin embedded and sectioned in 5 μm slices.

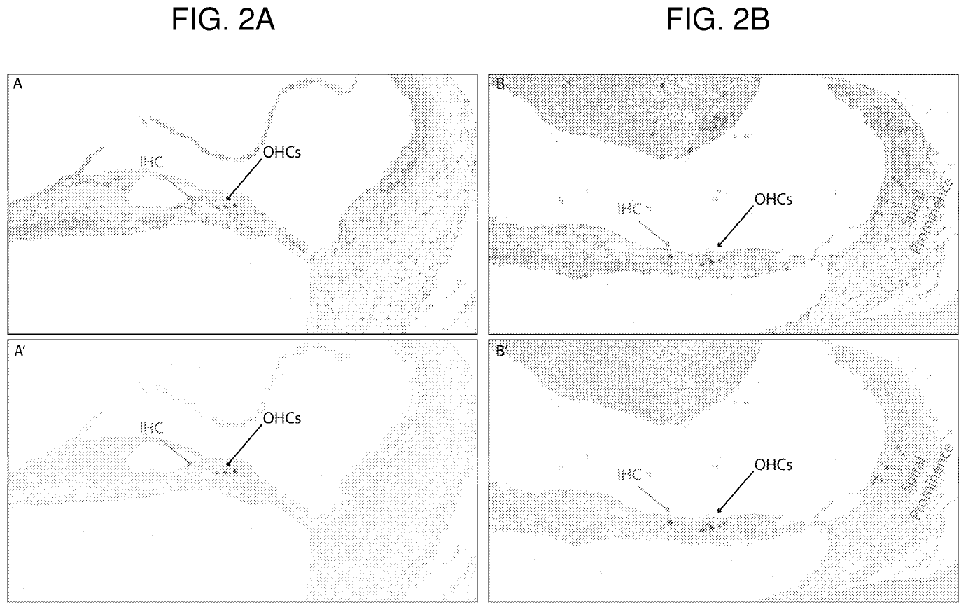

Sections were labeled with an antibody against GFP and stained with a secondary antibody conjugated to alkaline phosphatase; a red, chromatic staining was developed by the reaction of the fast red dye with the alkaline phosphatase of the secondary antibody. Sections were counterstained with Hematoxylin in blue to visualize all nuclei and imaged on a color camera at 20× magnification and converted to greyscale (FIGS. 2A-2B, upper images (panels A and B, respectively)—FIG. 2A is a micrograph of single paraffin section from a first animal and FIG. 2B is a micrograph of single paraffin section from a second animal). To visualize the red signal of the chromatic anti-GFP staining, all blue (Hematoxylin) color was extracted from the color images using Image processing software GIMP utilizing the select color tool, before converting to greyscale. Only nuclei with red signal nuclear H2B-GFP remained visible (FIGS. 2A-2B, lower images (panels A′ and B′, respectively)). Scale bars represent 100 μm. Inner hair cells (IHCs) and outer hair cells (OHCs) are highlighted for orientation.

### Example 3. An Anc80-CMV-mStrc Overlapping Dual Vector System Rescued Hearing in Stereocilin Deficient Mice

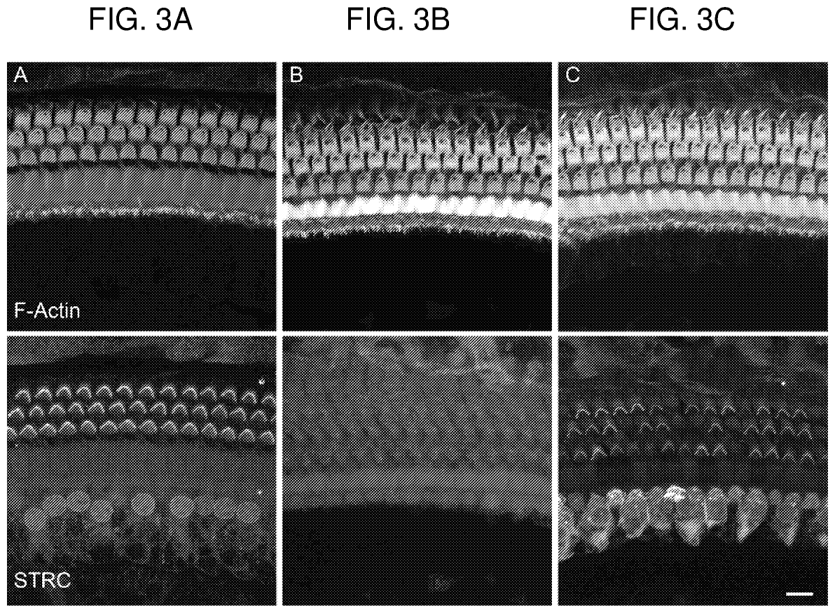

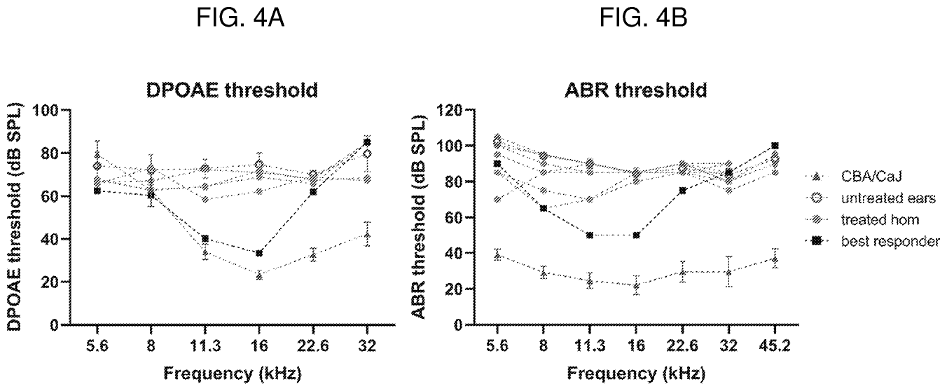

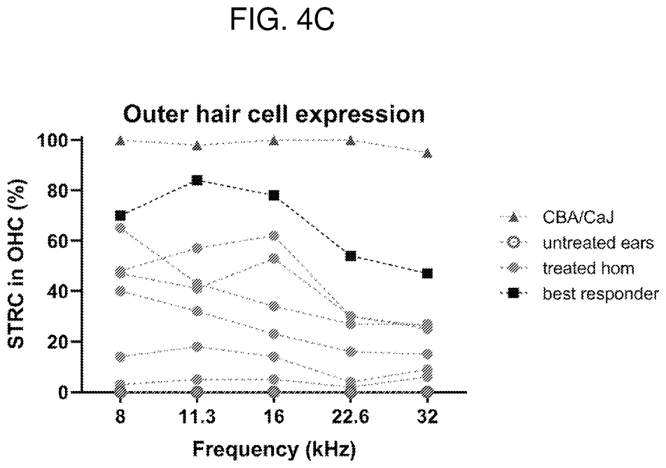

CRISPR-Cas9 technology was used to generate stereocilin deficient mice in the CBA/CaJ background strain by creating a frameshift at base pair position 232 of STRC. Wild type animals of the CBA/CaJ background strain showed distinct stereocilin antibody staining at the tips of the outer hair cell (OHC) stereocilia (FIG. 3A, bottom panel), while 232 bp Strc-KO animals lacked the signal for the antibody (FIG. 3B, bottom panel). Murine STRC was encapsulated in dual Anc80 vectors, where the first vector carried a CMV promoter and nucleotides 1-3200 of the murine STRC cDNA the second carried amino acids 2201-5430, creating a 1000 bp overlap between the two halves of the full-length cDNA. After delivery of both vectors at concentration of 1E10 vg/ear into the cochlea of early postnatal 232 bp Strc-KO mice via their posterior semicircular canal, de-novo stereocilin protein expression could be observed at the tips of the OHC and in the body of inner hair cells of the organ of Corti in treated 232 bp Strc-KO mice (FIG. 3C, bottom panel). Distortion product otoacoustic emissions (DPOAE) were evaluated as a direct readout of OHC function and auditory brainstem response (ABR) was measured as a measure of an intact ascending auditory pathway four weeks after treatment with Anc80-CMV-mStrc (overlap). Untreated contralateral ears in 232 bp Strc-KO animals (“untreated ears”) showed near absent DPOAEs and highly elevated ABR thresholds indicative of loss of OHC function (FIGS. 4A-4B, open circles), while treated 232 bp Strc-KO animals (“treated hom”) showed recovery of hearing thresholds (FIGS. 4A-4B, filled circles). The best responder (FIGS. 4A-4B, black squares) of the treated 232 bp Strc-KO animals showed close to wild type (“CBA/CaJ”) (FIGS. 4A-4B, triangles) hearing thresholds. A high fraction of OHCs of 232 bp Strc-KO mice expressing stereocilin after treatment with AAV-Anc80-CMV-mStrc was found to promote hearing recovery (FIG. 4C).

### Example 4. A Two-Vector Split Intein System Reconstituted Full-Length Stereocilin In Vitro

To generate experimental plasmids, DNA encoding amino acids 1-746 of stereocilin (“N-Strc”) was genetically fused with DNA encoding the Npu N-intein fragment (SEQ ID NO: 41, which encodes the Npu N-intein of SEQ ID NO: 27) and cloned into a plasmid containing the constitutively active CMV promoter to generate CMV.N-Strc-N-Npu. DNA encoding amino acids 747-1809 of stereocilin (“C-Strc”) was genetically fused downstream of DNA encoding the Npu C-intein fragment (SEQ ID NO: 42, which encodes the Npu C-intein of SEQ ID NO: 28) and cloned into a plasmid containing the CMV promoter to produce CMV.C-Npu-C-Strc. As a control, the full-length stereocilin coding sequence (“FL-Strc”) was also cloned into a CMV plasmid to generate CMV.FL-Strc. CMV.GFP was used as a negative control.

HEK293T cells were transfected with either control plasmids or a combination of N-Strc and C-Strc plasmids using the Lipofectamine 3000 kit (Life Technologies) and were incubated under standard cell culture conditions for three days. Cell cultures were rinsed with PBS and cells were lysed to extract protein. Protein lysate concentrations were measured using the BCA assay, and a constant mass of protein was loaded for Western blotting using antibodies against beta actin and stereocilin. Densitometry measurements of the protein band intensities was used to determine the relative amount of full-length stereocilin from the sample.

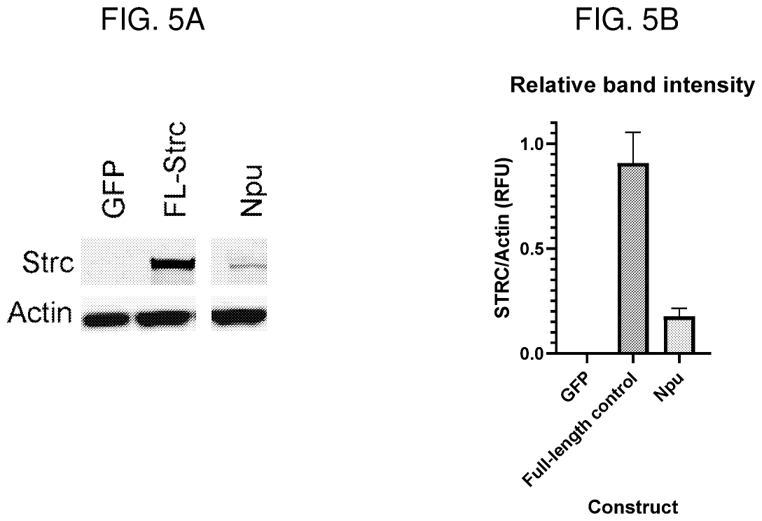

As shown in FIGS. 5A-5B, the tested intein designs produced a full-length stereocilin band.

### Example 5. AAV Dual Vector Systems for OCM Promoter-Driven Stereocilin Expression

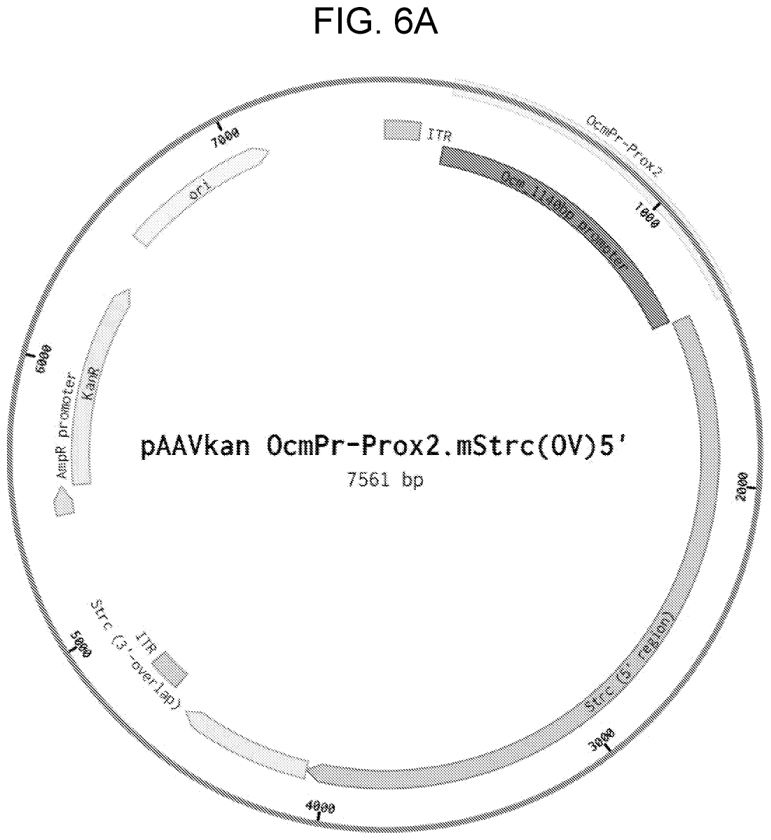

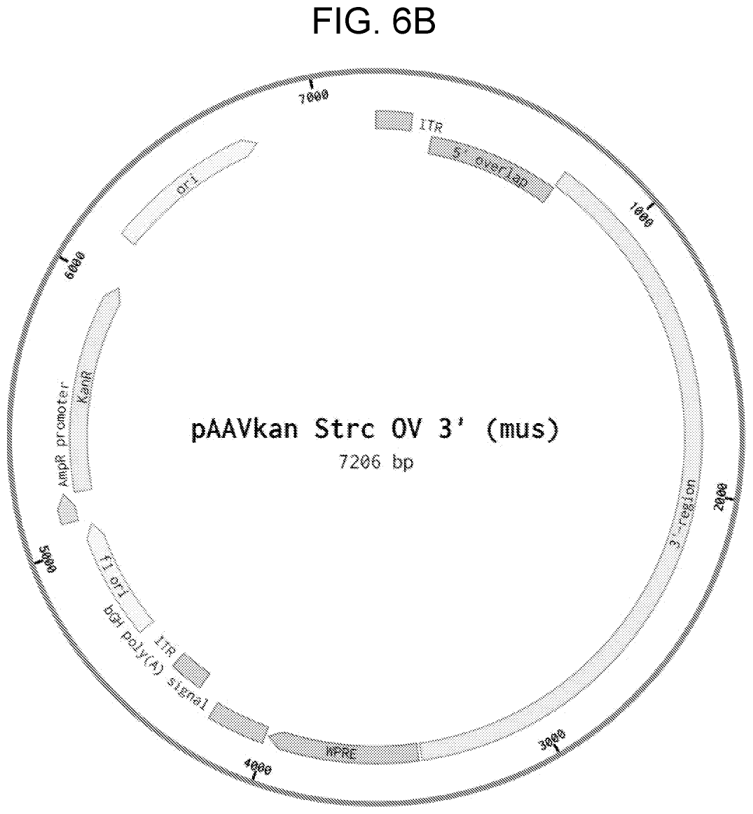

An AAV dual vector system using an overlap of stereocilin coding sequences for homologous recombination was designed using two plasmids. The first plasmid contained the murine OCM promoter of SEQ ID NO: 1 operably linked to an N-terminal portion of murine STRC encoded by the first 3200 nucleotides of the coding sequence and flanked by 5′ and 3′ ITR sequences (plasmid P959; SEQ ID NO: 43; FIG. 6A). The second plasmid (P724; SEQ ID NO: 44; FIG. 6B) contained, in 5′ to 3′ order, a 500 nucleotide overlap of the STRC sequence encoded by the first plasmid, followed by 2730 nucleotides encoding the remaining C-terminal portion of the murine STRC, a Woodchuck Posttranscriptional Regulatory Element (WPRE), and a bovine growth hormone (bGH) polyadenylation sequence. These elements in the second plasmid were also flanked by 5′ and 3′ ITR sequences.

AAV viral vectors are synthesized by transfecting HEK293T cells with one of these plasmids together with a rep/cap containing plasmid and an adenoviral helper plasmid using standard protocols. Plasmids are packaged into the AAV8 serotype vector using standard methods and obtained from a commercial vendor. The cell culture medium and the cells are subsequently collected to extract and purify the AAV. AAV from the cells is released from cells through three cycles of freeze thaw, and the cell culture medium is collected to obtain secreted AAV. AAV from the cell culture medium is concentrated by adding PEG8000 to the solution, incubating at 4° C., and centrifuging to collect the AAV particles. All AAV is passed through iodixanol density gradient centrifugation to purify the AAV particles, and the buffer is exchanged to PBS with 0.01% pluronic F68 by passing the purified AAV and the buffer over a centrifugation column with a 100 kDa molecular weight cutoff. The resulting AAV vectors from each of the two plasmids are used in combination by administration into the ears of mice (e.g., local administration to the inner ear).

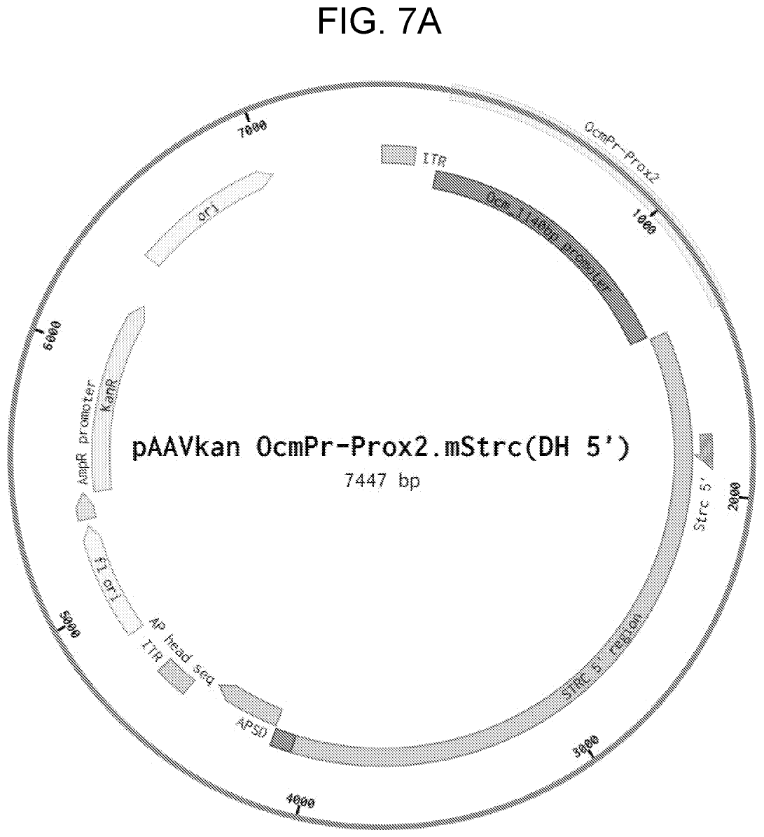

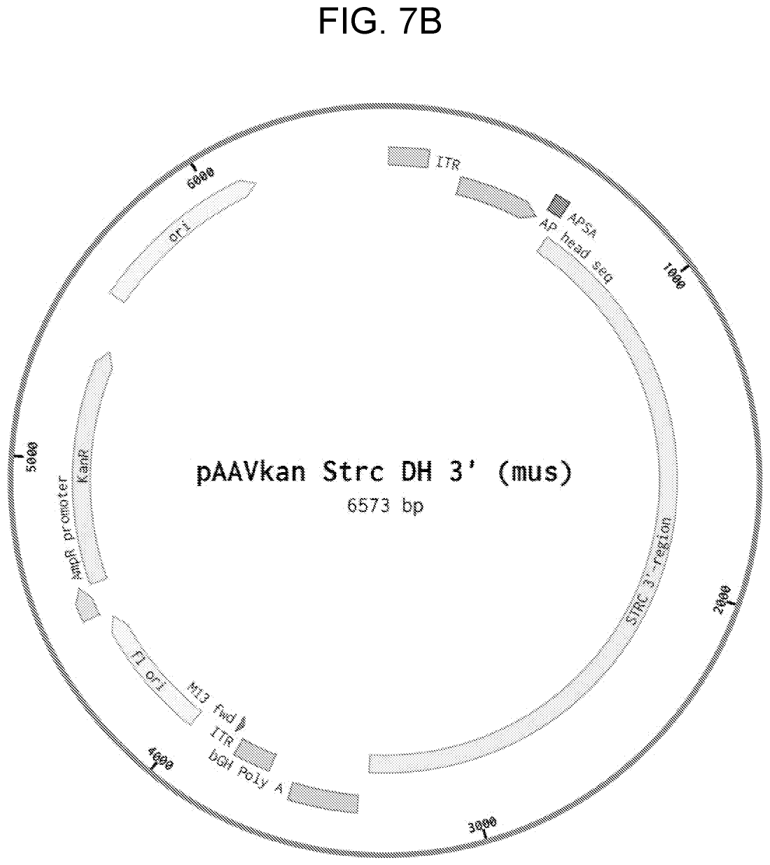

An alternative design of an AAV dual vector system utilized a first plasmid containing, in 5′ to 3′ order, a 5′ ITR sequence, the murine OCM promoter of SEQ ID NO: 1 operably linked to 2700 nucleotides encoding an N-terminal portion of murine stereocilin, an AP splice donor, an AP head sequence and a 3′ ITR sequence (P960; SEQ ID NO: 45; FIG. 7A). The second plasmid in this system contained, in 5′ to 3′ order, a 5′ ITR sequence, an AP head sequence homologous to the one in the first plasmid, an AP splice acceptor, the 2730 nucleotides encoding the remaining C-terminal portion of murine stereocilin, a bGH poly-A sequence and a 3′ ITR (P726; SEQ ID NO: 46; FIG. 7B). AAV viral vectors incorporating portions of these two plasmids are synthesized as described above.

### Example 6. Administration of a Composition Containing a Two-Vector System Containing an OCM Promoter Operably Linked to a Stereocilin Coding Sequence to a Subject with Sensorineural Hearing Loss

According to the methods disclosed herein, a physician of skill in the art can treat a patient, such as a human patient, with sensorineural hearing loss (e.g., sensorineural hearing loss associated with a mutation in STRC, such as DFNB16) so as to improve or restore hearing. To this end, a physician of skill in the art can administer to the human patient a composition containing a two-vector nucleic acid expression system, such as system that utilizes two AAV vectors (e.g., AAV1, AAV2, AAV2quad(Y-F), AAV6, AAV9, Anc80, Anc80L65, DJ/9, 7m8, or PHP.B vectors) that collectively include an OCM promoter (e.g., a polynucleotide having at least 85% sequence identity (e.g., 86%, 87%, 88%, 89%, 90%, 91%, 92%, 93%, 94%, 95%, 96%, 97%, 98%, 99%, or more, sequence identity) to the sequence of any one of SEQ ID NOs: 1-3) operably linked to an STRC transgene.

The two-vector system may be an overlapping dual vector system containing a first and second AAV vector. The overlapping dual vector system may include a first AAV vector that includes the OCM promoter operably linked to a polynucleotide encoding an N-terminal portion of the stereocilin protein (e.g., an N-terminal portion of SEQ ID NO: 4) and a second AAV vector that includes a polynucleotide encoding a C-terminal portion of the stereocilin protein, wherein the 3′ end of the stereocilin coding sequence in the first vector overlaps with the 5′ end of the stereocilin coding sequence in the second vector. In another example, the two-vector system may be a trans-splicing dual vector system containing a first and a second AAV vector. The trans-splicing dual vector system may include a first AAV vector that includes the OCM promoter operably linked to a polynucleotide encoding an N-terminal portion of the stereocilin protein (e.g., an N-terminal portion of SEQ ID NO: 4) and a splice donor signal sequence 3′ of the polynucleotide and a second AAV vector that includes a splice acceptor signal sequence 5′ of a polynucleotide encoding a C-terminal portion of the stereocilin protein. In another example, the two-vector system may be a dual hybrid vector system containing a first and second AAV vector. The dual hybrid vector system may include a first AAV vector that includes the OCM promoter operably linked to a polynucleotide encoding an N-terminal portion of the stereocilin protein (e.g., an N-terminal portion of SEQ ID NO: 4), a splice donor signal sequence 3′ of the polynucleotide, and a first recombinogenic region 3′ of the splice donor signal sequence, and a second AAV vector that includes a second recombinogenic region, a splice acceptor signal sequence 3′ of the recombinogenic region, and a polynucleotide encoding a C-terminal portion of the stereocilin protein 3′ of the splice acceptor signal sequence. In yet another example, the two-vector system may be a split intein trans-splicing system that includes a first AAV vector and a second AAV vector. The split intein trans-splicing two-vector system may include a first AAV vector that includes the OCM promoter operably linked to a polynucleotide encoding an N-terminal portion of the stereocilin protein (e.g., an N-terminal portion of SEQ ID NO: 4) and a polynucleotide encoding an N-terminal intein (N-intein) 3′ thereto, and a second AAV vector that includes the OCM promoter operably linked to a polynucleotide encoding a C-terminal intein (C-intein) and a polynucleotide encoding a C-terminal portion of the stereocilin protein 3′ thereto. The aforementioned two-vector systems may additionally include regulatory sequences such as, e.g., enhancers, poly(a) sequences, and untranslated regions (UTRs; e.g., 5′ UTR and 3′ UTR).

The composition containing the AAV vectors may be administered to the patient, for example, by local administration to the inner ear (e.g., injection into the perilymph or through the round window membrane), to treat sensorineural hearing loss.

Following administration of the composition to a patient, a practitioner of skill in the art can monitor the expression of the therapeutic protein encoded by the transgene, and the patient's improvement in response to the therapy, by a variety of methods. For example, a physician can monitor the patient's hearing by performing standard tests, such as audiometry, ABR, electrocochleography (ECOG), and otoacoustic emissions following administration of the composition. A finding that the patient exhibits improved hearing in one or more of the tests following administration of the composition compared to hearing test results prior to administration of the composition indicates that the patient is responding favorably to the treatment. Subsequent doses can be determined and administered as needed.

### OTHER EMBODIMENTS

Various modifications and variations of the described disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure that are obvious to those skilled in the art are intended to be within the scope of the disclosure. Other embodiments are in the claims.

1. A two-vector system comprising:
a) a first nucleic acid vector comprising an oncomodulin (OCM) promoter having at least 85% sequence identity to any one of SEQ ID NOs: 1-3 operably linked to a first polynucleotide encoding an N-terminal portion of a stereocilin protein; and
b) a second nucleic acid vector comprising a second polynucleotide encoding a C-terminal portion of a stereocilin protein.

2. The two-vector system of claim 1, wherein the first polynucleotide partially overlaps with the second polynucleotide.

3. The two-vector system of claim 1 or 2, wherein the first polynucleotide and the second polynucleotide have a region of overlap having a length of at least 200 bases (b).

4. The two-vector system of any one of claims 1-3, wherein the first nucleic acid vector comprises a polynucleotide comprising the sequence of nucleotides 225 to 4574 of SEQ ID NO: 43.

5. The two-vector system of any one of claims 1-4, wherein the second nucleic acid vector comprises a polynucleotide comprising the sequence of nucleotides 211 to 4219 of SEQ ID NO: 44.

6. The two-vector system of any one of claims 1-5, wherein when introduced into a mammalian cell, the first and second nucleic acid vectors undergo homologous recombination to form a recombined polynucleotide that encodes a full-length stereocilin protein.

7. The two-vector system of claim 1, wherein the first nucleic acid vector comprises a splice donor signal sequence positioned at the 3′ end of the first polynucleotide and the second nucleic acid vector comprises a splice acceptor signal sequence positioned 5′ of the second polynucleotide.

8. The two-vector system of claim 1, wherein the first nucleic acid vector comprises a splice donor signal sequence positioned at the 3′ end of the first polynucleotide and a first recombinogenic region positioned 3′ of the splice donor signal sequence and the second nucleic acid vector comprises a second recombinogenic region, a splice acceptor signal sequence 3′ of the recombinogenic region, and the second polynucleotide 3′ of the splice acceptor signal sequence.

9. The two-vector system of any one of claims 1, 7, and 8, wherein the first and second polynucleotides do not overlap.

10. The two-vector system of claim 8 or 9, wherein the first nucleic acid vector comprises a polynucleotide comprising the sequence of nucleotides 225 to 4454 of SEQ ID NO: 45 and the second nucleic acid vector comprises a polynucleotide comprising the sequence of nucleotides 257 to 3597 of SEQ ID NO: 46.

11. The two-vector system of any one of claims 8-10, wherein the first nucleic acid vector further comprises a degradation signal sequence positioned 3′ of the recombinogenic region; and wherein the second nucleic acid vector further comprises a degradation signal sequence positioned between the recombinogenic region and the splice acceptor signal sequence.

12. The two-vector system of claim 1, wherein the second nucleic acid vector further comprises an OCM promoter having at least 85% sequence identity to the nucleic acid sequence of any one of SEQ ID NOs: 1-3 operably linked to the second polynucleotide, wherein the promoter is positioned 5′ of the second polynucleotide.

13. The two-vector system of claim 1 or 12, wherein the first nucleic acid vector further comprises a polynucleotide encoding an N-terminal intein (N-intein) positioned 3′ of the first polynucleotide.

14. The two-vector system of claim 12 or 13, wherein the second nucleic acid vector further comprises a polynucleotide encoding a C-terminal intein (C-intein) positioned between the OCM promoter and the second polynucleotide.

15. The two-vector system of any one of claims 12-14, wherein the N-intein and C-intein are components of a split intein trans-splicing system.

16. The two-vector system of claim 15, wherein the split intein trans-splicing system is derived from a DnaE gene of one or more bacteria.

17. The two-vector system of any one of claims 1-16, wherein the OCM promoter has at least 85% sequence identity to SEQ ID NO: 1.

18. The two-vector system of any one of claims 1-17, wherein the two-vector system directs cochlear outer hair cell (OHC)-specific expression of a full-length stereocilin protein in a mammalian OHC.

19. The two-vector system of any one of claims 1-18, wherein the stereocilin protein is a human stereocilin protein having at least 85% sequence identity to SEQ ID NO: 4, wherein the human stereocilin protein is encoded by a polynucleotide having at least 85% sequence identity to SEQ ID NO: 6.

20. The two-vector system of any one of claims 1-19, wherein the first and second vectors are adeno-associated virus (AAV) vectors, and wherein the AAV vectors have an AAV1, AAV2, AAV2quad(Y-F), AAV3, AAV4, AAV5, AAV6, AAV7, AAV8, AAV9, AAV10, rh10, rh39, rh43, rh74, Anc80, Anc80L65, DJ/8, DJ/9, 7m8, PHP.B, PHP.eb, or PHP.S capsid.

21. A pharmaceutical composition comprising the two-vector system of any one of claims 1-20 and a pharmaceutically acceptable excipient.

22. A human OHC comprising the two-vector system of any one of claims 1-20 or the pharmaceutical composition of claim 21.

23. A method of expressing a stereocilin protein in a human OHC, comprising contacting a human OHC with the two-vector system of any one of claims 1-20 or the pharmaceutical composition of claim 21.

24. The method of claim 23, wherein the cell is in a subject.

25. A method of treating a subject having or at risk of developing sensorineural hearing loss, comprising administering to an inner ear of the subject a therapeutically effective amount of the two-vector system of any one of claims 1-20 or the pharmaceutical composition of claim 21.

26. The method of claim 25, wherein the sensorineural hearing loss is genetic sensorineural hearing loss, optionally, wherein the genetic sensorineural hearing loss is autosomal recessive hearing loss.

27. A method of increasing STRC expression in a subject in need thereof, the method comprising administering to an inner ear of the subject a therapeutically effective amount of the two-vector system of any one of claims 1-20 or the pharmaceutical composition of claim 21.

28. A method of preventing or reducing OHC damage or death in a subject in need thereof, comprising administering to an inner ear of the subject an effective amount of the two-vector system of any one of claims 1-20 or the pharmaceutical composition of claim 21.

29. A method of increasing OHC survival in a subject in need thereof, comprising administering to an inner ear of the subject an effective amount of the two-vector system of any one of claims 1-20 or the pharmaceutical composition of claim 21.

30. The method of any one of claims 25-29, wherein the subject has a mutation in STRC.

31. The method of any one of claims 25-30, wherein the subject has been identified as having a mutation in STRC.

32. The method of any one of claims 25-30, wherein the method further comprises identifying the subject as having a mutation in STRC prior to administering the two-vector system or composition.

33. The method of any one of claims 25-32, wherein the method further comprises evaluating the hearing of the subject prior to or after administering the two-vector system or composition.

34. The method of any one of claims 25-33, wherein the two-vector system or composition is administered locally to the ear.

35. The method of claim 34, wherein the vectors in the two-vector system are administered concurrently or sequentially.

36. The method of any one of claims 25-35, wherein the two-vector system or pharmaceutical composition is administered in an amount sufficient to prevent or reduce hearing loss, delay the development of hearing loss, slow the progression of hearing loss, improve hearing, improve speech discrimination, improve hair cell function, prevent or reduce hair cell damage, prevent or reduce hair cell death, promote or increase hair cell survival, or increase STRC expression in a hair cell.

37. The method of any one of claims 25-36, wherein the subject is a human.

38. A kit comprising the two-vector system of any one of claims 1-20 or the pharmaceutical composition of claim 21.
